(19) United States
(12) Patent Application Publication
(10) Pub. No.: US 2017/0348375 A1
(43) Pub. Date: Dec. 7, 2017

(54) DISLODGEMENT AND RELEASE OF HSC FROM THE BONE MARROW STEM CELL NICHE USING ALPHA9 INTEGRIN ANTAGONISTS

(71) Applicant: COMMONWEALTH SCIENTIFIC AND INDUSTRIAL RESEARCH ORGANISATION, Acton, ACT (AU)

(72) Inventors: Susan Kaye NILSSON, East Bentleigh, Victoria (AU); David Norman HAYLOCK, Melbourne, Victoria (AU); Benjamin Ben Ming CAO, Glen Waverley, Victoria (AU)

(21) Appl. No.: 15/535,117

(22) PCT Filed: Dec. 12, 2014
(86) PCT No.: PCT/AU2014/001124

§ 371 Date: Jun. 12, 2017

(51) Int. Cl. A61K 38/05; A61K 35/28; C12N 5/0789; A61K 45/06; A61K 35/12

(52) U.S. Cl. CPC A61K 38/05; C12N 5/0647; A61K 45/06; A61K 35/28; A61K 2035/124; C12N 2501/585; C12N 2509/00

(57) ABSTRACT

Haematopoietic stem cell mobilization is a process whereby haematopoietic stem cells are stimulated out of the bone marrow space. Before HSC can mobilize, they must be dislodged and released from the BM stem cell niche in which they reside and are retained by adhesive interactions. Accordingly, in an aspect of the present invention there is provided a method for enhancing dislodgement of HSC and their precursors and progenitors thereof from a BM stem cell binding ligand in vivo or ex vivo, said method comprising administering in vivo or ex vivo an effective amount of an antagonist of an a 9 integrin or an active portion thereof to the BM stem cell niche. Once mobilized to the peripheral blood (PB) the HSC may be collected for transplant. Methods which enhance mobilization of the HSC can also improve treatments of haematological disorders.

### FIELD OF THE INVENTION

The present invention relates to enhancing dislodgement and release of haematopoietic stem cells (HSC) and precursors and progenitors thereof from a bone marrow (BM) stem cell niche and methods for enhancing the dislodgement and release of HSC and their precursors and progenitors thereof from the BM and the stem cell niche. The invention also relates to compositions for use in enhancing the dislodgement and release of HSC and their precursors and progenitors thereof. Cell populations of HSC and their precursors and progenitors thereof which have been dislodged and released by the methods and compositions are included as well as the use of the cell populations for treatment of a haematological disorder and transplantation of the HSC, precursors and progenitors thereof.

### BACKGROUND OF THE INVENTION

HSC regulation and retention within the BM stem cell niche is mediated through interactions between HSC surface receptors and their respective ligands expressed by surrounding cells such as osteoblasts and sinusoidal endothelial cells. Spatial distribution analysis of HSC within BM using functional assays and in vivo and ex vivo imaging indicate they preferentially localize nearest the bone/BM interface within the endosteal niche. Of note, HSC identical to the classic Lin−Sca-1+ckit+CD150+CD48− phenotype, but isolated from endosteal BM have greater homing potential and enhanced long-term, multi-lineage haematopoietic reconstitution relative to HSC isolated from the central medullary cavity. Thus, the therapeutic targeting of endosteal HSC for mobilization should provide better transplant outcomes.

The localization of haematopoiesis to the BM involves developmentally regulated adhesive interactions between primitive haematopoietic cells and the stromal-cell-mediated haematopoietic microenvironment of the BM stem cell niche. Under steady-state conditions, HSC are retained in the BM niche by adhesive interactions with stromal elements (such as VCAM-1 and osteopontin (Opn)) leading to the physiologic retention of primitive haematopoietic progenitor cells in the BM. A perturbation of the adhesive interactions can lead to the release of the HSC retained in the BM and evoke the release of stem/progenitor cells from the bone marrow niche and eventually into the circulation by mobilization. The physiologic egress or mobilization of leukocytes from bone marrow ultimately to peripheral blood, as well as the escape of a small number of stem/progenitor cells from the normal bone marrow environment to the circulation, is a poorly understood phenomena. The movement of cells from the extravascular spaces of bone marrow to circulation may require a coordinated sequence of reversible adhesion and migration steps. The repertoire of adhesion molecules expressed by stem/progenitor cells or by stromal cells in bone marrow is crucial in this process. Alterations in the adhesion and/or migration of progenitor cells triggered by diverse stimuli would likely result in their dislodgment or redistribution between bone marrow and peripheral blood.

Releasing and mobilising specific populations of HSC may allow uses in various situations including transplantation, gene therapy, treatment of disease including cancers such as leukaemias, neoplastic cancers including breast cancers, or repair of tissues and skin. However, to mobilize HSC requires rapid and selective mobilization regimes which can initially dislodge the HSC from the BM. Dislodgement and release of specific cell populations of HSC from the BM stem cell niche can provide greater long-term, multi-lineage haematopoietic reconstitution.

The transplantation of mobilized peripheral blood (PB) haematopoietic stem cells (HSC) into patients undergoing treatment for blood diseases has essentially replaced traditional bone marrow (BM) transplants. Some clinical practices for HSC mobilization are achieved with a 5-day course of recombinant granulocyte-colony stimulating factor (G-CSF), which is believed to stimulate the production of proteases that cleave CXCR4/SDF-1 interactions. However, G-CSF is ineffective in a large cohort of patients and is associated with several side effects such as bone pain, spleen enlargement and on rare occasions, splenic rupture, myocardial infarction and cerebral ischemia.

These inherent disadvantages of G-CSF have driven efforts to identify alternate mobilization strategies based on small molecules. For example, the FDA-approved CXCR4 antagonist AMD3100 (Plerixafor; Mozobil™) has been shown to rapidly mobilize HSC with limited toxicity issues. Nevertheless, clinical mobilization with AMD3100 is only effective in combination with G-CSF and the search for rapid, selective and G-CSF independent mobilization regimens remains a topic of clinical interest. Although clinically G-CSF is the most extensively used mobilization agent, its drawbacks further include potentially toxic side effects, a relatively long course of treatment (5-7 days of consecutive injections), and variable responsiveness of patients.

However, to effect mobilization, the HSC must be released from their attachment to the BM stem cell niche. The molecules that are important in niche function and retaining the HSC in the niche environment include VCAM-1, Osteopontin (Opn) and Tenasin-C.

Integrins such as α₄β₁ have been implicated in the mobilization of HSC. Specifically both α₄β₁ (VLA-4) and α₉β₁ integrins expressed by HSC have been implicated in stem cell quiescence and niche retention through binding to VCAM-1 and osteopontin (Opn) within the endosteal region. While the role of α₉β₁ integrin in HSC mobilization is unknown, the down-regulation of Opn using non-steroidal anti-inflammatory drugs (NSAID) as well as selective inhibition of integrin α₄ or G-CSF has validated Opn/VCAM-1 binding to integrins as effective targets for HSC mobilization. However, various characteristics such as binding to small molecules such as integrins show that they are distinctly different molecules.

In Pepinsky et al (2002) the difference between α₄β₁ and α₉β₁ integrins is evident in their binding characteristics. Pepinsky shows that the differences in binding to small molecule N-benzene-sulfonyl)-(L)-prolyl-(L)-O-(1-pyrrolidinyl carbonyl) tyrosine (BOP) is evident with EGTA treatment. The treatment inhibited binding of the monoclonal antibody 9EG7 to α₄β₁, whereas it stimulated the binding of 9EG7 to α₉β₁. Most notable was the estimated >1000 fold difference in the affinity of the integrins for VCAM-1 which binds α₄β₁ with an apparent K_{d} of 10 nM and α₉β₁ with an apparent K_{d} of >10 μM. Differences were also seen in the binding of α₉β₁ and α₄β₁ to osteopontin.

Accordingly, it is an aim of the present invention to identify rapid and selective HSC dislodging and releasing compounds and regimes that are independent of G-CSF to enhance dislodgement and release of HSC which leads to improved mobilization of HSC. By targeting these compounds to specific HSC populations reconstitution and transplantation outcomes may be improved.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

In an aspect of the present invention there is provided a method for enhancing dislodgement of HSC and their precursors and progenitors thereof from a BM stem cell binding ligand in vivo or ex vivo, said method comprising administering in vivo or ex vivo an effective amount of an antagonist of an α₉ integrin or an active portion thereof to the BM stem cell niche in the presence or absence of G-CSF.

Preferably, the dislodgement of the HSC leads to release of the HSC from the BM stem cell binding ligand which enables the HSC to mobilize from the BM to the PB and thereby enhances mobilization of the HSC. Further stimulation of mobilization can be assisted by the use of mobilization agents that further enhance mobilization of HSC to the PB.

Preferably, the HSC are endosteal progenitor cells selected from the group including CD34⁺ cells, CD38⁺ cells, CD90⁺ cells, CD133⁺ cells, CD34⁺CD38⁻ cells, lineage-committed CD34⁻ cells, or CD34⁺CD38⁺ cells.

Preferably the antagonist of an α₉ integrin or an active portion thereof, is an α₉β₁ integrin or an active portion thereof.

In another embodiment, the method further includes administering an antagonist of α₄ integrin or an active portion thereof. Preferably the α₄ integrin is an antagonist of α₄β₁ or an active portion thereof.

In another embodiment, the antagonist cross-reacts with α₉ and α₄, and optionally cross-reacts with α₉β₁ and α₄β₁. Optionally, the antagonist is a α₉β₁/α₄β₁ antagonist or an active portion thereof.

Preferably, the antagonist is a compound of Formula (I) or a pharmaceutically acceptable salt thereof having the formula:

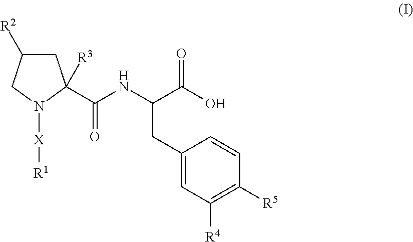

wherein
X is selected from the group consisting of a bond and —SO₂—;
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

Preferably, the compound of Formula (I) is:

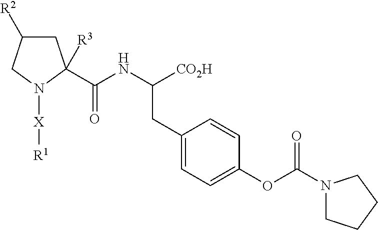

or a pharmaceutically acceptable salt thereof.

More preferably, the compound of Formula (I) is

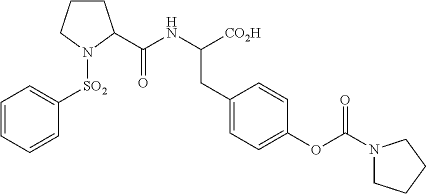 [SMILES: O=C(O)C(Cc1ccc(OC(=O)N2CCCC2)cc1)NC(=O)C1CCCN1S(=O)(=O)c1ccccc1]

or a pharmaceutically acceptable salt thereof.

Most preferably, the compound of Formula (I) is

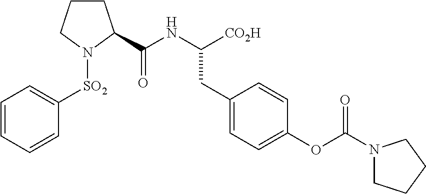 [SMILES: O=C(O)[C@H](Cc1ccc(OC(=O)N2CCCC2)cc1)NC(=O)[C@@H]1CCCN1S(=O)(=O)c1ccccc1]

or a pharmaceutically acceptable salt thereof.

In another embodiment there is provided a composition for enhancing dislodgement, release or mobilization of HSC from a BM stem cell binding ligand said composition comprising an antagonist of α₉ integrin or an active portion thereof as herein described.

In yet another aspect of the invention, there is provided a method of harvesting HSC from a subject said method comprising:
administering in the presence or absence of G-CSF an effective amount of an antagonist of α₉ integrin or an active portion thereof to a subject wherein said effective amount enhances dislodgement of HSC and their precursors and progenitors thereof from a BM stem cell binding ligand in a BM stem cell niche;
mobilizing the dislodged HSC to PB; and harvesting the HSC from the PB.

In an even further aspects of the invention methods are provided for the treatment of a haematological disorder in a subject said method comprising administering to the subject in the presence or absence of G-CSF, a therapeutically effective amount of an antagonist of α₉ integrin or an active portion thereof as herein described or a cell composition comprising HSC harvested from a subject administered with the antagonist of α₉ integrin or an active portion thereof as herein described to enhance dislodgement, release or mobilization of HSC from the BM to the PB.

In yet another preferred embodiment, the haematological disorder is a haematopoietic neoplastic disorder and the method involves chemosensitizing the HSC to alter susceptibility of the HSC, such that a chemotherapeutic agent, having become ineffective, becomes more effective.

In yet another aspect there is provided a method of transplanting HSC into a patient, said method comprising
administering an α₉ integrin antagonist to a subject to dislodge HSC from a BM stem cell binding ligand;
releasing and mobilizing the HSC from the BM to the PB;
harvesting HSC from the PB from the subject; and
transplanting the HSC to the patient.

Other aspects of the present invention will become apparent to those ordinarily skilled in the art upon review of the following description of specific embodiments of the invention.

### FIGURES

For a further understanding of the aspects and advantages of the present invention, reference should be made to the following detailed description, taken in conjunction with the accompanying drawings.

FIG. 1 shows the generation of LN18-derived cell lines. Stable LN18 cells over-expressing human integrin α₄β₁ and α₉β₁ were generated via retroviral transduction of human glioblastoma LN18 cell lines. Silencing of background α₄ expression in parental and α₉β₁ transduced LN18 cells was achieved by retroviral vector delivery of α₄ shRNA.

FIG. 2 shows antibody staining of α₄β₁ and α₉β₁ LN18 cells. Control LN18 SiA4, LN18 α₄β₁, and LN18 α₉β₁ cells were stained with mouse isotype control, mouse-anti-human α₄ antibody or mouse-anti-human α₉β₁ antibody and then secondary labelled with Alexa Fluor 594 conjugated goat-anti-mouse IgG1. Cells counterstained with DAPI (blue).

FIG. 3 shows saturation binding experiment of compound 22 and R-BC154 to control (no integrins; cross-dotted line), α₄β₁ (circle-dashed line) and α₉β₁ (square-solid line) LN18 cells with (a) compound 22 in the presence of 1 mM Ca²⁺/Mg²⁺ (open symbol) and (b) R-BC154 in the presence of either 1 mM Ca²⁺/Mg²⁺ (open symbol) or 1 mM Mn²⁺ (closed symbol). Data shown are expressed as mean fluorescence intensity (MFI)±SEM (n=3). (c) Fluorescence microscopy images of over-expressing and control LN18 cells stained with 50 nM R-BC154 (red) under Ca²⁺/Mg²⁺ and Mn²⁺ conditions. Cells were counterstained with DAPI (blue).

FIG. 4 shows cation dependent binding of R-BC154. LN18 α₉β₁ cells were treated with R-BC154 at the given concentrations in TBS buffer only (black bars), 1 mM Ca²⁺/Mg²⁺ (red bars) or 1 mM Mn²⁺ (blue bars). Data obtained is from a single experiment and is expressed as % max fluorescence.

FIG. 5 shows kinetics measurements of R-BC154 binding to LN18 cells. Association rates for binding of R-BC154 to (a) α₄β₁ (circle-dashed line) and (b) α₉β₁ (square-solid line) integrins were determined in the presence of 1 mM Ca²⁺/Mg²⁺ (open symbol) and 1 mM Mn²⁺ (closed symbol) in TBS buffer by treatment of cells with 50 nM R-BC154 for 0, 0.5, 1, 2, 3, 5, 10, 15 and 20 minutes at 37° C. (c) Dissociation rate measurements for binding of R-BC154 to α₄β₁ (circle-dashed line) and α₉β₁ (square-solid line) integrins were determined in the presence of 1 mM Ca²⁺/Mg²⁺ (open symbol) and 1 mM Mn²⁺ (closed symbol) in TBS buffer at 0, 2.5, 5, 15, 30, 45 and 60 minutes. Data shown are expressed as % mean of maximum fluorescence±SEM (n=3) and plotted as a function of time. On-rate data were fitted to a two-phase association function for all curves (R²>0.997). Off-rate data were fitted to a one-phase exponential decay function for all curves except α₄β₁ (Ca²⁺/Mg²⁺), which was fitted to a two-phase exponential decay function (R²>0.999).

FIG. 6 shows flow cytometric histogram plots of (a) bone marrow haematopoietic progenitor cells (LSK) and (b) HSC (LSKSLAM) isolated from untreated (grey lines) and R-BC154 (10 mg kg-1) injected (red lines) C57Bl/6 mice. Data is representative of 3 biological samples. Fluorescent microscopy images (inset) of FACS sorted progenitor cells (Lineage−Sca-1+c-Kit+) isolated from (c) untreated and (d) R-BC154 injected mice. Sca-1+ (blue), c-Kit+ (green), R-BC154+ (red).

FIG. 7 shows R-BC154 preferentially binds murine and human haematopoietic progenitor cells in vitro. (a) Chemical structure of R-BC154 (1). (b) Representative flow cytometry histogram plot of R-BC154 binding to control SiA₄ (α₄ knockdown; black), α₄β₁ (red) and α₉β₁ (blue) transduced LN18 cell lines in the presence of 1 mM Ca²⁺/Mg²⁺. (c) Schematic of a femur depicting endosteal (red) and central (blue) BM and a representative flow cytometry plot of BM Lin⁻Sca⁺kit⁺ (LSK) and LSKCD150⁺CD48⁻ (LSKSLAM). (d) Histogram plot of LSK and LSKSLAM cells stained with R-BC154 (10 nM) in the presence of 10 mM EDTA (deactivating; black) and 1 mM Ca²⁺/Mg²⁺ (activating; green). Unstained LSK and LSKSLAM cells are depicted in grey. (e) R-BC154 binding to LSK and LSKSLAM cells harvested from central and endosteal BM stained in the presence of 10 mM EDTA (deactivating; black) and 1 mM Ca²⁺/Mg²⁺ (activating; green). Unstained cells are depicted in grey. Data is expressed as % max mean fluorescence intensity (MFI)±SEM (n=3) and is representative of at least 3 separate experiments. (f) R-BC154 binding to central (blue) and endosteal (red) LSK cells in the absence of cations. Dotted shaded curves represent unstained LSK cells. (g) Comparative R-BC154 binding to lymphoid (B220⁺ and CD3⁺) and myeloid (Gr1Mac1⁺), LSK and LSKSLAM cells from central (blue bar) and endosteal (red bar) BM in the presence of 1 mM Ca²⁺/Mg²⁺ binding. Data is representative of 2 separate experiments. One-way ANOVA p<0.0001 (h) R-BC154 binding to central and endosteal LSK and LSKSLAM cells from wt (black bar) and α₄^{-/-}/α₉^{-/-} conditional KO mice (white bar). (i) Dose response binding of R-BC154 to human mononuclear cells (MNC) in the presence of 1 mM Ca²⁺/Mg²⁺ (green; activated) and in the absence of cations (black; non-activated). (j) Representative flow cytometry plot of human MNC expressing CD34⁺ and CD38⁻. Gated populations represent lineage committed cells (P1=CD34⁻), haematopoietic progenitor cells (P2=CD34⁺CD38⁺) and enriched stem and progenitor cells (P3=CD34⁺CD38⁻). (k) R-BC154 binding to CD34⁻, CD34⁺CD38⁻ and CD34⁺CD38⁻ cells in the presence of 1 mM Ca²⁺/Mg²⁺ (green; activating) and absence of cations (black; non-activating). Unstained cells depicted in grey. Data is from 3 individual cord blood donors and is expressed as normalized MFI (mean±SEM). *p<0.05, **p<0.01, ***p<0.005 and ****p<0.001.

FIG. 8 shows histogram plots of gated lymphoid (B220⁻ and CD3⁺), myeloid (Gr1Mac1⁺) and lineage⁻ populations from WBM treated with R-BC154 (10 nM) in the presence of 10 mM EDTA and 1 mM Ca²⁺/Mg²⁺. Unstained cells depicted in grey. Data is mean±SEM (n=3).

FIG. 9 shows R-BC154 targets HSC and progenitors via intrinsically activated α₄/α₉ integrins within the endosteal niche in situ. (a) Representative histogram plot of R-BC154 binding on gated LSK cells from central (blue) and endosteal (red) BM harvested from mice injected with R-BC154. R-BC154^{hi} population is depicted. LSK cells from uninjected mice shown in black. (b) in vivo time-course experiment depicting the proportion of R-BC154^{hi} cells within LSK and LSKSLAM cells isolated from endosteal (red) and central (blue) BM (n=3 per time point). p-value (2-way ANOVA) represents comparison between central and endosteal at the given time point. Data is mean±SEM. (c) % R-BC154^{hi} cells within lymphoid (B220⁺ and CD3⁺) and myeloid (Gr1⁺ and Mac1⁺) progenies isolated from endosteal (red bar) and central (blue bar) BM 5 mins after R-BC154 injection (n=3). Data is mean±SEM and is representative of 2 independent experiments. (d) in vivo R-BC154 binding is dependent on α₄ and α₉ integrin expression on LSK cells. Fluorescence microscopy images of lineage-depleted FACS sorted Sca-1⁺c-kit⁺ cells from R-BC154 injected wt and α₄^{-/-}/α₉^{-/-} conditional KO mice (left). R-BC154 (red); Sca-1-PB (blue); c-kit (green). Flow cytometric histogram plots of gated LSK cells from wt (red) and α₄^{-/-}/α₉^{-/-} conditional KO mice (grey) injected with R-BC154 (right). LSK cells from uninjected mice shown in black. Data is representative of 2 separate experiments. (e) Time course R-BC154 binding to LSK cells in PB and BM following subcutaneous administration. Data is mean±SEM (n=3). *p<0.05, **p<0.01, ***p<0.005 and ****p<0.001.

FIG. 10 shows (a) Analysis of the WBC content, (b) LSK content and (c) LSKSLAM content in peripheral blood of mice treated with R-BC154 at 15 and 30 mins post-injection. Data is mean±SEM and is not-significant (One-way ANOVA).

FIG. 11 shows Small molecule α₉β₁/α₄β₁ integrin antagonist BOP rapidly mobilizes HSC and progenitors. (a) Chemical structure of α₉β₁/α₄β₁ integrin antagonist BOP (2). (b) Competitive inhibition of R-BC154 binding to α₄β₁ (dotted line) and α₉β₁ (solid line) LN18 cells with BOP in the presence of 1 mM Ca²⁺/Mg²⁺. Calculated IC₅₀ values are depicted inset. (c) Competitive displacement of R-BC154 binding to endosteal LSK and LSKSLAM cells using BOP in the presence of 1 mM Ca²⁺/Mg²⁺. Data is mean±SEM (n=3) (d) Analysis of the WBC, (e) LSK and (f) LSKSLAM content in the peripheral blood of mice treated with BOP (10 mg/kg) over 90 mins. Data is mean±SEM (n=5 per time point).

### DETAILED DESCRIPTION OF THE INVENTION

Haematopoietic stem cell mobilization is a process whereby haematopoietic stem cells are stimulated out of the bone marrow space (e.g., the hip bones and the chest bone) into the bloodstream, so they are available for collection for future reinfusion or they naturally egress from the bone marrow to move throughout the body to lodge in organs such as the spleen to provide blood cells. This interesting natural phenomenon, that often accompanies various haematological disorders, may be adapted as a useful component of therapy, given the discovery of agents that can artificially incite mobilization of HSCs into the bloodstream where they can be collected and used for purposes such as transplantation. Compounds such as G-CSF and the FDA-approved CXCR-4 antagonist AMD 3100 have been shown to mobilize HSC. However, toxicity issues and various side effects can result from this treatment.

Before HSC can mobilize, they must be dislodged and released from the BM stem cell niche in which they reside and are retained by adhesive interactions.

Accordingly, in an aspect of the present invention there is provided a method for enhancing dislodgement of HSC and their precursors and progenitors thereof from a BM stem cell binding ligand in vivo or ex vivo, said method comprising administering in vivo or ex vivo an effective amount of an antagonist of an α₉ integrin or an active portion thereof to the BM stem cell niche.

In steady state conditions, HSC reside in the BM in specialized locations called the BM stem cell niche. Here they reside as quiescent stem cells before they are released ready to enter the PB and lodge in tissues to start differentiating. The HSC are retained in the BM stem cell niche by adhesion molecules or binding ligands such as but not limited to VCAM-1, Opn and Tenacin-C. Management of the HSC/BM stem cell niche interaction is instrumental the dislodgement and release of HSC to the BM stem cell niche and eventually to the PB.

Hence the present invention provides a means to dislodge and release the HSC from the interactions in the BM stem cell niche by disrupting the adhesive interactions and binding ligands between the HSC and the BM stem cell niche environment. The cells then become available for mobilizing to the PB or they may remain in the BM.

The BM stem cell niche includes the endosteal niche and the central medullary cavity. The endosteal stem cell niche is located at the endosteum of the bone marrow, where osteoblasts are the main regulators of HSC functions such as proliferation and quiescence. Furthermore, a significant proportion of HSC are closely associated with sinusoidal endothelial cells in the endothelial niche where they are ready to enter peripheral blood and start differentiation. The central medullary cavity is the central cavity of the bone responsible for the formation of red blood cells and white blood cells otherwise known as the bone marrow.

Applicants have found that by inhibiting at least the α₉ integrin with small molecule antagonists, HSC and their precursors and progenitors thereof can dislodge from the BM stem cell niche preferably into the endosteal niche or mobilize into the PB with long term multi-lineage engraftment potential. Surprisingly it has been found that the use of an antagonist to α₉ integrin or an active portion thereof significantly increases the dislodgement and release of at least CD34⁺ stem cells and progenitors into the blood.

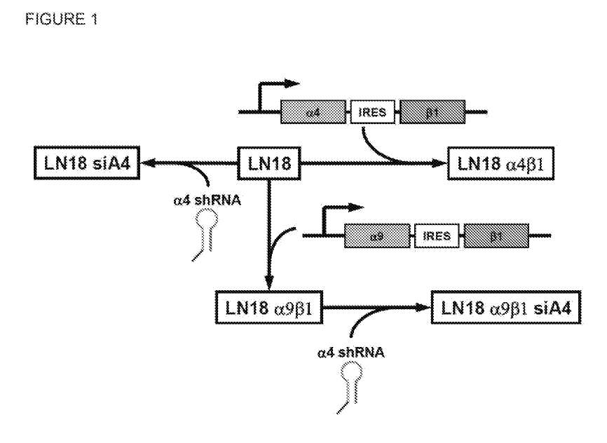

Applicants have developed a fluorescent small molecule integrin antagonist, R-BC154 (1) (FIG. 1a), based on a series of N-phenylsulfonylproline dipeptides, which bind activated human and murine α₉β₁ and α₄β₁ integrins as well as BM HSC and progenitors (FIG. 1a). Applicants postulated that this family of compounds would target potent endosteal HSC for mobilization based on the restricted interaction between α₉β₁/α₄β₁ and Opn within endosteal BM. It has now been found that R-BC154 (1) and its non-labelled derivative BOP (2) preferentially bind and mobilize mouse and human HSC and progenitors via intrinsically activated α₉β₁/α₄β₁ integrins in vivo. Thus, therapeutic targeting of endosteal HSC using α₉β₁/α₄β₁ integrin inhibitors offers a promising alternative to current mobilization strategies for stem cell transplant applications.

Integrins are non-covalently linked αβ heterodimeric trans-membrane proteins that function primarily as mediators of cell adhesion and cell signalling processes. In mammals, 18 α-chains and 8 β-chains have been identified, with at least 24 different and unique αβ combinations described to date.

The α₄β₁ integrin (very late antigen-4; VLA-4) is expressed primarily on leukocytes and are known to be receptors for vascular cell adhesion molecule-1 (VCAM-1), fibronectin and osteopontin (Opn). The α₄β₁ integrin is a key regulator of leukocyte recruitment, migration and activation and has important roles in inflammation and autoimmune disease. Accordingly, significant effort has been focused on the development of small molecule inhibitors of α₄β₁ integrin function for the treatment of asthma, multiple sclerosis and Crohn's disease, with several candidates progressing to phase I and II clinical trials.

Whilst the related β₁ integrin, α₉β₁, shares many of the structural and functional properties as α₄β₁ and also binds to several of the same ligands including VCAM-1 and Opn there are differences between the integrins α₄β₁ and α₉β₁ which make them distinct. Unlike α₄β₁ which has a restricted expression that is largely on leukocytes, the cellular expression of α₉β₁ is widespread.

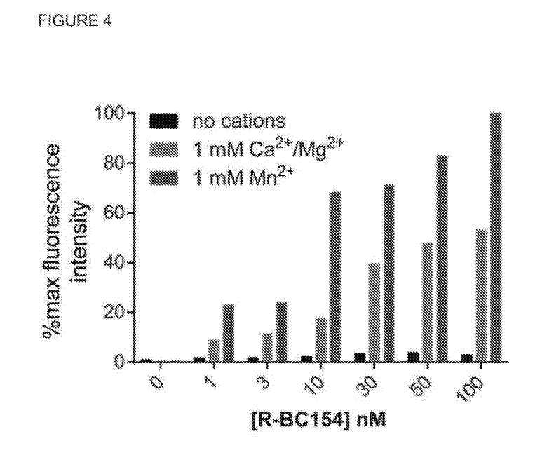

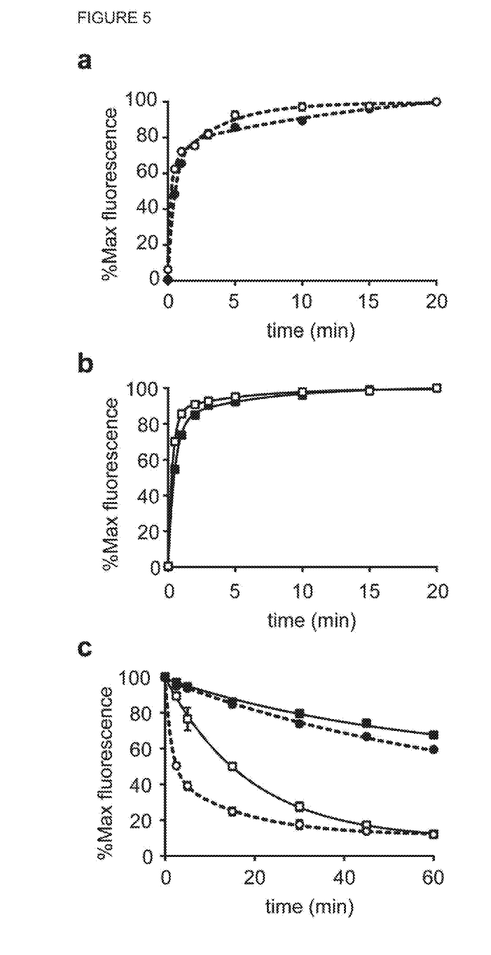

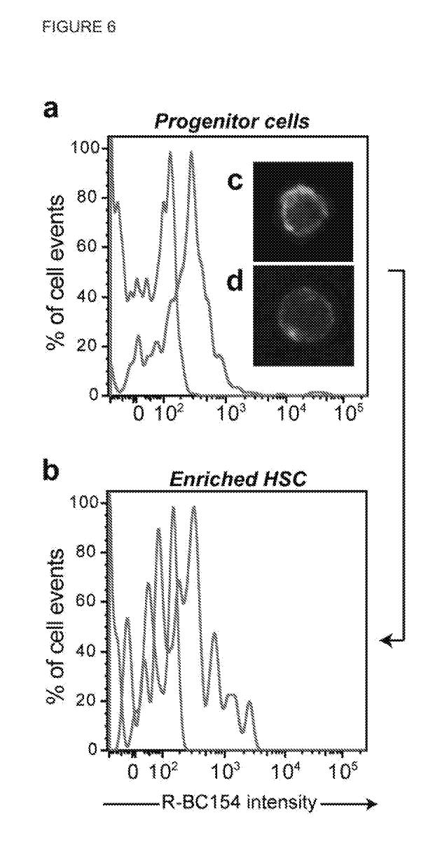

For instance binding of small molecules to α₉β₁ and α₄β₁ integrins have been shown to be different. As shown in the Examples herein, the greatest difference is in the off-rate kinetics. An α₉β₁ antagonist (R-BC154) as well as BOP are shown to have significantly reduced off-rates for α₉β₁ compared to α₄β₁. The details for R-BC154 is exemplified in Example 2 herein (FIG. 5c) and details for BOP are exemplified in Pepinsky et al (2002) (FIG. 4b).

Previously, both α₄β₁ and α₉β₁ integrins have been shown to be expressed by haemopoietic stem cells (HSC). The integrins α₄β₁ and α₉β₁ are primarily involved in the sequestration and recruitment of HSC to the bone marrow as well as the maintenance of HSC quiescence, a key characteristic for long-term repopulating stem cells.

HSC regulation by α₄β₁ and α₉β₁ integrins is mediated through interactions with VCAM-1 and Opn, which are expressed and/or secreted by bone-lining osteoblasts, endothelial cells and other cells of the bone marrow environment. However, as discussed in Pepinsky et al (2002) the difference in binding affinity for VCAM-1 and Opn are markedly different between α₄β₁ and α₉β₁. Small molecule inhibitors of α₄β₁ have been implicated as effective HSC mobilization agents. However, despite the structural and functional similarities between α₄β₁ and α₉β₁, the binding characteristics are different and hence the role of α₉β₁ integrin in this regard remains unexplored.

In one preferred embodiment of the invention, the antagonist of α₉ integrin is an antagonist of the α₉β₁ integrin. Integrin α₉ is a protein that in humans is encoded by the ITGA9 gene. This gene encodes an alpha integrin. Integrins are heterodimeric integral membrane glycoproteins composed of an alpha chain and a beta chain that mediate cell functions. The α₉ subunit forms a heterodimeric complex with a β₁ subunit to form the α₉β₁ integrin. Accordingly, it is preferred that the antagonist of α₉ integrin is an antagonist of the α₉β₁ integrin or an active portion thereof.

As used herein, an active portion of the α₉β₁ integrin or of the α₄β₁ integrin is a portion of the α₉β₁ protein or α₄β₁ protein which retains activity of the integrin. That is, the portion is a part of the α₉β₁ protein or the α₄β₁ protein which is less than the complete protein, but which can still act in the same or similar manner as the full α₉β₁ or α₄β₁ protein. Where the term “α₉ integrin” or “α₄ integrin” or “α₉β₁ integrin” or “α₄β₁ integrin” is used herein, it also includes reference to any active portions thereof.

In another embodiment of the present invention, the antagonist of α₉ integrin, preferably the α₉β₁ integrin is also an antagonist of α₄ integrin, preferably the α₄β₁ integrin. It is desired that the α₉ integrin antagonist of the present invention can inhibit the activity of both the α₉β₁ integrin and α₄β₁ integrin. Hence it is preferred that the antagonist is an α₉β₁/α₄β₁ integrin antagonist.

The antagonist of the α₉ integrin, preferably the α₉β₁ integrin may be the same or different to the antagonist of the α₄ integrin preferably the α₄β₁ integrin. If the antagonist is the same, a single antagonist may be used to inhibit the activity of both the α₉ integrin and the α₄ integrin. Separate antagonists may be used either simultaneously or sequentially to inhibit the α₉ integrin, preferably the α₉β₁ integrin and the α₄ integrin, preferably the α₄β₁ integrin.

In yet another embodiment of the invention it is preferred that the α₉ integrin, preferably the α₉β₁ integrin and the α₄ integrin preferably the α₄β₁ integrin are activated prior to the interaction of the integrin antagonist. The antagonist preferably interacts with intrinsically activated integrins. Therefore, it is desirable that the α₉ integrin is intrinsically activated. Preferably, the α₉β₁ integrin is intrinsically activated. As contemplated above, it is desirable that both the α₉β₁ integrin/α₄β₁ integrin are activated simultaneously or sequentially so that the integrin antagonist targets the HSC and progenitors via intrinsically activated α₉/α₄ integrins in the endosteal niche.

In another embodiment of the present invention, the antagonist of an α₉ integrin, preferably the antagonist of α₉β₁ integrin, more preferably a α₉β₁/α₄β₁ integrin comprises a compound of Formula (I) or a pharmaceutically acceptable salt thereof having the formula:

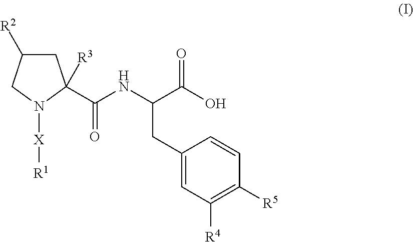

wherein
X is selected from the group consisting of a bond and —SO₂—;
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In one set of embodiments of the compound of Formula (I):
R⁴ is H;
R⁵ is —OR⁷;
   and X, R¹, R², R³ and R⁷ are as defined in Formula (I).

In such embodiments, the compound of Formula (I) may have a structure of Formula (II):

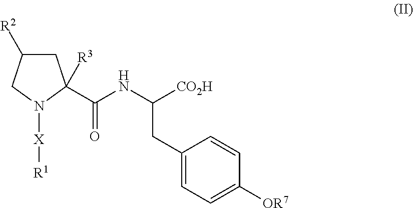

wherein:
X is selected from the group consisting of a bond and —SO₂—;
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁷ is selected from the group consisting of H, C₁-C₄ alkyl, —(CH₂)ₙ—R¹², —C(O)R¹³ and —C(O)NR¹⁴R¹⁵;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In one set of embodiments of a compound of Formula (I) or Formula (II):
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of —CN, —O(C₁-C₄ alkyl) and optionally substituted heteroaryl;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n is 1 or 2.

In some embodiments of a compound of Formula (I) or Formula (II), R⁷ is selected from C₁-C₄ alkyl.

Exemplary C₁-C₄ alkyl as described herein for groups of Formula (I) or Formula (II) may be linear or branched. In some embodiments, C₁-C₄ alkyl may be selected from the group consisting of methyl, ethyl, n-propyl, iso-propyl, n-butyl, sec-butyl, iso-butyl and tert-butyl.

In some embodiments, R⁷ may be methyl or tert-butyl, such that —OR⁷ is —OCH₃ or —OC(CH₃)₃.

In some embodiments of a compound of Formula (I) or Formula (II), R⁷ is —(CH₂)ₙ—R¹². In such embodiments, R¹² may be selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN, and n is an integer in the range of from 1 to 3.

In some embodiments of a compound of Formula (I) or Formula (II), R⁷ is —(CH₂)ₙ—R¹², where R¹² may be selected from the group consisting of —CN, —O(C₁-C₄ alkyl) and optionally substituted heteroaryl, and n is 1 or 2.

In some embodiments of a compound of Formula (I) or Formula (II), R⁷ is —(CH₂)ₙ—R¹², where:
R¹² is —OCH₃ and n is 2, or
R¹² is an optionally substituted tetrazolyl (preferably 5-tetrazolyl), and n is 1.

In some embodiments of a compound of Formula (I) or Formula (II), R⁷ is —C(O)R¹³. In such embodiments, R¹³ may be selected from the group consisting of optionally substituted cycloalkyl, optionally substituted aryl and optionally substituted heteroaryl.

In one set of embodiments, R¹³ may be an optionally substituted 5- or 6-membered cycloalkyl ring. Exemplary cycloalkyl rings may be cyclopentyl or cyclohexyl.

In one set of embodiments, R¹³ may be an optionally substituted aryl ring. An exemplary aryl ring is phenyl.

In one set of embodiments, R¹³ may be an optionally substituted heteroaryl ring. An exemplary heteroaryl ring is pyrrolyl.

In some embodiments of a compound of Formula (I) or Formula (II), R⁷ is —C(O)NR¹⁴R¹⁵.

In some embodiments of a compound of Formula (I) or Formula (II) where R⁷ is —C(O)NR¹⁴R¹⁵, R¹⁴ and R¹⁵ may each be independently selected from the group consisting of C₁-C₄ alkyl and optionally substituted aryl.

In some specific embodiments of a compound of Formula (I) or Formula (II) where R⁷ is —C(O)NR¹⁴R¹⁵, R¹⁴ and R¹⁵ are each ethyl or iso-propyl.

In one specific embodiment of a compound of Formula (I) or Formula (II) where R⁷ is —C(O)NR¹⁴R¹⁵, one of R¹⁴ and R¹⁵ is methyl and the other of R¹⁴ and R¹⁵ is phenyl.

In some embodiments of a compound of Formula (I) or Formula (II) where R⁷ is —C(O)NR¹⁴R¹⁵, R¹⁴ and R¹⁵, together with the nitrogen to which they are attached, may form an optionally substituted heterocycloalkyl ring. In one form, the optionally substituted heterocycloalkyl ring may be an optionally substituted 5- to 7-membered heterocycloalkyl ring. Particular heterocycloalkyl rings may be selected from the group consisting of pyrrolidinyl, piperidinyl, piperazinyl, and morpholinyl rings.

In specific embodiments of a compound of Formula (I) or Formula (II) R⁷ is —C(O)NR¹⁴R¹⁵, where R¹⁴ and R¹⁵, together with the nitrogen to which they are attached, form an optionally substituted pyrrolidinyl ring.

In some specific embodiments of a compound of Formula (I) or Formula (II):
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of C₁-C₄ alkyl, —CN, —O(C₁-C₄ alkyl) and 5-tetrazolyl;
R¹³ is 2-pyrrolyl;
R¹⁴ and R¹⁵ are each independently C₁-C₄ alkyl or
R¹⁴ and R¹⁵, together with the nitrogen to which they are attached, form an optionally substituted pyrrolidinyl or morpholinyl ring; and
n is 1 or 2.

In one set of embodiments of a compound of Formula (I), X is —SO₂—. In such embodiments, the compound of Formula (I) may have a structure of Formula (III):

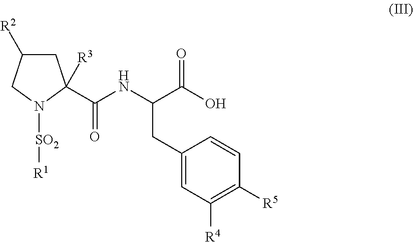

wherein:
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In some embodiments of a compound of Formula (III), R⁴ is H and R⁵ is OR⁷ to provide a compound of Formula (IIIa):

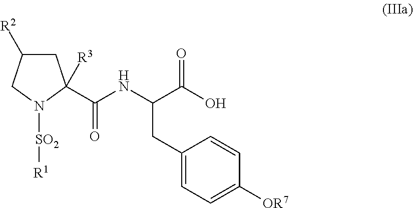

wherein
R¹, R² and R³ are as defined in Formula (III);
R⁷ is selected from the group consisting of H, C₁-C₄ alkyl, —(CH₂)ₙ—R¹², —C(O)R¹³ and —C(O)NR¹⁴R¹⁵;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In some embodiments of Formula (IIIa), R⁷ is selected from the group consisting of C₁-C₄ alkyl (preferably methyl or tert-butyl), —(CH₂)ₙ—R¹², —C(O)R¹³ and —C(O)NR¹⁴R¹⁵; wherein
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n is an integer selected from the group consisting of 1, 2 and 3.

In specific embodiments of Formula (IIIa), R⁷ is —C(O)NR¹⁴R¹⁵, where R¹⁴ and R¹⁵, together with the nitrogen to which they are attached, form an optionally substituted heterocycloalkyl ring. In one form, the optionally substituted heterocycloalkyl ring may be an optionally substituted 5- to 7-membered heterocycloalkyl ring. Particular heterocycloalkyl rings may be selected from the group consisting of pyrrolidinyl, piperidinyl, piperazinyl, and morpholinyl rings.

In a specific embodiment of Formula (I), X is —SO₂—, R⁴ is H and R⁵ is —OR⁷, where R⁷ is —C(O)NR¹⁴R¹⁵ and R¹⁴ and R¹⁵, together with the nitrogen to which they are attached, form a pyrrolidinyl ring. In such embodiments, the compound of Formula (I) may have a structure of Formula (IIIb):

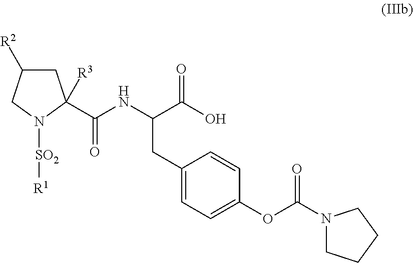

wherein R¹, R² and R³ are as defined herein.

In one set of embodiments of a compound of Formulae (I), (II), (III), (IIIa) or (IIIb) described herein, R¹ is an optionally substituted aryl. In some embodiments R¹ is an optionally substituted phenyl.

In one set of embodiments, R¹ is phenyl substituted with at least one halogen group. Halogen substituent groups may be selected from the group consisting of chloro, fluoro, bromo or iodo, preferably chloro.

In some embodiments, R¹ is phenyl substituted with a plurality of halogen groups. The halogen substituent groups may be positioned at the 3- and 5-positions of the phenyl ring.

In one embodiment, a compound of Formula (I) may have a structure of Formula (IVa) or (IVb):

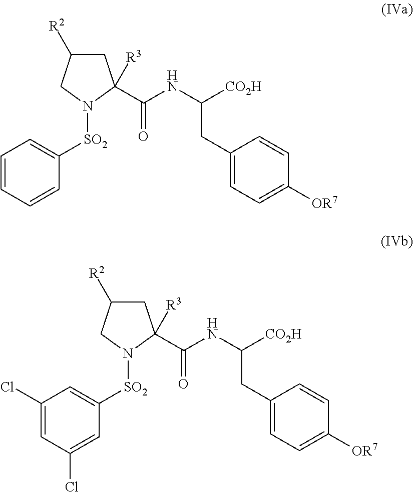

wherein in each of (IVa) and (IVb), R², R³ and R⁷ are as defined in Formula (I).

In one set of embodiments of a compound of Formula (IVa) or (IVb):
R⁷ is selected from the group consisting of H, C₁-C₄ alkyl, —(CH₂)ₙ—R¹², —C(O)R¹³ and —C(O)NR¹⁴R¹⁵;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In some embodiments of a compound of Formula (I), (II), (III), (IIIa), (IIIb), (IVa), or (IVb) as described herein, R³ is H.

In embodiments where R³ is H, the compound of Formula (I) may have a structure of Formula (V):

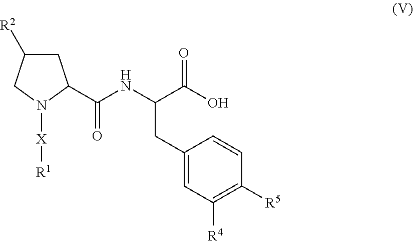

wherein:
X is selected from the group consisting of a bond and —SO₂—;
R¹ is selected from the group consisting of H, alkyl, optionally substituted aryl and optionally substituted heteroaryl;
R² is selected from the group consisting of H and a substituent group; R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In some embodiments of a compound of Formula (V), R⁴ is H and R⁵ is OR⁷ to provide a compound of Formula (Va):

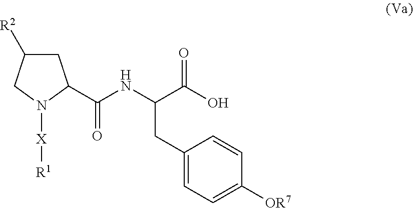

wherein
X, R¹, R² and R⁷ are as defined in Formula (V).

In some embodiments of a compound of Formula (Va), R⁷ is selected from the group consisting of C₁-C₄ alkyl (preferably methyl or tert-butyl), —(CH₂)ₙ—R¹², —C(O)R¹³ and —C(O)NR¹⁴R¹⁵; wherein R¹², R¹³, R¹⁴, R¹⁵ and n are as defined herein for Formula (V).

In specific embodiments of a compound of Formula (Va), R⁷ is —C(O)NR¹⁴R¹⁵, where R¹⁴ and R¹⁵, together with the nitrogen to which they are attached, form an optionally substituted heterocycloalkyl ring. In one form, the optionally substituted heterocycloalkyl ring may be an optionally substituted 5- to 7-membered heterocycloalkyl ring. Particular heterocycloalkyl rings may be selected from the group consisting of pyrrolidinyl, piperidinyl, piperazinyl, and morpholinyl rings.

In some embodiments of a compound of Formula (V) or (Va), X is —SO₂—.

In a specific embodiment of a compound of Formula (Va), X is —SO₂—, R³ and R⁴ are each H and R⁵ is —OR⁷, where R⁷ is —C(O)NR¹⁴R¹⁵ and R¹⁴ and R¹⁵, together with the nitrogen to which they are attached, form a pyrrolidinyl ring. In such embodiments, the compound of Formula (V) may have a structure of Formula (Vb):

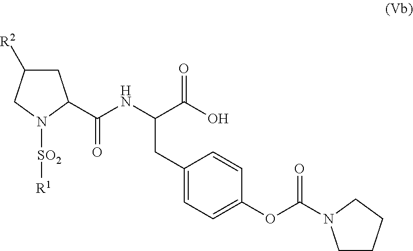

In one set of embodiments of a compound of Formula (V) or (Va), R¹ is an optionally substituted aryl, preferably an optionally substituted phenyl. The optional substituent is preferably at least one halogen group selected from the group consisting of chloro, fluoro, bromo or iodo, preferably chloro.

In one set of embodiments, R¹ is phenyl substituted with at least one halogen group. In some embodiments, R¹ is phenyl substituted with a plurality of halogen groups. The halogen substituent groups are preferably positioned at the 3- and 5-positions of the phenyl ring.

In one embodiment, a compound of Formula (V) may have a structure of Formula (VIa) or (VIb):

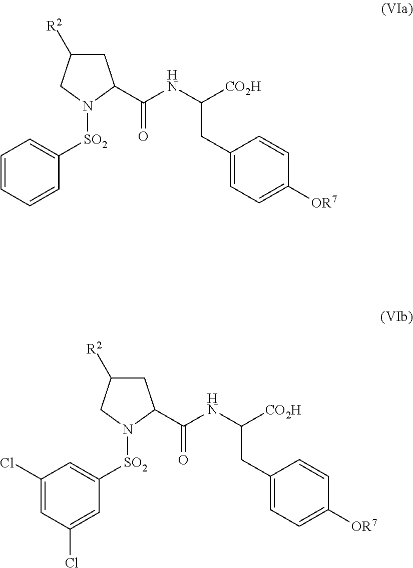

wherein in each of (VIa) and (VIb), R² and R⁷ are as defined in Formula (V).

In one set of embodiments of a compound of Formula (VIa) or (VIb):
R⁷ is selected from the group consisting of H, C₁-C₄ alkyl, —(CH₂)ₙ—R¹², —C(O)R¹³ and —C(O)NR¹⁴R¹⁵;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

In another set of embodiments of a compound of Formula (VIa) or (VIb), R⁷ is selected from the group consisting of methyl, tert-butyl, —(CH₂)ₙ—R¹² where R¹² is selected from the group consisting of —CN, —CH₃, —C(CH₃)₃ and optionally substituted heteroaryl (preferably 5-tetrazolyl), and n is 1 or 2.

In another set of embodiments of a compound of Formula (VIa) or (VIb), R⁷ is —C(O)R¹³, where R¹³ is selected from the group consisting of optionally substituted cycloalkyl (preferably cyclopentyl or cyclohexyl), optionally substituted aryl (preferably phenyl) and optionally substituted heteroaryl (preferably pyrrolyl).

In another set of embodiments of Formula (VIa) or (VIb), R⁷ is —C(O)NR¹⁴R¹⁵, where R¹⁴ and R¹⁵, together with the nitrogen to which they are attached, form an optionally substituted heterocycloalkyl ring. In one form, the optionally substituted heterocycloalkyl ring may be an optionally substituted 5- to 7-membered heterocycloalkyl ring. Particular heterocycloalkyl rings may be selected from the group consisting of pyrrolidinyl, piperidinyl, piperazinyl, and morpholinyl rings.

In a specific embodiment, a compound of Formula (I) has a structure of Formula (VII):

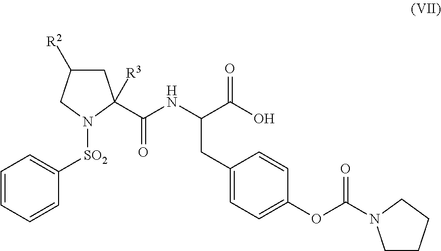

wherein R² and R³ are as defined in Formula (I).

In one set of embodiments of a compound of Formula (I), R³ is H, which provides compounds of the following formula (VIII):

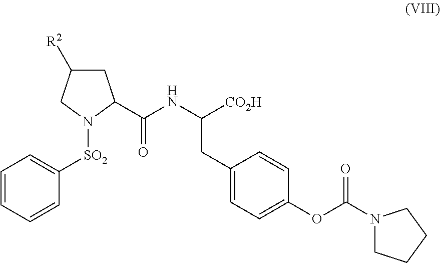

wherein R² is selected from the group consisting of H and a substituent group.

In one form of a compound of Formula (I), R² is H, which provides a compound of the following formula (IX):

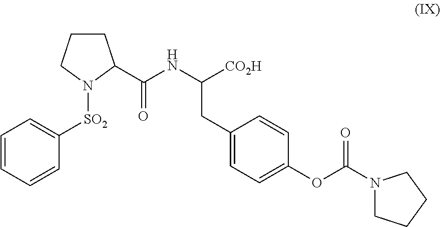 [SMILES: O=C(O)C(Cc1ccc(OC(=O)N2CCCC2)cc1)NC(=O)C1CCCN1S(=O)(=O)c1ccccc1]

or a pharmaceutically acceptable salt thereof.

In a preferred specific embodiment, the compound of Formula (I) is a compound of the following formula:

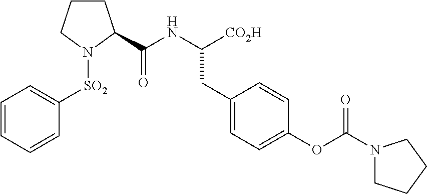 [SMILES: O=C(O)[C@H](Cc1ccc(OC(=O)N2CCCC2)cc1)NC(=O)[C@@H]1CCCN1S(=O)(=O)c1ccccc1]

or a pharmaceutically acceptable salt thereof.

As described herein, in a compound of Formulae (I), (II), (III), (IIIa), (IIIb), (IVa), (IVb), (V), (Va), (Vb), (VIa), (VIb), (VII) or (VIII), R² may in some embodiments be a substituent group.

In one set of embodiments R² is a substituent group selected from the group consisting of optionally substituted heteroaryl, optionally substituted heterocycloalkyl, optionally substituted cycloalkyl, hydroxy, amino and azido, or R² is a substituent having structure of Formula (A):

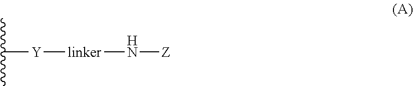

wherein
Y is optionally substituted heteroaryl or optionally substituted heteroaryl-C(O)NH—;
linker is selected from the group consisting of —(CH₂)ₚ— and —(CH₂CH₂O)—, or any combination thereof;
p at each occurrence is an integer in the range of from 1 to 4; and
Z is a fluorophore (preferably a rhodamine group).

In some embodiments of a compound of Formulae (I), (II), (III), (IIIa), (IIIb), (IVa), (IVb), (V), (Va), (Vb), (VIa), (VIb), (VII) or (VIII), R² is an optionally substituted heteroaryl. Suitable optionally substituted heteroaryl may comprise from 5 to 10 ring atoms and at least one heteroatom selected from the group consisting of O, N, and S. The optionally substituted heteroaryl may be monocyclic or bicyclic.

In some embodiments, R² may be a heteroaryl selected from the group consisting of pyrazole, imidazole, 1,2,3-triazole, 1,2,4-triazole, tetrazole, indazole, 4,5,6,7-tetrahydroindazole and benzimidazole,

In some embodiments of a compound of Formulae (I), (II), (III), (IIIa), (IIIb), (IVa), (IVb), (V), (Va), (Vb), (VIa), (VIb), (VII) or (VIII), R² is an optionally substituted heterocycloalkyl. Suitable optionally substituted heterocycloalkyl may comprise from 3 to 10 ring atoms, preferably from 4 to 8 ring atoms, and at least one heteroatom selected from the group consisting of O, N, and S. The optionally substituted heterocycloalkyl may be monocyclic or bicyclic.

In some embodiments, R² may be an optionally substituted heterocycloalkyl selected from the group consisting of optionally substituted azetidine, pyrrolidine, piperidine, azepane, morpholine and thiomorpholine.

In some embodiments, R² may be optionally substituted piperidine. In some embodiments, the piperidine may be substituted with at least one C₁-C₄ alkyl substituent group. In some embodiments, the C₁-C₄ alkyl substituent group may be methyl.

In some embodiments R² may be selected from the group consisting of 2-methylpiperidine, 3-methylpiperidine, 4-methylpiperidine, 3,5-dimethylpiperidine and 3,3-dimethylpiperidine.

When R² is a optionally substituted heteroaryl or optionally substituted heterocycloalkyl group, R² may be linked to the pyrrolidine ring of the compound of Formulae (I), (II), (III), (IIIa), (IIIb), (IVa), (IVb), (V), (Va), (Vb), (VIa), (VIb), (VII) or (VIII), via a heteroatom on the heteroaryl or heterocycloalkyl ring. For example, when R² is a heteroaryl selected from the group consisting of pyrazole, imidazole, 1,2,3-triazole, 1,2,4-triazole, tetrazole, indazole, 4,5,6,7-tetrahydroindazole and benzimidazole, or when R² is a optionally substituted heterocycloalkyl selected from the group consisting of optionally substituted azetidine, pyrrolidine, piperidine, azepane, morpholine and thiomorpholine, then R² is covalently linked to the remainder of the compound via the nitrogen (N) heteroatom of the heteroaryl or heterocycloalkyl group.

In some embodiments of a compound of Formulae (I), (II), (III), (IIIa), (IIIb), (IVa), (IVb), (V), (Va), (Vb), (VIa), (VIb), (VII) or (VIII), R² is a substituent group having structure of Formula (A):

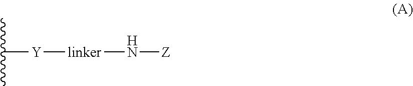

wherein
Y is optionally substituted heteroaryl; or optionally substituted heteroaryl-C(O)NH—;
linker is selected from the group consisting of —(CH₂)ₚ— and —(CH₂CH₂O)—, or any combination thereof;
p at each occurrence is an integer in the range of from 1 to 4; and
Z is a fluorophore (preferably a rhodamine group).

In some embodiments Y may be selected from the group consisting of triazole or triazole-C(O)NH—.

In some embodiments Y may be triazole or triazole-C(O)NH—, such that the structure of Formula (A) is given by Formula (A1) or (A2):

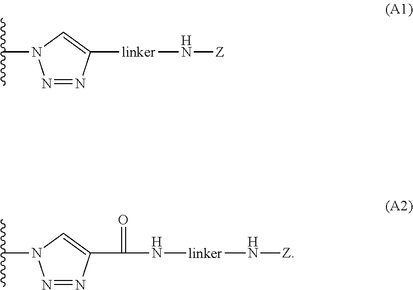

In some embodiments linker may be selected from the group consisting of —(CH₂)ₚ— and —(CH₂CH₂O)—, or any combination thereof, wherein p at each occurrence is an integer in the range of from 1 to 4.

In some embodiments linker may be given by Formula (A3) or (A4):

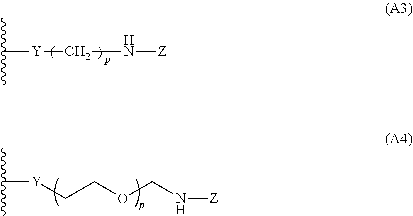

wherein p at each occurrence is an integer in the range of from 1 to 4.

In some embodiments of Formulae (A), (A1), (A2), (A3) or (A4), Z is a rhodamine fluorophore, which is selected from the following group:

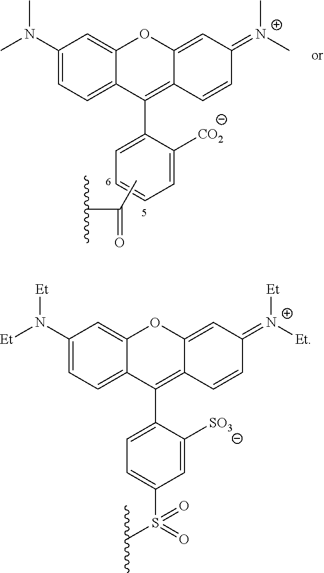

In a specific embodiment, a compound of Formula (I) has the following structure:

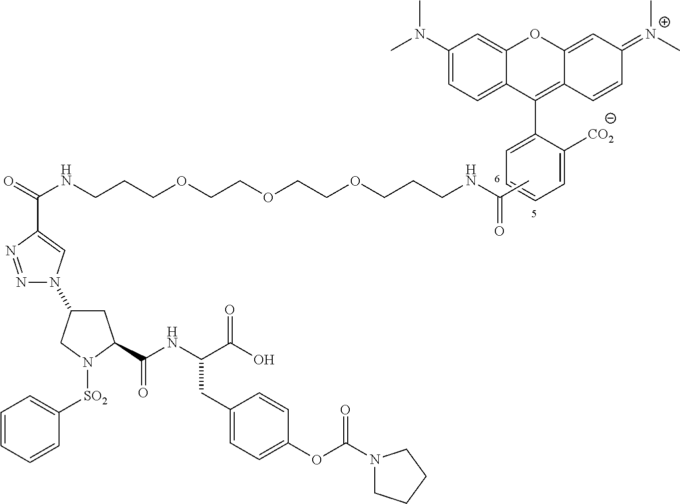

In another specific embodiment, a compound of Formula (I) has the following structure:

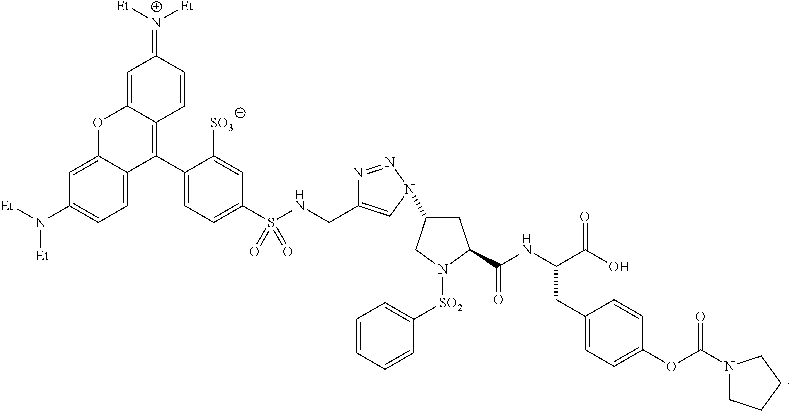

Without wishing to be limited by theory, it is believed that the pyrrolidine carbamate moiety in compounds of formulae described herein is important for ensuring a high binding affinity to an α₉ integrin, more particularly to an α₉β₁ integrin, or an active portion thereof. It is further believed that the carboxylic acid functionality is essential for antagonist activity.

In the above description a number of terms are used which are well known to a skilled addressee. Nevertheless for the purposes of clarity a number of terms are defined as follows.

As used herein, the term “unsubstituted” means that there is no substituent or that the only substituents are hydrogen.

The term “optionally substituted” as used throughout the specification denotes that the group may or may not be further substituted or fused (so as to form a condensed polycyclic system), with one or more non-hydrogen substituent groups. In certain embodiments the substituent groups are one or more groups independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, C(═O)OH, —C(═O)R^{e}, C(═O)OR^{e}, C(═O)NR^{e}R^{f}, C(═NOH)R^{e}, C(═NR^{e})NR^{f}R^{g}, NR^{e}R^{f}, NR^{e}C(═O)R^{f}, NR^{e}C(═O)OR^{f}, NR^{e}C(═O)NR^{f}R^{g}, NR^{e}C(═NR^{f})NR^{g}R^{h}, NR^{e}SO₂R^{f}, —SR^{e}, SO₂NR^{e}R^{f}, —OR^{e}, OC(═O)NR^{e}R^{f}, OC(═O)R^{e} and acyl,
wherein R^{e} and R^{f}, R^{g} and R^{h} are each independently selected from the group consisting of H, C₁-C₄alkyl, C₁-C₁₂haloalkyl, C₂-C₁₂alkenyl, C₂-C₁₂alkynyl, C₁-C₁₀ heteroalkyl, C₃-C₆cycloalkyl, C₃-C₁₂cycloalkenyl, C₅-C₆heterocycloalkyl, C₁-C₁₂heterocycloalkenyl, C₆aryl, and C₁-C₅heteroaryl, or R^{e} and R^{f}, when taken together with the atoms to which they are attached form a cyclic or heterocyclic ring system with 3 to 12 ring atoms.

In certain embodiments, optional substituents may be selected from the group consisting of halogen, alkyl, cycloalkyl, heterocycloalkyl, aryl, heteroaryl, arylalkyl, —C(O)R^{e}, —C(O)OR^{e}, —C(O)NR^{e}R^{f}, —OR^{e}, —OC(O)NR^{e}R^{f}, OC(O)R^{e} and acyl, wherein R^{e} and R^{f} are each independently selected from the group consisting of H, C₁-C₄alkyl, C₃-C₆cycloalkyl, C₅-C₆heterocycloalkyl, C₆aryl, and C₁-C₅heteroaryl, or R^{e} and R^{f}, when taken together with the atoms to which they are attached form a cyclic or heterocyclic ring system with 3 to 12 ring atoms.

“Alkyl” as a group or part of a group refers to a straight or branched aliphatic hydrocarbon group, preferably a C₁-C₁₂ alkyl, more preferably a C₁-C₁₀ alkyl, most preferably C₁-C₄ unless otherwise noted. Examples of suitable straight and branched C₁-C₄ alkyl substituents include methyl, ethyl, n-propyl, 2-propyl, n-butyl, sec-butyl and t-butyl. The group may be a terminal group or a bridging group.

“Aryl” as a group or part of a group denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group. Typically an aryl group is a C₆-C₁₈ aryl group.

A “bond” is a linkage between atoms in a compound or molecule. In one set of embodiments of a compound of Formula (I) as described herein, the bond is a single bond.

“Cycloalkyl” refers to a saturated monocyclic or fused or spiro polycyclic, carbocycle preferably containing from 3 to 9 carbons per ring, such as cyclopropyl, cyclobutyl, cyclopentyl, cyclohexyl and the like, unless otherwise specified. It includes monocyclic systems (such as cyclohexyl), bicyclic systems such as decalin, and polycyclic systems such as adamantane. A cycloalkyl group typically is a C₃-C₁₂ alkyl group. The group may be a terminal group or a bridging group.

“Halogen” represents chlorine, fluorine, bromine or iodine.

“Heteroaryl” either alone or part of a group refers to groups containing an aromatic ring (preferably a 5- or 6-membered aromatic ring) having one or more heteroatoms as ring atoms in the aromatic ring with the remainder of the ring atoms being carbon atoms. Suitable heteroatoms may be selected from the group consisting of nitrogen, oxygen and sulphur. The group may be a monocyclic or bicyclic heteroaryl group. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl, 1-, 2-, or 3-indolyl, and 2- or 3-thienyl. A heteroaryl group is typically a C₁-C₁₈ heteroaryl group. The group may be a terminal group or a bridging group.

“Heterocycloalkyl” refers to a saturated monocyclic, bicyclic, or polycyclic ring containing at least one heteroatom selected from nitrogen, sulfur, oxygen, preferably from 1 to 3 heteroatoms in at least one ring. Each ring is preferably from 3- to 10-membered, more preferably 4- to 7-membered. Examples of suitable heterocycloalkyl include pyrrolidinyl, tetrahydrofuryl, tetrahydrothiofuranyl, piperidyl, piperazyl, tetrahydropyranyl and morpholino. The group may be a terminal group or a bridging group.

It is understood that included in the family of compounds of Formula (I) are isomeric forms including diastereomers, enantiomers and tautomers, and geometrical isomers in “E” or “Z” configuration or a mixture of E and Z isomers. It is also understood that some isomeric forms such as diastereomers, enantiomers, and geometrical isomers can be separated by physical and/or chemical methods and by those skilled in the art. For those compounds where there is the possibility of geometric isomerism the applicant has drawn the isomer that the compound is thought to be although it will be appreciated that the other isomer may be the correct structural assignment.

Some of the compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Additionally, Formula (I) is intended to cover, where applicable, solvated as well as unsolvated forms of the compounds. Thus, each formula includes compounds having the indicated structure, including the hydrated as well as the non-hydrated forms.

Formula (I) is further intended to encompass pharmaceutically acceptable salts of the compounds.

The term “pharmaceutically acceptable salt” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic, carboxylic, and sulfonic classes of organic acids, examples of which are formic, acetic, propanoic, succinic, glycolic, gluconic, lactic, malic, tartaric, citric, fumaric, maleic, alkyl sulfonic, and arylsulfonic. In a similar vein base addition salts may be prepared by ways well known in the art using organic or inorganic bases. Examples of suitable organic bases include simple amines such as methylamine, ethylamine, triethylamine and the like. Examples of suitable inorganic bases include NaOH, KOH, and the like. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

In another preferred embodiment of the invention, there is provided a method for enhancing release of HSC and their precursors and progenitors thereof from a BM stem cell binding ligand in vivo or ex vivo, said method comprising administering in vivo or ex vivo an effective amount of an antagonist of an α₉ integrin or an active portion thereof to the BM stem cell niche.

Once the HSC dislodge from the BM stem cell binding ligand they are no longer anchored to the BM and available to be released from the BM and enter a cell cycle toward proliferation and differentiation. Alternatively, they can remain in the BM and enter a cell cycle in the BM.

In a further preferred embodiment, the present invention there is provided a method for enhancing mobilization of HSC and their precursors and progenitors thereof from a BM stem cell niche in vivo or ex vivo, said method comprising administering in vivo or ex vivo an effective amount of an antagonist of an α₉ integrin or an active portion thereof to the BM stem cell niche.

By virtue of the HSC becoming dislodged and released, the HSC become available to be mobilized to the PB. The dislodgement and release is essential to enable mobilization. An enhanced release of the HSC will enable more cells as a consequence to be mobilized.

In another preferred embodiment of the invention, the methods are conducted in the presence or absence of G-CSF. Preferably, the methods are conducted in the absence of G-CSF.

Although clinically G-CSF is the most extensively used mobilization agent for HSC, its drawbacks include potentially toxic side effects, a relatively long course of treatment (5-7 days of consecutive injections), and variable responsiveness of patients. Therefore, an advantage of the invention is that effective mobilization can occur in the absence of G-CSF which substantially can avoid the toxic side effects.

“Haematopoietic stem cells” as used in the present invention means multipotent stem cells that are capable of eventually differentiating into all blood cells including, erythrocytes, leukocytes, megakaryocytes, and platelets. This may involve an intermediate stage of differentiation into progenitor cells or blast cells. Hence the terms “haematopoietic stem cells”, “HSC”, “haematopoietic progenitors”, “HPC”, “progenitor cells” or “blast cells” are used interchangeably in the present invention and describe HSCs with reduced differentiation potential, but are still capable of maturing into different cells of a specific lineage, such as myeloid or lymphoid lineage. “Haematopoietic progenitors” include erythroid burst forming units, granulocyte, erythroid, macrophage, megakaryocyte colony forming units, granulocyte, erythroid, macrophage, and granulocyte macrophage colony-forming units.

The present invention relates to enhancing the dislodgment of HSC and their precursors and progenitors thereof from a BM stem cell binding ligand. Once dislodged, the cells can be released from the BM stem cell niche where they can remain or preferably be released and mobilized to the PB. These cells have haematopoietic reconstitution capacity. The present invention provides a method to enhance mobilization of HSC assisted by the dislodgement of the HSC from the BM stem cell binding ligand preferably nearest the bone/BM interface within the endosteal niche or from the central medullary cavity. More preferably, the HSC are mobilized from the bone/BM interface within the endosteal niche as it is these cells that have been shown to give greater long term, multi-lineage haematopoietic reconstitution relative to HSC isolated from the central medullary cavity.

The type of cells that are dislodged, released or mobilized may also be found in murine populations selected from the group including BM derived progenitor enriched Lin−Sca-1+ckit+(herein referred to as LSK) cells or stem cell enriched LSKCD150+CD48− cells (herein referred to as LSKSLAM). These equivalent murine populations provide an indication of the cell types that can be dislodged, released or mobilized from the BM stem cell niche by the use of an antagonist of an α₉ integrin or an active portion thereof. Preferably, the cell types are equivalent to those found in a stem cell enriched LSKCD150+CD48− cells (LSKSLAM).

Preferably, the cells that are dislodged, released or mobilized are endosteal progenitor cells and are selected from the group comprising CD34⁺, CD38⁺, CD90⁺, CD133⁺, CD34⁺CD38⁻ cells, lineage-committed CD34⁻ cells, or CD34⁺CD38⁺ cells.

The present invention may be conducted in vivo or ex vivo. That is the antagonist of α₉, preferably an antagonist of α₉β₁, more preferably an antagonist of α₉β₁/α₄β₁ can be administered to a subject in need in vivo or to an ex vivo sample to mobilize HSC from the BM.

“Subject” as used herein includes all animals, including mammals and other animals, including, but not limited to, companion animals, farm animals and zoo animals. The term “animal” can include any living multi-cellular vertebrate organisms, a category that includes, for example, a mammal, a bird, a simian, a dog, a cat, a horse, a cow, a rodent, and the like. Likewise, the term “mammal” includes both human and non-human mammals.

The present invention relates to enhancing HSC dislodgement, release or mobilization. “Enhancement,” “enhance” or “enhancing” as used herein refers to an improvement in the performance of or other physiologically beneficial increase in a particular parameter of a cell or organism. At times, enhancement of a phenomenon may be quantified as a decrease in the measurements of a specific parameter. For example, migration of stem cells may be measured as a reduction in the number of stem cells circulating in the circulatory system, but this nonetheless may represent an enhancement in the migration of these cells to areas of the body where they may perform or facilitate a beneficial physiologic result, including, but not limited to, differentiating into cells that replace or correct lost or damaged function. At the same time, enhancement may be measured as an increase of any one cell type in the peripheral blood as a result of migration of the HSC from the BM to the PB. Enhancement may refer to a 15%, 20%, 25%, 30%, 35%, 40%, 45% or greater than 50% reduction in the number of circulating stem cells or in the alternative may represent a 15%, 20%, 25%, 30%, 35%, 40%, 45% or greater than 50% increase in the number of circulating stem cells. Enhancement of stem cell migration may result in or be measured by a decrease in a population of the cells of a non-haematopoietic lineage, such as a 15%, 20%, 25%, 30%, 35%, 40%, 45%, 50%, 55%, 60%, 70%, 75% or greater decrease in the population of cells or the response of the population of cells. Put another way, an enhanced parameter may be considered as the trafficking of stem cells. In one embodiment, the enhanced parameter is the release of stem cells from a tissue of origin such as the BM. In one embodiment, an enhanced parameter is the migration of stem cells. In another embodiment, the parameter is the differentiation of stem cells.

In one embodiment, the α₉ integrin antagonist is administered intravenously, intradermally, subcutaneously, intramuscularly, transdermally, transmucosally or intraperitoneally; optionally the antagonist is administered intravenously or subcutaneously.

In yet another aspect of the invention there is provided a composition for use in enhancing dislodgement of HSC from a BM stem cell binding ligand in a BM stem cell niche in a subject said composition comprising an antagonist of α₉ integrin as herein described. More preferably, the antagonist is an α₉ integrin antagonist as herein described. Most preferably, the antagonist is an α₄β₁/α₉β₁ antagonist as herein described.

In a preferred embodiment, the composition enhances release of HSC from a BM stem cell binding ligand in a BM stem cell niche. More preferably, the composition enhances mobility or mobilization of HSC from a BM stem cell niche to the PB.

The composition may be a pharmaceutical composition further including a pharmaceutically acceptable carrier. The antagonists of α₉ integrin as described herein may be provided in the composition alone or in combination with a further antagonist of α₉ integrin, α₄ integrin, α₉β₁ integrin, α₄β₁ integrin or it may be a combined antagonist of α₉β₁/α₄β₁ integrin. The antagonists may be the same or different, but will all act as antagonists of at least the α₉ integrin.

In another aspect of the present invention there is provided a use of an antagonist of α₉ integrin as described herein in the preparation of a medicament for enhancing dislodgement of HSC and their precursors and progenitors thereof from a BM stem cell binding ligand in a patient.

The methods described herein include the manufacture and use of compositions and pharmaceutical compositions, which include antagonists of α₉ integrin as described herein as active ingredients for enhancing dislodgement of HSC and their precursors and progenitors thereof from a BM stem cell binding ligand. Preferably the release of the HSC is enhanced. More preferably, the HSC mobilization is enhanced. Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration that are known to the skilled addressee. Supplementary active compounds can also be incorporated into the compositions, e.g., growth factors such as G-CSF.

Pharmaceutical compositions are typically formulated to be compatible with the intended route of administration. Examples of routes of administration include parenteral, e.g., intravenous, intradermal, subcutaneous, transdermal (topical), transmucosal, intraperitoneal and rectal administration. Preferably, the antagonists of α₉ integrin as described herein are administered subcutaneously.

In some embodiments, the pharmaceutical compositions are formulated to target delivery of the antagonists of α₉ integrin as described herein to the bone marrow, preferably to the BM stem cell niche, and more preferably to the endosteal niche of the BM stem cell niche. For example, in some embodiments, the antagonists of α₉ integrin as described herein may be formulated in liposomes, nanosuspensions and inclusion complexes (e.g. with cyclodextrins), which can effect more targeted delivery to the BM while reducing side effects.

The pharmaceutical compositions can be included in a container, pack, or dispenser together with instructions for administration.

In yet another aspect of the invention there is provided a method of harvesting HSC from a subject said method comprising:
administering an effective amount of an antagonist of α₉ integrin or an active portion thereof as described herein to a subject wherein said effective amount enhances dislodgement of HSC and their precursors and progenitors thereof from a BM stem cell binding ligand in a BM stem cell niche;
mobilizing the dislodged HSC to PB; and
harvesting the HSC from the PB.

Preferably, the α₉ integrin antagonist is administered in the absence of G-CSF.

The use of compounds such as α₉β₁ integrin antagonists as herein described to enhance dislodgement of HSC and their precursors and progenitors thereof from a BM stem cell binding ligand in the BM stem cell niche allows for the cells to eventually mobilize to the PB for further collection. The cells may naturally mobilize and egress from the BM or they may be stimulated to mobilize by the use of other HSC mobilizing agents such as, but not limited to interleukin-17, cyclophosphamide (Cy), Docetaxel and granulocyte-colony stimulating factor (G-CSF).

In one embodiment, it is considered that the cells once harvested can be returned to the body to supplement or replenish a patient's haematopoietic progenitor cell population (homologous or autologous transplantation) or alternatively be transplanted to another patient to replenish their haematopoietic progenitor cell population (heterologous or allogeneic transplantation). This can be advantageous, in the instance following a period where an individual has undergone chemotherapy. Furthermore, there are certain genetic conditions such as thalassemias, sickle cell anemia, Dyskeratosis congenital, Shwachman-Diamond syndrome, and Diamond-Blackfan anemia wherein HSC and HPC numbers are decreased. Hence the methods of the invention in enhancing HSC dislodgement, release or mobilization may be useful and applicable.

The recipient of a bone marrow transplant may have limited bone marrow reserve such as elderly subjects or subjects previously exposed to an immune depleting treatment such as chemotherapy. They may have a decreased blood cell level or is at risk for developing a decreased blood cell level as compared to a control blood cell level. As used herein the term control blood cell level refers to an average level of blood cells in a subject prior to or in the substantial absence of an event that changes blood cell levels in the subject. An event that changes blood cell levels in a subject includes, for example, anaemia, trauma, chemotherapy, bone marrow transplant and radiation therapy. For example, the subject has anaemia or blood loss due to, for example, trauma.

Typically, an effective amount of an α₉ integrin antagonist such as an α₉β₁ integrin antagonist, more preferably a α₉β₁/α₄β₁ integrin antagonist is administered to a donor to induce dislodgement, release or preferably mobilization of HSC from the BM and release and mobilize to the PB. Once the HSC are mobilized to the PB, collection of the blood and separation of HSC can proceed using methods generally available for blood donation, such as, but not limited to those techniques employed in Blood Banks. In some embodiments, once PB or BM is obtained from a subject who has been treated using an antagonist of α₉ integrin as described herein, the HSC can be isolated therefrom, using a standard method such as apheresis or leukapheresis.

Preferably the effective amount of the α₉ integrin antagonist is in the range of 25-1000 μg/kg body weight, more preferably 50-500 μg/kg body weight, most preferably 50-250 μg/kg body weight. The effective amount may be selected from the group including 50, 60, 70, 80, 90, 100, 110, 120, 130, 140, 150, 160, 170, 180, 190, 200, 210, 220, 230, 240 or 250 μg/kg body weight.

Dislodgement, release or preferably mobilization may occur immediately, depending on the amount of α₉ integrin antagonist used. However, the HSC may be harvested in approximately 1 hours' time after administration. The actual time and amount of the α₉ integrin antagonist may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine an effective amount through routine experimentation and use of control curves.

As considered in the present invention, the term “control curve” is considered to refer to statistical and mathematically relevant curves generated through the measurement of HSC dislodgement, release or mobilization characteristics of different concentrations of α₉ integrin antagonist under identical conditions, and wherein the cells can be harvested and counted over regular time intervals. These “control curves” as considered in the present invention can be used as one method to estimate different concentrations for administering in subsequent occasions.

As considered in the present invention, the terms “harvesting haematopoietic stem cells”, “harvesting haematopoietic progenitor cells”, “harvesting HSC” or “harvesting HPC” are considered to refer to the separation of cells from the PB and are considered as techniques to which the person skilled in the art would be aware. The cells are optionally collected, separated, and optionally further expanded generating even larger populations of HSC and differentiated progeny.

In another aspect of the present invention, there is provided a cell composition comprising HSC obtained from a method as described herein said method comprising administering an effective amount of an antagonist of α₉ integrin as herein described to enhance dislodgement, release or mobilization of HSC from the BM to the PB.

As a consequence of enhanced dislodgement of the HSC, it is postulated that more HSC can be released to the BM stem cell niche for subsequent mobilization to the PB. Therefore the cell compositions harvested from a subject that has been administered an effective amount of an antagonist of an α₉ integrin or an active portion thereof to the BM stem cell niche will be enriched with HSC.

Preferably the cell composition will be enriched with cells of the endosteal niche and are endosteal progenitor cells selected from the group comprising CD34⁺, CD38⁺, CD90⁺, CD133⁺, CD34⁺CD38⁻ cells, lineage-committed CD34⁻ cells, or CD34⁺CD38⁺ cells.

In yet another aspect of the present invention there is provided a method for the treatment of haematological disorders said method comprising administering a cell composition comprising HSC obtained from a method as described herein said method comprising administering an effective amount of an antagonist of α₉ integrin as described herein to enhance dislodgement, release or mobilization of HSC from the BM to the PB.

In yet another aspect of the present invention there is provided a method for the treatment of haematological disorders in a subject said method comprising administering a therapeutically effective amount of an antagonist of α₉ integrin as described herein to the subject to enhance dislodgement, release or mobilization of HSC from the BM to the PB.

In yet another preferred embodiment, the haematological disorder is a haemaopoietic neoplastic disorder and the method involves chemosensitizing the HSC to alter susceptibility of the HSC, such that a chemotherapeutic agent, having become ineffective, becomes more effective.

A long standing issue in the treatment of leukemia is the concept that malignant cells in a dormant state are likely to evade the effects of cytotoxic agents, rendering them capable of driving relapse. Whilst much effort has gone into understanding the control of cancer cell dormancy, very little has concentrated on the role of the microenvironment and in particular the bone marrow stem cell niche. Recently, data has emerged demonstrating that the extracellular matrix molecule osteopontin, known to anchor normal haematopoietic stem cells in the bone marrow, also plays a role in supporting leukaemic cell, in particular acute lymphoblastic leukaemia (ALL), dormancy by anchoring these in key regions of the bone marrow microenvironment. Furthermore, additional data shows that relapsed ALL have significantly elevated levels of the integrin α₄β₁. These data provided herein suggest that an agent that competes with the interaction of α₉β₁ and its extracellular matrix ligands will induce these cells into cell cycle, rendering them vulnerable to cytotoxic chemotherapy.

The methods described herein include in some embodiments methods for the treatment of subjects with haematological disorders who are in need of increased numbers of stem cells. In some other embodiments, the subject is scheduled to or intends to donate stem cells such as HSC e.g., for use in heterologous or autologous transplantation. Generally, the methods include administering a therapeutically effective amount of an antagonist of α₉ integrin as described herein, to a subject who is in need of, or who has been determined to be in need of, such treatment. Administration of a therapeutically effective amount of an antagonist of α₉ integrin preferably an α₉β₁ antagonist, more preferably an antagonist of a α₉β₁/α₄β₁ integrin as described herein for the treatment of such subjects will result in an increased number and/or frequency of HSC in the PB or BM.

“Treat,” “treating” and “treatment” as used herein refer to both therapeutic treatment and prophylactic or preventative measures, wherein the aim is to prevent or slow down (lessen) the targeted condition, disease or disorder (collectively “ailment”) even if the treatment is ultimately unsuccessful. Those in need of treatment may include those already with the ailment as well as those prone to have the ailment or those in whom the ailment is to be prevented.

An “effective amount” is an amount sufficient to effect a significant increase or decrease in the number and/or frequency of HSC in the PB or BM. An effective amount can be administered in one or more administrations, applications or dosages.

“Therapeutically effective amount” as used herein refers to the quantity of a specified composition, or active agent in the composition, sufficient to achieve a desired effect in a subject being treated. For example, this can be the amount effective for enhancing migration of HSC that replenish, repair, or rejuvenate tissue. In another embodiment, a “therapeutically effective amount” is an amount effective for enhancing trafficking of HSC, such as increasing release of HSC, as can be demonstrated by elevated levels of circulating stem cells in the bloodstream. In still another embodiment, the “therapeutically effective amount” is an amount effective for enhancing homing and migration of HSC from the circulatory system to various tissues or organs, as can be demonstrated be decreased level of circulating HSC in the bloodstream and/or expression of surface markers related to homing and migration. A therapeutically effective amount may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine a therapeutically effective amount through routine experimentation.

The compositions can be administered one from one or more times per day to one or more times per week; including once every other day. The skilled artisan will appreciate that certain factors may influence the dosage and timing required to effectively treat a subject, including but not limited to previous treatments, the general health and/or age of the subject, and other diseases present. Moreover, treatment of a subject with an effective amount of the compositions described herein can include a single treatment or a series of treatments.

In some embodiments, such administration will result in an increase of about 10-200-fold in the number of HSC in the PB.

Dosage, toxicity and therapeutic efficacy of the compounds can be determined by standard pharmaceutical procedures, e.g., in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the antagonists of α₉ integrin as described herein that achieves a half-maximal inhibition of symptoms) as determined in cell culture. Such information can be used to more accurately determine useful doses in humans. Levels in plasma may be measured, for example, by high performance liquid chromatography.

In some embodiments, the methods of treatment described herein include administering another HSC mobilizing agent, e.g., an agent selected from the group consisting of, but not limited to, interleukin-17, cyclophosphamide (Cy), Docetaxel and granulocyte-colony stimulating factor (G-CSF). Preferably, the α₉ integrin antagonist may be administered with G-CSF.

In some embodiments, the methods include administering the isolated stem cells to a subject, such as reintroducing the cells into the same subject or transplanting the cells into a second subject, e.g., an HLA type-matched second subject, an allograft

The present invention includes administering an α₉ integrin antagonist directly to a patient to mobilize their own HSC or using HSC from another donor treated with an α₉ integrin antagonist from which HSC have been harvested.

In some embodiments, the subject administered an antagonist of α₉ integrin as described herein is healthy. In other embodiments, the subject is suffering from a disease or physiological condition, such as immunosuppression, chronic illness, traumatic injury, degenerative disease, infection, or combinations thereof. In certain embodiments, the subject may suffer from a disease or condition of the skin, digestive system, nervous system, lymph system, cardiovascular system, endocrine system, or combinations thereof.

In specific embodiments, the subject suffers from osteoporosis, Alzheimer's disease, cardiac infarction, Parkinson's disease, traumatic brain injury, multiple sclerosis, cirrhosis of the liver, or combinations thereof.

Administration of a therapeutically effective amount of an antagonist of α₉ integrin as described herein may prevent, treat and/or lessen the severity of or otherwise provide a beneficial clinical benefit with respect to any of the aforementioned conditions, although the application of the methods and use of the an antagonist of α₉ integrin as described herein is not limited to these uses. In various embodiments, the compositions and methods find therapeutic utility in the treatment of, among other things, skeletal tissues such as bone, cartilage, tendon and ligament, as well as degenerative diseases, such as Parkinson's and diabetes. Enhancing the release, circulation, homing and/or migration of stem cells from the blood to the tissues may lead to more efficient delivery of HSC to a defective site for increased repair efficiency.

In some embodiments subjects that can usefully be treated using the HSC, PB or BM include any subjects who can be normally treated with a bone marrow or stem cell transplant, e.g., subjects who have cancers, e.g., neuroblastoma (cancer that arises in immature nerve cells and affects mostly infants and children), myelodysplasia, myelofibrosis, breast cancer, renal cell carcinoma, or multiple myeloma. For example, the cells can be transplanted into subjects who have cancers that are resistant to treatment with radiation therapy or chemotherapy, e.g., to restore stem cells that were destroyed by high doses of chemotherapy and/or radiation therapy used to treat the cancers or non-responders to G-CSF treatment to mobilize HSC.

In some embodiments, the subject has a haematopoietic neoplastic disorder. As used herein, the term “haematopoietic neoplastic disorders” includes diseases involving hyperplastic/neoplastic cells of haematopoietic origin, e.g., arising from myeloid, lymphoid or erythroid lineages, or precursor cells thereof. In some embodiments, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), chronic myelogenous leukemia (CML); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not Limited to Hodgkin's Disease and Medium/High grade (aggressive) Non-Hodgkin's lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. In general, the methods will include administering the cell compositions, or dislodging, releasing or mobilizing stem cells to restore stem cells that were destroyed by high doses of chemotherapy and/or radiation therapy, e.g., therapy used to treat the disorders. Alternatively, the HSC are dislodged, released or mobilized from the BM stem cell niche and chemosensitized whilst entering a cell cycle either in the BM or the PB. Preferably, the haematopoietic neoplastic disorder is ALL.

In some embodiments, the BM, PB or HSC are used to treat a subject who has an autoimmune disease, e.g., multiple sclerosis (MS), myasthenia gravis, autoimmune neuropathy, scleroderma, aplastic anemia, and systemic lupus erythematosus.

In some embodiments, the subject who is treated has a non-malignant disorder such as aplastic anemia, a hemoglobinopathy, including sickle cell anemia, or an immune deficiency disorder.

The present invention further provides a dosing regimen. In one embodiment, the dosing regimen is dependent on the severity and responsiveness of a disease state to be treated, with the course of treatment lasting from a single administration to repeated administration over several days and/or weeks. In another embodiment, the dosing regimen is dependent on the number of circulating CD34+ HSCs in the peripheral blood stream of a subject. In another embodiment, the dosing regimen is dependent on the number of circulating bone marrow-derived stem cells in the peripheral blood stream of a subject. For instance, the degree of mobility of the HSC from the BM may be dependent on the number of HSC already circulating in the PB.

The present invention further provides a method of enhancing the trafficking of HSC in a subject said method comprising administering a therapeutically effective amount of an antagonist of α₉ integrin as herein described to a subject. In one embodiment, the level of trafficking of HSC relates to the number of circulating CD34⁺ HSCs in the peripheral blood of a subject. In another embodiment, the level of trafficking of HSC relates to the number of circulating bone marrow-derived HSCs in the peripheral blood of a subject.

The present invention further provides a method of inducing a transient increase in the population of circulating HSC, such as endosteal progenitor cells and are selected from the group comprising CD34⁺, CD38⁺, CD90⁺, CD133⁺, CD34⁺CD38⁻ cells, lineage-committed CD34⁻ cells, or CD34⁺CD38⁺ cells following administration of an antagonist of α₉ integrin as described herein to a subject. In one embodiment, providing an antagonist of α₉ integrin as described herein to a subject will enhance release of that subject's HSC within a certain time period, such as less than 12 days, less than 6 days, less than 3 days, less than 2 days, or less than 1 day, less than 12 hours, less than 6 hours, less than about 4 hours, less than about 2 hours, or less than about 1 hour following administration.

In one embodiment, administration of an antagonist of α₉ integrin as described herein results in the release of HSC into the circulation from about 30 minutes to about 90 minutes following administration. Preferably, the release of HSC will be about 60 minutes following administration. In another embodiment, released HSC enter the circulatory system and increase the number of circulating HSC within the subject's body. In another embodiment, the percentage increase in the number of circulating HSC compared to a normal baseline may be about 25%, 30%, 40%, 50%, 60%, 70%, 80%, 90% or about 100% or greater than about 100% increase as compared to a control. In one embodiment, the control is a base line value from the same subject. In another embodiment, the control is the number of circulating stem cells or HSC in an untreated subject, or in a subject treated with a placebo or a pharmacological carrier.

In another aspect of the invention there is provided a method of transplanting HSC into a patient, said method comprising
administering an α₉ integrin antagonist to a subject to dislodge HSC from a BM stem cell binding ligand;
releasing and mobilizing the HSC from the BM to the PB;
harvesting HSC from the PB from the subject; and
transplanting the HSC to the patient.

In one embodiment, it is considered that the cells once harvested provide a cell composition that can be returned to the body to supplement or replenish a subject's haematopoietic progenitor cell population or alternatively be transplanted to another subject to replenish their haematopoietic progenitor cell population. This can be advantageous, in the instance following a period where an individual has undergone chemotherapy.

In one embodiment the method relates specifically to transplanting a subset of HSC. These cells have haematopoietic reconstitution capacity and reside in BM in the stem cell niche. The present invention provides a method to transplant the HSC from the stem cell niche preferably nearest the bone/BM interface within the endosteal niche or from the central medullary cavity. More preferably, the HSC are transplanted from the bone/BM interface within the endosteal niche as it is these cells that have been shown to give greater long term, multi-lineage haematopoietic reconstitution relative to HSC isolated from the central medullary cavity. Preferably the cells that are transplanted are found in the stem cell niche, more preferably the central or endosteal niche. The equivalent type of cells that may be transplanted may be found in murine populations selected from the group including BM derived progenitor enriched Lin−Sca-1+ckit+(herein referred to as LSK) cells or stem cell enriched LSKCD150+CD48− cells (herein referred to as LSKSLAM).

Preferably, the cells that are transplanted are endosteal progenitor cells and are selected from the group comprising CD34⁺, CD38⁺, CD90⁺, CD133⁺, CD34⁺CD38⁻ cells, lineage-committed CD34⁻ cells, or CD34⁺CD38⁺ cells.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

The present invention will now be more fully described by reference to the following non-limiting Examples.

### Examples

### Methods

(i) Flow Cytometry

Flow cytometric analysis was performed using an LSR II (BD Biosciences) as previously described in J. Grassinger, et al Blood, 2009, 114, 49-59. R-BC154 was detected at 585 nm and excited with the yellow-green laser (561 nm). For BM and PB analysis, up to 5×10⁶ cells were analysed at a rate of 10-20 k cell events/sec. For analysis of PB LSKSLAM, up to 1×10⁶ events were saved. Cell sorting was performed on a Cytopeia Influx (BD) as previously described in J. Grassinger, et al.

(ii) Cell Lines

Stable LN18 cells ((ATCC number: CRL-2610) over-expressing integrin α₄β₁ (LN18 α₄β₁) or α₉β₁ (LN18 α₉β₁)) were generated by retroviral transduction using the pMSCV-hITGA4-IRES-hITGB1 and pMSCV-hITGA9-IRES-hITGB1 vectors as previously described in J Grassinger, et al Blood, 2009, 114, 49-59 and were maintained in DMEM supplemented with 2 mM L-glutamate in 10% FBS. Transduced cells were selected by two rounds of FACS using 2.5 μg ml⁻¹ PE-Cy5-conjugated mouse-anti-human α₄ antibody (BD Bioscience) or 20 μg ml⁻¹ of mouse-anti-human α₉β₁ antibody (Millipore) in PBS-2% FBS, followed by 0.5 μg ml⁻¹ of PE-conjugated goat-anti-mouse IgG (BD Bio-science). Silencing of α₄ expression in LN18 and LN18 α₉β₁ cells was performed as described above using pSM2c-shITGA4 (Open Biosystems). α₄-silenced LN18 cells (control cell line; LN18 SiA4) and LN18 α₉β₁ (LN18 α₉β₁SiA4) were negatively selected for α₄ expression using FACS.

(iii) Immunohistochemistry

### (a) Antibody Staining.

LN18 SiA4 (control cell line), LN18 α₄β₁, and LN18 α₉β₁ cells were stained with 2.5 μg ml⁻¹ of mouse-anti-human α₄ antibody (BD Bioscience), 4 μg ml⁻¹ of mouse-anti-human α₉β₁ antibody (Millipore) or 4 μg ml⁻¹ of mouse isotype control (BD Bioscience) in PBS-2% FBS for one hour, followed by 5 μg ml⁻¹ of Alexa Fluor 594 conjugated goat-anti-mouse IgG1 for 1 h and then washed with PBS-2% FBS three times.

### (b) Antibody Cocktails.

For analysis of R-BC154 binding to murine progenitor cells (LSK; Lineage⁻Sca-1⁺c-kit⁺) and HSC (LSKSLAM; LSKCD150⁺CD48⁻), BM and PB cells were immunolabelled with a lineage cocktail (anti-Ter119, anti-B220, anti-CD3, anti-Gr-1, anti-Mac-1), anti-Sca-1, anti-c-kit, anti-CD48 and anti-CD150. For lineage analysis, cells were stained separately for T-cells using anti-CD3, B-cells using anti-B220, macrophages using anti-Mac-1 and granulocytes using anti-Gr-1. Alternatively, lineage analysis was also performed using a cocktail containing anti-CD3/B220 (PB conjugated) and anti-B220/Gr1/Mac-1 (AF647 conjugated), whereby B220⁺ cells were identified as +/+ cells, CD³⁺ cells are +/− and Gr1/Mac-1⁺ cells are −/+ populations. For analysis of human WBC from cord blood MNCs or BM and PB from humanised NSG mice, cells were immunolabelled with a lineage cocktail containing anti-huCD3/CD14/CD15 (all AF488 conjugated), anti-CD14/CD15/CD19/CD20 (all AF647 conjugated), anti-huCD45-PB, anti-muCD45-BV510 and anti-huCD34-PECy7. A full list of conjugated antibodies used is detailed in Table 1 and 2.

TABLE 1
Anti-mouse antibodies

| Antibody | Conjugate | Clone | Isotype | Supplier | Cat# |
|---|---|---|---|---|---|
| CD3e | FITC | 17A2 | rat IgG2b | Pharmingen | 555274 |
| CD3e | Biotin | 145-2C11 | hamster IgG | Pharmingen | 553060 |
| CD3e | PB | 17A2 | rat IgG2b | Biolegend | 100214 |
| CD3e | APC/Cy7 | 17A2 | rat IgG2b | Pharmingen | 100222 |
| CD3e | APC/Cy7 | 17A2 | rat IgG2b | Biolegend | 560590 |
| CD4, L3T4 | FITC | GK1.5 | rat IgG2b | Pharmingen | 553729 |
| CD4, L3T4 | PE | GK1.5 | rat IgG2b | Pharmingen | 553739 |
| CD4, L3T4 | PB | GK1.5 | rat IgG2b | Pharmingen | 100428 |
| CD4, L3T4 | Biotin | GK1.5 | rat IgG2b | Pharmingen | 553649 |
| CD8a, Ly-2 | Biotin | 53-6.7 | rat IgG2a | Pharmingen | 553029 |
| CD8a, Ly-2 | FITC | 53-6.7 | rat IgG2a | Pharmingen | 553031 |
| CD8a, Ly-2 | PE | 53-6.7 | rat IgG2a | Pharmingen | 553033 |
| CD8a, Ly-2 | APC | 53-6.7 | rat IgG2a | Biolegend | 100712 |
| CD11b, Mac-1 | AF647 | M1/70 | rat IgG2b | Biolegend | 101218 |
| CD11b, Mac-1 | FITC | M1/70 | rat IgG2b | Pharmingen | 553310 |
| CD11b, Mac-1 | PE | M1/70 | rat IgG2b | Pharmingen | 553311 |
| CD11b, Mac-1 | PB | M1/70 | rat IgG2b | Biolegend | 101224 |
| CD11b, Mac-1 | APCCy7 | M1/70 | rat IgG2b | Pharmingen | 557657 |
| CD45 | AF647 | 30-F11 | rat IgG2b | Biolegend | 103124 |
| CD45 | APC | 30-F11 | rat IgG2b | Pharmingen | 559864 |
| CD45 | FITC | 30-F11 | rat IgG2b | Pharmingen | 553080 |
| CD45 | Biotin | 30-F11 | rat IgG2b | Pharmingen | 553078 |
| CD45 | PB | 30-F11 | rat IgG2b | Biolegend | 103126 |
| CD45 | PE | 30-F11 | rat IgG2b | Pharmingen | 563890 |
| CD45 | PE-Cy7 | 30-F11 | rat IgG2b | Biolegend | 103114 |
| CD45 | V500 | 30-F11 | rat IgG2b | Pharmingen | 553076 |
| CD45 | BV421 | 30-F11 | rat IgG2a | BD Horizon | 563890 |
| CD45 | BV510 | 30-F11 | rat IgG2a | BD Horizon | 563891 |
| CD45 | BV650 | 30-F11 | rat IgG2a | BD Horizon | 563410 |
| CD45 | APCCy7 | 30-F11 | rat IgG2a | BD Horizon | 557659 |
| CD45R, B220 | Biotin | RA3_6B2 | rat IgG2a | Pharmingen | 553086 |
| CD45R, B220 | FITC | RA3_6B2 | rat IgG2a | Pharmingen | 553088 |
| CD45R, B220 | PB | RA3_6B2 | rat IgG2a | Biolegend | 103227 |
| CD45R, B220 | PE | RA3_6B2 | rat IgG2a | Pharmingen | 553090 |
| CD45R, B220 | AF647 | RA3_6B2 | rat IgG2a | Pharmingen | 103226 |
| CD45R, B220 | APCCy7 | RA3_6B2 | rat IgG2a | Pharmingen | 552094 |
| CD45R, B220 | V500 | RA3_6B2 | rat IgG2a | Pharmingen | 561226 |
| CD45R, B220 | BV650 | RA3_6B2 | rat IgG2a | Biolegend | 103241 |
| CD45R, B220 | BV650 | RA3_6B2 | rat IgG2a | BD Horizon | 563893 |
| CD48 | Biotin | HM48-1 | A. hamster IgG | Biolegend | 103410 |
| CD48 | FITC | HM48-1 | A. hamster IgG1 | Pharmingen | 557484 |
| CD48 | FITC | HM48-1 | A. hamster IgG1 | Biolegend | 103404 |
| CD48 | PB | HM48-1 | A. hamster IgG | Biolegend | 103418 |
| CD48 | BV421 | HM48-1 | A. hamster IgG1 | BD horizon | 562745 |
| CD48 | APC | HM48-1 | A. hamster IgG | BD Pharmingen | 562746 |
| CD117, c-kit | AF647 | 2B8 | rat IgG2b | Biolegend | 105818 |
| CD117, c-kit | APC | 2B8 | rat IgG2b | Pharmingen | 553356 |
| CD117, c-kit | Biotin | 2B8 | rat IgG2b | Pharmingen | 553353 |
| CD117, c-kit | FITC | 2B8 | rat IgG2b | Pharmingen | 553354 |
| CD117, c-kit | RE | 2B8 | rat IgG2b | Pharmingen | 553311 |
| CD150 (SLAM) | Biotin | TC15-12F12.2 | rat IgG2a | Biolegend | 115908 |
| CD150 (SLAM) | PB | TC15-12F12.2 | rat IgG2a | Biolegend | 1115924 |
| CD150 (SLAM) | PE | TC15-12F12.2 | rat IgG2a | Biolegend | 115904 |
| CD150 (SLAM) | BV421 | TC15-12F12.2 | rat IgG2a | Biolegend | 115925 |
| CD150 (SLAM) | BV650 | TC15-12F12.2 | rat IgG2a | Biolegend | 115931 |
| GR-1, Ly-6G | AF647 | RB6-8C5 | rat IgG2b | Biolegend | 108418 |
| GR-1, Ly-6G | APCCy7 | RB6-8C5 | rat IgG2b | Pharmingen | 557661 |
| GR-1, Ly-6G | Biotin | RB6-8C5 | rat IgG2b | Pharmingen | 553125 |
| GR-1, Ly-6G | FITC | RB6-8C5 | rat IgG2b | Pharmingen | 553127 |
| GR-1, Ly-6G | PE | RB6-8C5 | rat IgG2b | Pharmingen | 553128 |
| GR-1, Ly-6G | PB | RB6-8C5 | rat IgG2b | Biolegend | 108430 |
| Sca-1, Ly-6A/E | Biotin | E13-161.7 | rat IgG2a | Pharmingen | 553334 |
| Sca-1, Ly-6A/E | FITC | E13-161.7 | rat IgG2a | Pharmingen | 553335 |
| Sca-1, Ly-6A/E | PB | E13-161.7 | rat IgG2a | Biolegend | 122520 |
| Sca-1, Ly-6A/E | PE | E13-161.7 | rat IgG2a | Pharmingen | 553336 |
| Sca-1, Ly-6A/E | PE | D7 | rat IgG2a | Pharmingen | 553108 |
| Sca-1, Ly-6A/E | BV421 | D7 | rat IgG2a | Pharmingen | 108128 |
| Sca-1, Ly-6A/E | PECy7 | E13-161.7 | rat IgG2a | Biolegend | 122514 |
| Sca-1, Ly-6A/E | APC | E13-161.7 | rat IgG2a | Biolegend | 122511 |
| TER119 | APC | TER119 | rat IgG2b | Pharmingen | 557909 |
| TER119 | Biotin | TER119 | rat IgG2b | Pharmingen | 553672 |
| TER119 | FITC | TER119 | rat IgG2b | Pharmingen | 557915 |
| TER119 | PE | TER119 | rat IgG2b | Pharmingen | 553673 |
| CD45 | BV650 | 30-F11 | Rat IgG2a | BD Horizon | 563410 |

TABLE 2
Anti-human antibodies

| Antibody | Conjugate | Clone | Isotype | Supplier | Cat# |
|---|---|---|---|---|---|
| CD3 | AF647 | OKT3 | Mouse IgG2a | BioLegend | 317312 |
| CD14 | AF488 | M5E2 | Mouse IgG2a | BioLegend | 301811 |
| CD14 | AF488 | M5E2 | Mouse IgG2a | BD | 557700 |
| | | | | Biosciences | |
| CD15 | AF488 | H198 | Mouse IgM | BioLegend | 301910 |
| CD19 | AF488 | HIB19 | Mouse IgG1 | BioLegend | 557697 |
| CD19 | AF647 | HIB19 | Mouse IgG1 | BioLegend | 302220 |
| CD20 | AF488 | 2H7 | Mouse IgG2b | BioLegend | 302316 |
| CD20 | AF647 | 2H7 | Mouse IgG2b | BioLegend | 302318 |
| CD34 | FITC | 8G12 | Mouse IgG1 | BD | 348053 |
| | | | | Biosciences | |
| CD34 | PECy7 | 8G12 | Mouse IgG1 | BD | 348791 |
| | | | | Biosciences | |
| CD38 | PECy7 | HB7 | Mouse IgG1 | BD | 347687 |
| | | | | Biosciences | |
| CD45 | PB | HI30 | Mouse IgG1 | Biolegend | 304029 |
| CD45 | BV650 | HI30 | Mouse IgG1 | BD | 563717 |
| | | | | Biosciences | |
| CD45 | PE | J.33 | Mouse IgG1 | Immunotech | 2078 |

### (c) R-BC154 (25) Staining.

Cultured LN18 SiA4 (control cell line), LN18 α₄β₁, and LN18 α₉β₁ cells were treated with R-BC154 (50 nM) in TBS-2% FBS (50 mM TrisHCl, 150 mM NaCl, 2 mM glucose, 10 mM Hepes, pH 7.4) containing 1 mM CaCl₂—MgCl₂ or 1 mM MnCl₂) and incubated for 20 min at 37° C. and then washed with TBS-2% FBS three times. The stained cells were fixed with 4% paraformaldehyde in PBS for 5 min, washed with water three times and then stained with 2.5 μg ml⁻¹ of DAPI. The cells were mounted in Vectorshield, washed with water, coverslipped and stored at 4° C. overnight before images were taken under fluorescent microscope (Olympus BX51).

(iv) Saturation Binding Experiments

Cultured α₄β₁, α₉β₁ and control LN18 cells (0.5×106 cells) were treated with 100 μl of either compound 22 or 25 (R-BC154) at 0, 1, 3, 10, 30 and 100 nM in TBS-2% FBS (containing either no cations, 1 mM CaCl₂—MgCl₂ or 1 mM MnCl₂). The cells were incubated at 37° C. for 60 min, washed once with TBS-2% FBS, dry pelleted and resuspended in the relevant binding buffer for flow cytometric analysis. Mean channel fluorescence was plotted against concentration and fitted to a one-site saturation ligand binding curve using GraphPad Prism 6. The dissociation constant, K_{d} was determined from the curves.

(v) Off-Rate Kinetics Measurements

Eppendorf vials containing α₄β₁ or α₉β₁ LN18 cells (0.5×10⁶ cells) were treated with 50 nM of R-BC154 (100 μl in TBS-2% FBS containing either 1 mM CaCl₂—MgCl₂ or 1 mM MnCl₂ at 37° C. until for 30 min, washed once with the relevant binding buffer and dry pelleted. The cells were treated with 500 nM of an unlabelled competing inhibitor (100 μl, in TBS-2% FBS containing either 1 mM CaCl₂—MgCl₂ or 1 mM MnCl₂) at 37° C. for the times indicated (0, 2.5, 5, 15, 30, 45, 60 min). The cells were diluted with cold TBS-2% FBS (containing the relevant cations), pelleted by centrifugation, washed once and resuspended (˜200 μl) in binding buffer for flow cytometric analysis. Mean channel fluorescence was plotted against time and the data was fitted to either a one-phase or two-phase exponential decay function using GraphPad Prism 6. The off-rate, k_{off} was extrapolated from the curves.

(vi) On-Rate Kinetics Measurements

Eppendorf vials containing α₄β₁ or α₉β₁ LN18 cells (0.5×10⁶ cells) in 50 μl TBS-2% FBS containing either 1 mM CaCl₂—MgCl₂ or 1 mM MnCl₂ were pre-activated in a heating block for 20 min at 37° C. 100 nM R-BC154 (50 μl−final concentration=50 nM) in the relevant TBS-2% FBS (with relevant cations) was added to each tube and after 0, 0.5, 1, 2, 3, 5, 10, 15 and 20 min incubation at 37° C., the tubes were quenched by the addition of 3 ml of TBS-2% FBS (with relevant cations). The cells were washed once TBS-2% FBS (with relevant cations), pelleted by centrifugation and resuspended (200 μl) in the relevant binding buffer for flow cytometric analysis. Mean channel fluorescence was plotted against time and the data was fitted to either a one-phase or two phase association function using GraphPad Prism 6. The observed on-rate, k_{obs} was extrapolated from the curves and kₒₙ was calculated using

(k_{obs}−k_{off})/[R-BC154=50 nM].

(vii) Mice

C57Bl/6 mice were bred at Monash Animal Services (Monash University, Clayton, Australia). Mice were 6-8 weeks old and sex-matched for experiments. All experiments were approved by Monash Animal Research Platform ethics committee (MARP/2012/128).

C57Bl/6 (C57), RFP, GFP and α₄^{flox/flox}/α₉^{flox/flox} vav-cre mice were bred at Monash Animal Services (Monash University, Clayton, Australia). Red fluorescent protein (RFP) mice were provided by Professor Patrick Tam (Children's Medical Research Institute, Sydney, Australia). Conditional α₄^{flox/flox}/α₉^{flox/flox} mice were initially generated by cross breeding α₄^{flox/flox} mice (gift from Thalia Papayannopoulou, University of Washington, Department of Medicine/Hematology, Seattle, Wash.) with α₉^{flox/flox} mice (kind gift from Dean Sheppard, Department of Medicine, University of California, SF) and vav-cre mice (kind gift from Warren Alexander, WEHI Institute, Melbourne). NODSIL2Rγ^{-/-} (NSG) mice were obtained in-house (Australian Regenerative Medicine Institute). Humanised NSG mice were generated by tail vein injection of freshly sorted cord blood CD34⁺ cells (>150 k) with 2×10⁶ irradiated mononuclear support cells. After 4-5 weeks post-transplantation, NSG mice were eyebled and assessed huCD45 and muCD45, and CD34 engraftment. For transplant experimentations using C57Bl/6 mice, irradiation was performed in a split dose (5.25 Gy each) 6 hours apart, 24 hours before transplantation. A total of 2×10⁵ irradiated (15 Gy) C57 BM cells were used as carrier cells for every recipient. All experiments were approved by Monash Animal Services ethics committee.

(viii) In Vivo Bone Marrow Binding Assay

R-BC154 (25) in PBS (10 mg kg-1) was injected intravenously into C57 mice. After 5 min, bone marrow cells were isolated as previously described in D. N. Haylock et al Stem Cells, 2007, 25, 1062-1069 and J. Grassinger, et al Cytokine, 2012, 58, 218-225. Briefly, one femur, tibia and iliac crest were excised and cleaned of muscle. After removing the epi- and metaphyseal regions, bones were flushed with PBS-2% FBS to obtain whole bone marrow, which were washed with PBS-2% FBS and then immunolabelled for flow cytometry. For analysis of R-BC154 binding, the following antibody combinations were chosen to minimise emission spectra overlap. For staining progenitor cells (LSK; Lineage−Sca-1⁺c-kit⁺) and HSC (LSKSLAM; LSKCD150+CD48−), cells were labelled with a lineage cocktail (CD3, Ter-119, Gr-1, Mac-1, B220; all antibodies APC-Cy7 conjugated), anti-Sca-1-PB, anti-c-kit-AF647, anti-CD48-FITC and anti-CD150-BV650.

(ix) Haematopoietic Cell Isolation.

Populations of endosteal and central murine bone marrow cells were isolated as previously described in J. Grassinger, et al Cytokine, 2012, 58, 218-225 and D. N. Haylock et al Stem Cells, 2007, 25, 1062-1069. Briefly, one femur, tibia and iliac bone were excised and cleaned of muscle. After removing the epi- and metaphyseal regions, bones were flushed with PBS-2% FBS to obtain central bone marrow cells. Flushed long bones and epi- and metaphyseal fragments were pooled and crushed using a mortar and pestle. Bone fragments were digested with Collagenase I (3 mg/ml) and Dispase II (4 mg/ml) at 37° C. in an orbital shaker at 750 rpm. After 5 min, bone fragments were washed once with PBS and once with PBS 2% FBS to collect the endosteal bone marrow cells. Peripheral blood was collected by retro-orbital puncture and red blood cells were lysed using NH₄Cl lysis buffer for 5 min at room temperature. Isolated cell populations were washed with PBS 2% FBS and then stained for flow cytometry as described in Antibody Cocktails above.

(x) Isolation of Human CD34⁺ Cells

Mononuclear cells (MNC) were isolated from cord blood as previously described in Nilsson, S. K. et al Blood 106, 1232-1239, (2005) and Grassinger, J. et al. Blood 114, 49-59, (2009). MNCs were incubated with a lineage antibody cocktail containing mouse anti-human CD3, CD11b, CD14, CD16, CD20, CD24, and CD235a (BD) and then treated with two rounds of Dynal sheep anti-mouse IgG beads (Invitrogen, Carlsbad, Calif.) at a ratio of 2 beads per cell for 5 min and then 10 min at 4° C. with constant rotation. Enriched MNC were stained with CD34-fluorescein isothiocyanate (FITC) CD34⁺ cells purified by FACS.

(xi) In Vitro and In Vivo R-BC154 Binding.

For in vitro labelling experiments, 5×10⁶ BM cells from C57 mice, conditional α₄^{-/-}/α₉^{-/-} mice and humanised NODSCIDIL2Rγ^{-/-} mice and human cord blood MNCs were treated with R-BC154 (up to 100 nM) in PBS (0.5% BSA) containing either 1 mM CaCl₂/MgCl₂ (activating) or 10 mM EDTA (deactivating) at 40×10⁶ cells/ml for 20 mins at 4° C. Cells were washed with cold PBS (2% F BS) and then immunolabelled as described in “Antibody Cocktails” prior to flow cytometric analysis. For in vivo experiments, C57BL/6 mice, α₄^{-/-}/α₉^{-/-} vav-cre mice and humanised NODSCIDIL2Rγ^{-/-} mice received either intravenous or subcutaneous injections of R-BC154 (10 mg/kg) at 100 μl/10 gm mouse weight and analysed as described above.

R-BC154 binding analysis on sorted populations of progenitor cells (LSK cells) by fluorescence microscopy were performed wherein, BM cells harvested from untreated and R-BC154 injected mice were lineage depleted for B220, Gr-1, Mac-1 and Ter-119, stained with anti-Sca-1-PB and anti-c-kit-FITC and sorted on Sca1⁺c-kit⁺. Sorted cells were imaged using an Olympus BX51 microscope.

(xii) Competitive Inhibition Assays.

α₄β₁ and α₉β₁ LN18 cells (1-2×10⁵ cells) were treated with 50 nM of R-BC154 (80 μl in PBS-2% FBS containing 1 mM CaCl₂/MgCl₂) at 37° C. for 10 mins, washed with PBS, pelleted by centrifugation and then treated with BOP (80 μl, PBS-2% FBS containing 1 mM CaCl₂/MgCl₂) at 0, 0.01, 0.1, 0.3, 1, 10, 100 and 300 nM. Cells were incubated for 90 min at 37° C., washed with PBS, pelleted by centrifugation and resuspended in PBS (200 μl) for flow cytometric analysis. % Max mean fluorescence intensity (MFI) was plotted against the log concentration of BOP and the data fitted to a ligand binding-sigmoidal dose-response curve and IC₅₀ values obtained from graphs. For competitive displacement of R-BC154 binding to LSK and LSKSLAM cells, WBM cells isolated from mice injected with R-BC154 were treated with 500 nM BOP in PBS (containing 0.5% BSA and 1 mM CaCl₂/MgCl₂) for 45 mins at 37° C. prior to flow cytometric analysis.

(xiii) Mobilization Protocols

For mobilization experiments, all mice received subcutaneous injections at 100 μl/10 gm body weight and PB was harvested by throat bleed using EDTA coated syringes.

### (a) R-BC154 and BOP.

Mice received a single injection of freshly prepared solutions of R-BC154 and BOP in saline at the doses indicated before PB was harvested by throat bleed at the times indicated.

### (b) G-CSF.

Mice received G-CSF at 250 μg/kg twice daily (500 ug/kg/day), 6-8 hours apart for 4 consecutive days. Groups receiving G-CSF and BOP received the standard G-CSF regime as described above followed by a single injection of BOP 1 h prior to harvest. Control mice received an equal volume of saline.

(xiv) Mobilization of Humanised NODSIL2Rγ (NSG) Mice

Humanised NSG mice were generated by tail vein injection of freshly sorted cord blood CD34⁺ cells (>150 k) with 2×10⁶ irradiated mononuclear support cells. After 4-5 weeks post-transplantation, NSG mice were eyebled and assessed for huCD45 and muCD45. Under these conditions, >90% humanisation was achieved as determined by flow cytometric analysis based on % huCD45 relative to total % CD45. Humanised NSG mice were given at least 1 week to recover prior to experimentation. Mice were mobilized under the relevant conditions specified in “Mobilization protocols” and PB subsequently collected by throatbleed, lysed and immunolabelled as described in “Antibody cocktails”.

(xv) Low- and High-Proliferative Potential Colony-Forming Cell Assays

Low- and high-proliferative potential colony-forming cells (LPP-CFC and HPP-CFC, respectively) were assayed as previously described in J. Grassinger et al Cytokine, 2012, 58, 218-225 and Bartelmez, S. H. et al Experimental Hematology 17, 240-245 (1989). Briefly, mobilized PB were lysed and 4000 WBCs were plated in 35 mm Petri dishes in a double-layer nutrient agar culture system containing recombinant mouse stem cell factor and recombinant human colony-stimulating factor-1, interleukin-1α (IL-1α), and IL-3. Cultures were incubated at 37° C. in a humidified incubator at 5% O₂, 10% CO₂, 85% N₂. LPP-CFC and HPP-CFC were enumerated at 14 days of incubation as previously described in J. Grassinger, et al (2012).

(xvi) Long-Term Transplant Assays

(a) Limiting Dilution Analysis.

RFP mice were treated with BOP (n=15) and PB harvested after 1 h. PB from each donor mouse per treatment group were pooled, lysed and taken up at ⅓ of the original blood volume in PBS. Irradiated WBM filler cells (2×10⁵/mouse) were added to aliquots of lysed PB at the specified transplant volume and then topped up with PBS to allow 200 μl injection/mouse. Irradiated C57BL/6 mice were administered by tail vein injection and multi-lineage RFP engraftment assessed at 6, 12 and 20 weeks post-transplant.

(b) Competitive Primary and Secondary Transplant Assay.

RFP (n=5) and GFP (n=5) mice were treated with BOP (1 h) and G-CSF (twice daily for 4 d), respectively as described in “Mobilization protocols”. PB was then harvested and blood within RFP and GFP groups were pooled, lysed, washed and resuspended to ⅓ of the original blood volume in PBS. Equal volumes of RFP and GFP blood were mixed to allow transplantation of 500 μl of RFP and GFP blood per mouse. Irradiated WBM filler cells (2×10⁵/mouse) were added and the mixture topped up in PBS to allow 200 μl injection/mouse. Irradiated C57BL/6 recipients (n=5) were administered by tail vein injection and RFP and GFP engraftment assessed at 6, 12 and 20 weeks post-transplant. At 20 weeks takedown, WBM cells ( 1/10th of a femur) from each primary recipient (n=5) was transplanted into irradiated C57 secondary recipients (n=4/primary recipient) and assessed for multi-lineage engraftment at 6, 12 and 20 weeks post-transplant.

(xvii) Statistical Analysis

Data were analyzed using student's t-test, one-way or two-way ANOVA where appropriate for the data set. For determination of stem cell repopulation frequency, Poisson analysis using L-CALC software (Stem Cell Technologies) was performed. Log-rank (Mantel-Cox) test was used to compare survival curves. p<0.05 was considered significant.

### Example 1: Preparation of α₉β₁ Integrin Antagonists

(a) Synthesis of Antagonist Compounds

The agents of the various embodiments may be prepared using the reaction routes and synthesis schemes as described below. The preparation of particular compounds of the embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g. by appropriately protecting interfering groups, by changing to other suitable reagents known in the art, or by making routine modifications of reaction conditions. A list of suitable protecting groups in organic synthesis can be found in T. W. Greene's Protective Groups in Organic Synthesis, 3rd Edition, John Wiley & Sons, 1991. Alternatively, other reactions disclosed herein or known in the art will be recognized as having applicability for preparing other compounds of the various embodiments.

Reagents useful for synthesizing compounds may be obtained or prepared according to techniques known in the art.

The symbols, abbreviations and conventions in the processes, schemes, and examples are consistent with those used in the contemporary scientific literature. Specifically but not meant as limiting, the following abbreviations may be used in the examples and throughout the specification.
Ac (acetyl)
BOP (N-(benzenesulfonyl)-L-O-(1-pyrrolidinylcarbonyl)tyrosine)
Cbz (carboxybenzyl)
CDCl₃ (deuterated chloroform)
CHCl₃ (chloroform)
CuAAC (copper(I)-catalyzed azide alkyne cycloaddition)
DCC (N,N′-dicyclohexylcarbodiimide)
DCM (dichloromethane)
DIAD (diisopropyl azodicarboxylate)
DIPEA (diisopropyl ethyl amine)
DMF (N, N-dimethylformamide)
DMSO (dimethylsulfoxide)
EtOAc (ethyl acetate)
EtOH (ethanol)
FTIR (Fourier transform infrared)
g (grams)
h (hours)
HATU (O-(7-aza-1H-benzotriazol-1-yl)-N,N,N′,N′-tetramethyluronium hexafluorophosphate)
HBTU (O-(benzotriazol-1-yl)-N,N,N′,N′-tetramethyl uronium hexafluorophosphate)
HCl (hydrochloric acid)
HPLC (high pressure/high performance liquid chromatography)
HRMS (high resolution mass spectrometry)
Hz (Hertz)
K₂CO₃ (potassium carbonate)
L (litres)
MeOH (methanol)
mg (milligrams)
MHz (megahertz)
min (minutes)
mL (millilitres)
mM (millimolar)
mol (moles)
Ms (mesylate)
Nα₂SO₄ (sodium sulfate)
NHS (N-hydroxysuccinimide)
NMR (nuclear magnetic resonance)
PEG (polyethylene glycol)
pet. spirits (petroleum spirits)
ppm (parts per million)
psi (pounds per square inch)
S_{N}2 (substitution—nucleophilic, bimolecular)
TBTA (tris[(1-benzyl-1H-1,2,3-triazol-4-yl)methyl]amine)
TEA (triethylamine)
TFA (trifluoroacetic acid)
Tf (triflate)
THF (tetrahydrofuran)
TLC (thin layer chromatography)
UV (ultraviolet)
RM (reaction mixture)
Rₜ (retention time)
rt (room temperature)

Unless otherwise indicated, all temperatures are expressed in C (degree centigrade).

All reactions conducted at room temperature unless otherwise mentioned.

All starting materials, reagents, and solvents were obtained from commercial sources and used without further purification unless otherwise stated. N-(Benzyloxycarbonyl)-L-prolyl-L-O-(tert-butylether)tyrosine methyl ester 26 was obtained from Genscript. All anhydrous reactions were performed under a dry nitrogen atmosphere. Diethyl ether, dichloromethane, tetrahydrofuran and toluene were dried by passage through two sequential columns of activated neutral alumina on the Solvent Dispensing System built by J. C. Meyer and based on an original design by Grubbs and co-workers.

Petroleum spirits refers to the fraction boiling at 40-60° C. Thin layer chromatography (TLC) was performed on Merck pre-coated 0.25 mm silica aluminium-backed plates and visualised with UV light and/or dipping in ninhydrin solution or phosphomolybdic acid solution followed by heating. Purification of reaction products was carried out by flash chromatography using Merck Silica Gel 60 (230-400 mesh) or reverse phase C18 silica gel. Melting points were recorded on a Reichert-Jung Thermovar hot-stage microscope melting point apparatus. Optical rotations were recorded on a Perkin Elmer Model 341 polarimeter. FTIR spectra were obtained using a ThermoNicolet 6700 spectrometer using a SmartATR (attenuated total reflectance) attachment fitted with a diamond window. Proton (¹H) and carbon (¹³C) NMR spectra were recorded on a BrukerAV400 spectrometer at 400 and 100 MHz, respectively. ¹H NMR are reported in ppm using a solvent as an internal standard (CDCl₃ at 7.26 ppm). Proton-decoupled ¹³C NMR (100 MHz) are reported in ppm using a solvent as an internal standard (CDCl₃ at 77.16 ppm). High resolution mass spectrometry was acquired on either a WATERS QTOF II (CMSE, Clayton, VIC 3168) or a Finnigan hybrid LTQ-FT mass spectrometer (Thermo Electron Corp., Bio21 Institute, University of Melbourne, Parkville, VIC 3010) employing Electrospray Ionisation (ESI).

### Example 1A—Preparation of N-(Benzenesulfonyl)-L-prolyl-L-O-(1-pyrrolidinylcarbonyl)tyrosine (BOP)

Synthesis of BOP began from the dipeptide 26, as shown in the following Scheme 1:

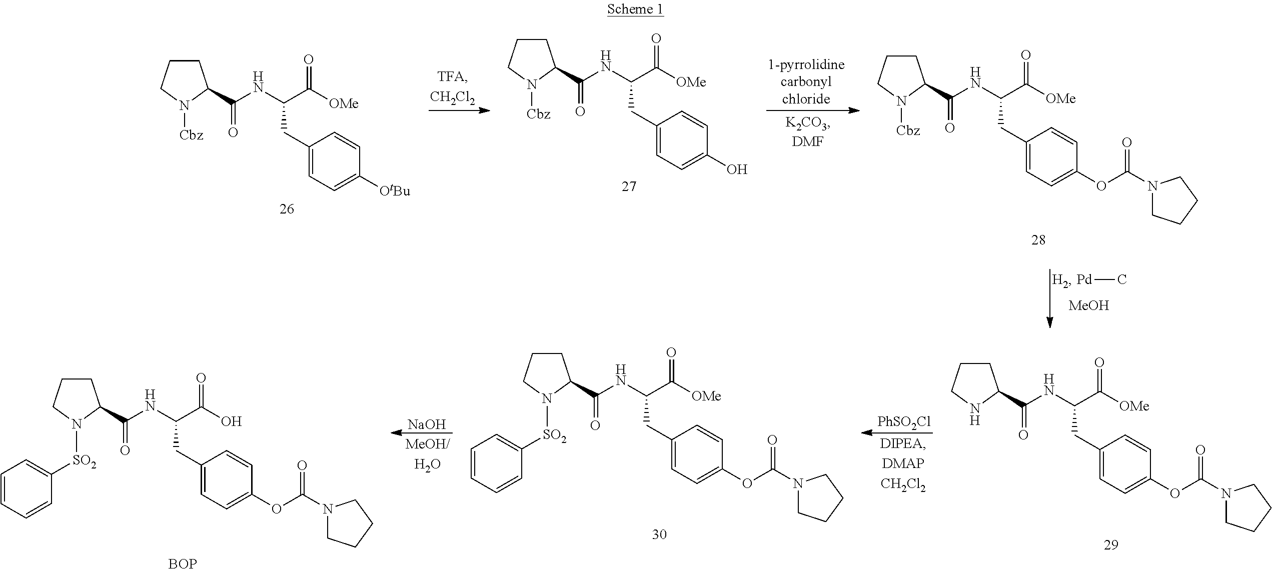

Deprotection of the tert-butyl protecting group of 26 using trifluoroacetic acid at 0° C. provided phenol 27, which was used in the next step, after aqueous work-up, without further purification. Reaction of phenol 27 with 1-pyrrolidinecarbonyl chloride proceeded smoothly in the presence of potassium carbonate to provide carbamate 28 in good yield (74%) over two steps. Hydrogenolysis of the Cbz protecting group was complete within 3 hours, and the resulting amine was obtained in excellent yield (85%) after flash chromatography. Amine 29 was then reacted with benzenesulfonyl chloride in the presence of base to give the sulfonamide 30 in excellent yield (96%) after flash chromatography. Finally, the methyl ester moiety of 30 was saponified using sodium hydroxide, followed by ion-exchange on Amberlyst resin, to provide BOP in 81% yield after flash chromatography.

By way of exemplification we provide actual reaction conditions for the formation of BOP, starting from dipeptide 26.

### Step 1: N-(Benzyloxycarbonyl)-L-prolyl-L-O-tyrosine methyl ester (27)

TFA (1.27 mL, 16.6 mmol) was added dropwise to a suspension of N-(benzyloxycarbonyl)-L-prolyl-L-O-(tert-butylether)tyrosine methyl ester 26 (0.80 g, 1.66 mmol; custom peptide synthesis from Genscript) in dry CH₂Cl₂ (10 mL) at 0° C. The mixture was slowly warmed to rt and stirred for 3 h at which point TLC (70:30 EtOAc/pet. spirits) indicated complete consumption of starting material. The mixture was diluted with EtOAc and washed with H₂O, brine, dried (MgSO₄) and concentrated under reduced pressure. The residue was concentrated with toluene (×3) to give the crude N-(benzyloxycarbonyl)-L-prolyl-L-O-tyrosine methyl ester 27 (700 mg) as a colourless oil, which was used in the next step without further purification.

### Step 2: N-(Benzyloxycarbonyl)-L-prolyl-L-O-(1-pyrrolidinylcarbonyl)tyrosine methyl ester (28)

1-Pyrrolidinecarbonyl chloride (147 μL, 1.38 mmol) was added to a mixture of the crude phenol 27 (393 mg, 0.922 mmol) and K₂CO₃ (256 mg, 1.84 mmol) in DMF (5 mL). The mixture was stirred at 50° C.overnight, diluted with EtOAc/H₂O and the organic phase separated. The organic layer was washed with 5% HCl, sat. aq. NaHCO₃, brine, dried (MgSO₄) and concentrated under reduced pressure. The residue was purified by flash chromatography (70% EtOAc/pet. spirits) to give the carbamate 28 (355 mg, 74%) as a colourless foam, which was used in the next step without further purification.

### Step 3: L-prolyl-L-O-(1-pyrrolidinylcarbonyl)tyrosine methyl ester (29)

A mixture of the Cbz protected dipeptide 28 (356 mg, 0.681 mmol) and 10% Pd/C (50% H₂O, 150 mg) in MeOH (30 mL) was purged three times with H₂. The mixture was stirred under a H₂ atmosphere for 3 h at which point TLC (10% MeOH/CH₂Cl₂) indicated complete consumption of starting material. The mixture was filtered through a layer of Celite and the filtrate concentrated under reduced pressure. The residue was purified by flash chromatography (5% to 10% MeOH/CH₂Cl₂) to give the amine 29 (224 mg, 85%) as a colourless oil. δ_{H} (400 MHz, CDCl₃) 1.64-1.78 (2H, m), 1.82-1.92 (5H, m), 2.16-2.25 (1H, m), 2.97-3.15 (4H, m), 3.39 (2H, t, J=6.5 Hz), 3.49 (2H, t, J=6.5 Hz), 3.65 (3H, s), 4.03 (1H, dd, J=5.7, 8.3 Hz) 4.72 (1H, dd, J=7.8, 13.3 Hz), 5.69 (1H, br s), 6.99 (2H, d, J=8.3 Hz), 7.13 (2H, d, J=8.3 Hz), 8.41 (1H, d, J=7.9 Hz).

### Step 4: N-(Benzenesulfonyl)-L-prolyl-L-O-(1-pyrrolidinylcarbonyl)tyrosine methyl ester (30)

DIPEA (95 μL, 0.546 mmol) was added to a stirred solution of the amine D (71 mg, 0.182 mmol), PhSO₂Cl (35 μL, 0.273 mmol) and DMAP (2.2 mg, 0.018 mmol) in CH₂Cl₂ (3 mL). The mixture was stirred for 4 h at rt, concentrated under reduced pressure and the residue purified by flash chromatography (2.5% MeOH/CH₂Cl₂) to give the product E (93 mg, 96%) as a colourless foam. δ_{H} (400 MHz, CDCl₃) 1.42-1.56 (3H, m), 1.90-2.05 (5H, m), 3.03 (1H, dd, J=7.6, 14.0 Hz), 3.10-3.16 (1H, m), 3.26 (1H, dd, J=5.6, 14.0 Hz), 3.35-3.40 (1H, m), 3.45 (2H, t, J=6.5 Hz), 3.54 (2 H, t, J=6.5 Hz), 3.77 (3H, s), 4.08 (1H, dd, J=2.0, 8.0 Hz), 4.82 (1H, dt, J=5.7, 11.6 Hz), 7.06 (2H, d, J=8.7 Hz), 7.13 (2H, d, J=8.7 Hz), 7.25 (1H, d, J=7.5 Hz; obscured by solvent peak), 7.52-7.57 (2H, m), 7.61-7.65 (1H, m), 7.83-7.85 (2H, m).

### Step 5: N-(Benzenesulfonyl)-L-prolyl-L-O-(1-pyrrolidinylcarbonyl)tyrosine (BOP)

0.1 M NaOH (3.2 mL, 0.162 mmol) was added to a solution of the ester 30 (86 mg, 0.162 mmol) in MeOH (10 mL) and the mixture stirred overnight at rt. The reaction was quenched with Amberlyst resin (H⁺ form), filtered and the filtrate concentrated under reduced pressure. The crude product was purified by flash chromatography (10% MeOH/CH₂Cl₂) to give the product BOP (68 mg, 81%) as a colourless glass. δ_{H} (400 MHz, d₄-MeOH) 1.47-1.55 (1H, m), 1.59-1.72 (2H, m), 1.77-1.85 (1H, m), 1.93-2.00 (4H, m), 3.11 (1H, dd, J=7.8, 13.7 Hz), 3.18-3.24 (1H, m), 3.27 (1H, dd, J=5.0, 13.7 Hz), 3.35-3.44 (3H, m), 3.56 (2H, d, J=6.5 Hz), 4.14 (1H, dd, J=4.0, 8.5 Hz), 4.69 (1H, m), 7.04 (2H, d, J=8.5 Hz), 7.27 (2H, d, J=8.5 Hz), 7.60 (2H, t, J=7.6 Hz), 7.69 (1H, t, J=7.4 Hz), 7.86 (2H, d, J=7.4 Hz).

For in vitro and in vivo experiments, BOP was converted to the sodium salt by treatment of a solution of the free acid of BOP in MeOH with 0.98 equivalents of NaOH (0.01 M NaOH). The solution was filtered through a 0.45 μm syringe filter unit and the product lyophilised to give the sodium salt as a fluffy colourless powder. δ_{H} (400 MHz, D₂O) 1.47-1.59 (2H, m), 1.68-1.83 (2H, m), 1.87-1.92 (4H, m), 3.01 (1H, dd, J=7.7, 13.8 Hz), 3.18-3.26 (2H, m), 3.34-3.40 (3H, m), 3.48-3.51 (2H, m), 4.06 (1H, dd, J=4.4, 8.7 Hz), 4.43 (1H, dd, J=5.0, 7.7 Hz), 7.04 (2H, d, J=8.5 Hz), 7.27 (2H, d, J=8.5 Hz), 7.61 (2H, t, J=8.1 Hz), 7.73 (1H, t, J=7.5 Hz), 7.78 (2H, d, J=7.5 Hz).

### Example 1B—Preparation of Fluorescent Labelled Integrin Antagonist with PEG Spacer (Compound 22)

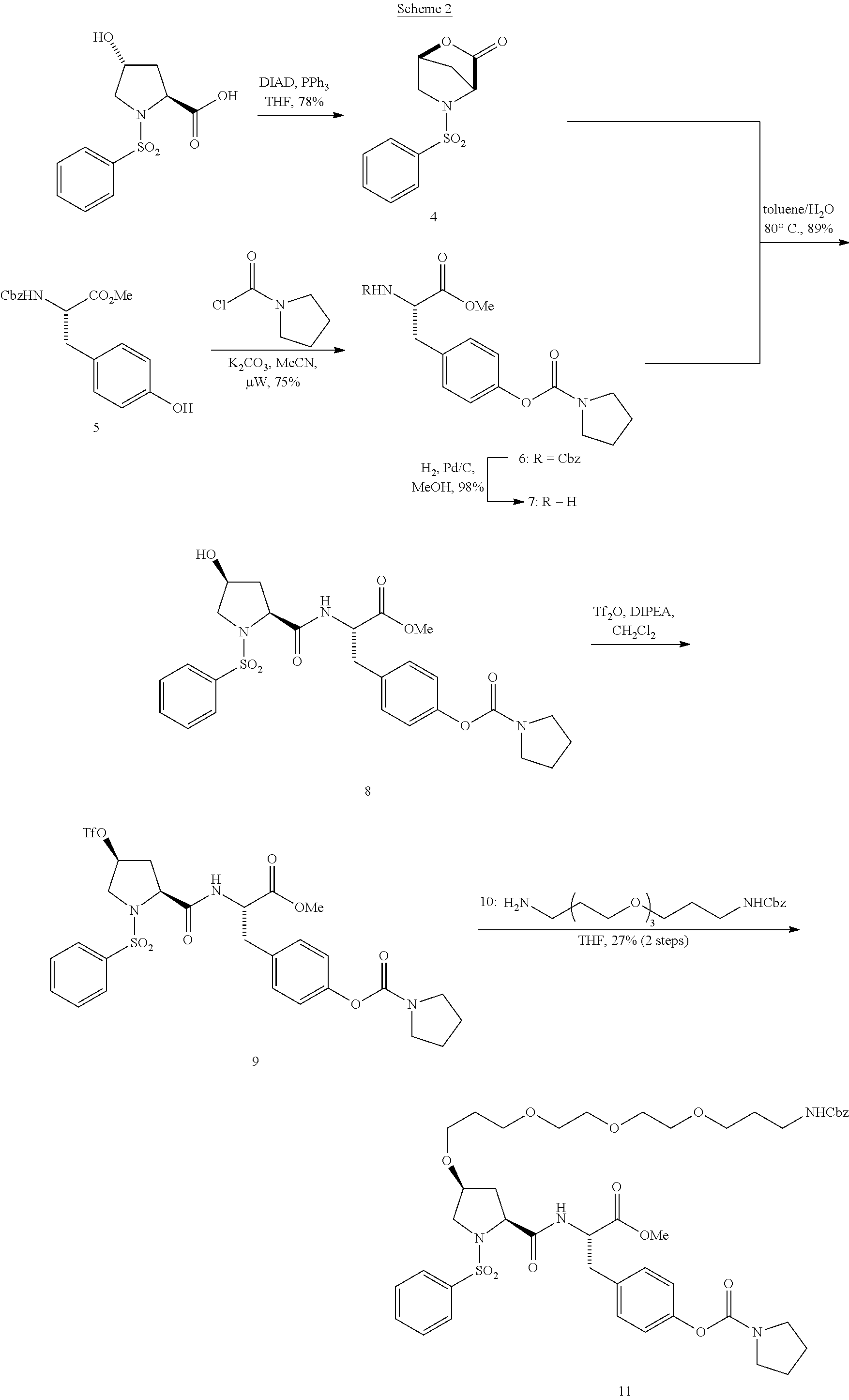

The general strategy for the fluorescent labelling of BOP was based on an efficient strategy for installing a trans-configured bifunctional PEG linker at the C4-position of BOP for subsequent conjugation to a fluorescent tag.

Lactone 4 has previously been reported as a versatile synthon for accessing 4-cis-hydroxy proline based dipeptides through direct acylation with protected amino acids. Subsequent activation of the 4-cis-hydroxy group followed by S_{N}2 displacement with nucleophiles would then provide the desired 4-trans-configured proline derivatives. Thus, we envisaged a variety of C4-functionalised derivatives of BOP could be acquired starting from lactone 4 and tyrosine derivative 7 (Scheme 2). Lactone 4 was readily prepared by treatment of N-phenylsulfonyl-trans-4-hydroxy-L-proline under Mitsunobu conditions employing DIAD and PPh₃. The tyrosine derivative 7 was synthesised from protected 5 by treatment with pyrrolidine carbonyl chloride in the presence of K₂CO₃ to give intermediate 6, followed by removal of the Cbz protecting group. Exposure of lactone 4 to tyrosine derivative 7 under biphasic conditions afforded the dipeptide 8 in 89% yield as a single diastereoisomer. This method takes advantage of the activated nature of the bicyclic lactone and allows clean conversion to the 4-cis-hydroxy proline dipeptides without resorting to dehydrative peptide coupling. Initial attempts in installing the PEG linker focused on the direct displacement of cis-configured triflate 9 with the amino PEG derivative 10, which could be readily obtained from commercially accessible 4,7,10-trioxa-1,13-tridecanediamine. S_{N}2 displacement of 4-cis-triflates with amines has been reported to give the corresponding trans-amino proline derivatives, which would be attractive in the current context given the low steric bulk of the resultant linkage. Accordingly, the hydroxyl group of compound 8 was converted to the corresponding triflate 9 prior to treatment with the PEG derivative 10, which gave the PEGylated product 11, albeit in disappointing yields (27% over 2 steps) (Scheme 2).

Although no major side products were isolated during the formation of either the triflate 9, or the PEG derivative 10, a possible rationalisation for the poor yields of the N-alkylation reaction was intermediate formation of the trifluoromethanesulfonyl imidate. Therefore, the simplified cis-hydroxy compound 12, which lacks a secondary amide was also investigated.

The introduction of N-linked aromatic heterocycles (e.g. imidazoles, triazoles, tetrazoles and benzimidazoles) at the 4-position of the proline residue has previously been described. Based on this observation, we anticipated that attachment of a PEG linker via a triazole might also be tolerated for α₉β₁ integrin binding. Consequently, this would allow installation of the PEG linker using the Cu(I)-catalyzed azide alkyne cycloaddition (CuAAC) reaction between an alkyne functionalised PEG derivative 15 and a trans-azido integrin antagonist 18, as shown in the following Scheme 3:

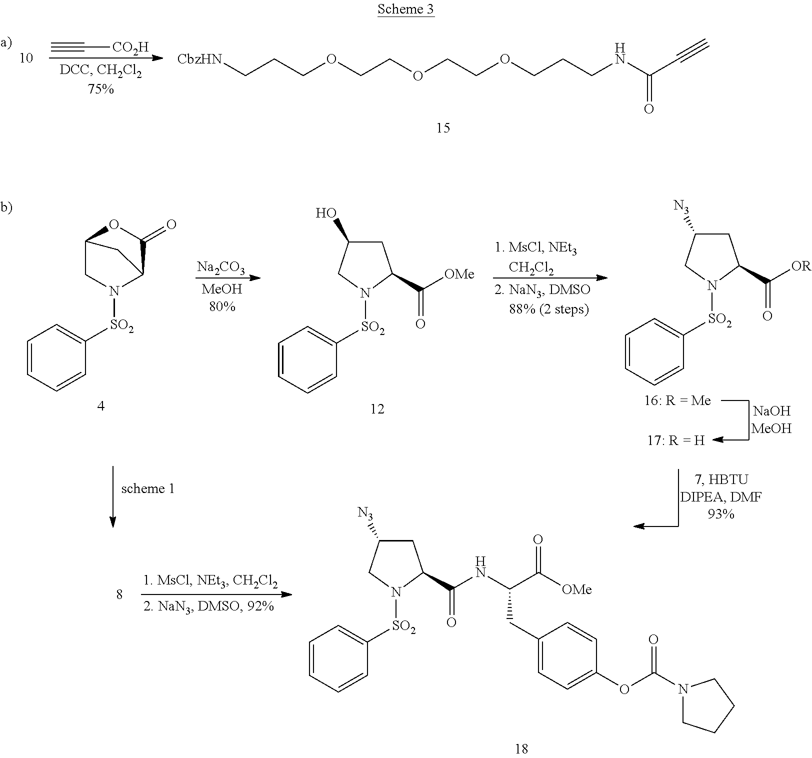

The alkyne functionalised PEG derivative 15 was obtained in one step from 10 by condensation with propionic acid under DCC coupling conditions (Scheme 2a). The synthesis of trans-azido functionalised dipeptide 18 was readily achieved from lactone 4 (Scheme 2b). Treatment of 4 with Nα₂CO₃ in MeOH afforded the cis-hydroxy proline ester 12 in 80% yield. The cis-alcohol of 12 was converted to the corresponding mesylate, which was subsequently displaced with sodium azide to give the trans-azido proline ester 16 in 88% yield over 2 steps. Hydrolysis of the methyl ester of 16 gave the proline acid 17, which was then reacted with the tyrosine derivative 7 under standard HBTU coupling conditions to furnish the dipeptide 18 in 93% yield. Alternatively, dipeptide 18 is also accessible from the cis-alcohol 8 (from Scheme 1). Conveniently, mesylation and subsequent azide displacement of alcohol 8 proceeded smoothly to furnish product 18, which was obtained without the necessity for chromatographic purification.

With the PEG alkyne 15 and azide 18 in hand, attention turned to their coupling using the CuAAC reaction, as shown in the following Scheme 4:

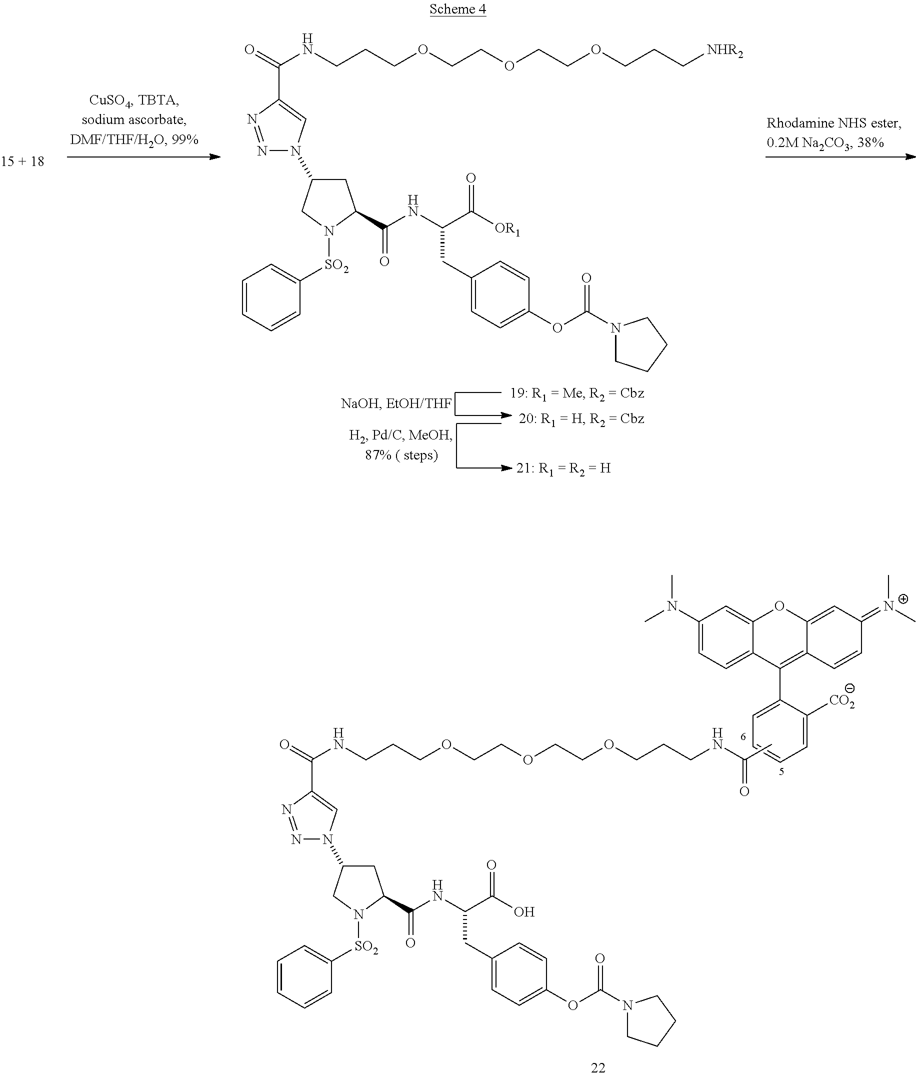

Satisfyingly, treatment of 15 and 18 with CuSO₄, sodium ascorbate and the Cu(I)-stabilising ligand tris[(1-benzyl-1H-1,2,3-triazol-4-yl)methyl]amine (TBTA) gave the 1,4-disubstituted triazole 19 in virtually quantitative yield. Hydrolysis of methyl ester 19 gave the acid 20 and following removal of the Cbz group by hydrogenolysis, the fully deprotected PEG-functionalised integrin antagonist 21 was obtained in good yields (87% over 2 steps). Finally, treatment of amine 21 with NHS-rhodamine under aqueous conditions gave the fluorescent labelled integrin antagonist 22 in 38% yield as a 5:1 mixture of 5- and 6-carboxytetramethylrhodamine regioisomers after purification by C18 reversed phase chromatography.

By way of exemplification we provide actual reaction conditions for the formation of fluorescently labelled BOP derivative 22, starting from N-phenylsulfonyl-trans-4-hydroxy-L-proline.

### Step 1: (1S,4S)-5-(Phenylsulfonyl)-2-oxa-5-azabicyclo[2.2.1]heptan-3-one (4)

Diisopropylazocarboxylate (1.87 mL, 9.48 mmol) was added dropwise over 20 min to a stirred suspension of N-phenylsulfonyl-trans-4-hydroxy-L-proline (2.45 g, 9.03 mmol) and triphenylphosphine (2.49 g, 9.48 mmol) in CH₂Cl₂ (150 mL) at 0° C. under N₂. The reaction was warmed to rt and stirred overnight, concentrated under reduced pressure and the residue purified by flash chromatography (50:50 EtOAc/pet. spirits) to give the lactone 4 (1.77 g, 78%) as a colourless solid. Spectroscopic data is identical to previously reported values.

### Step 2: (S)-4-(2-(((Benzyloxy)carbonyl)amino)-3-methoxy-3-oxopropyl)phenyl pyrrolidine-1-carboxylate (6)

1-Pyrrolidinecarbonyl chloride (336 μL, 3.04 mmol) was added to a Biotage™ microwave vial containing a mixture of the tyrosine 5 (500 mg, 1.52 mmol) and K₂CO₃ (420 mg, 3.04 mmol) in CH₃CN (9 mL). The mixture was heated to 100° C.in a microwave reactor for 45 min, diluted with H₂O and then stirred for 30 min. The aqueous layer was extracted with EtOAc (2×20 mL) and the combined organic phases washed with sat. aq. NaHCO₃, dried (MgSO₄) and concentrated under reduced pressure. The residue was recrystallised (EtOAc/pet. spirits) to give the carbamate 6 (484 mg, 75%) as colourless crystals, mp 116-117° C.; [α]_{D} +42.5 (c 0.81 in CHCl₃); δH (400 MHz, CDCl₃) 1.94 (4H, m, (CH₂)₂), 3.09 (2H, m), 3.47 (2H, t, J=6.5 Hz), 3.55 (2H, t, J=6.5 Hz), 3.71 (3H, s), 4.64 (1H, m), 5.10 (2H, s), 5.22 (1H, d, J=8.0 Hz), 7.03-7.38 (9H, m); δC (100 MHz, CDCl₃) 25.0, 25.8, 37.5, 46.3, 46.4, 52.3, 54.8, 67.00, 121.9 (2 C), 128.1 (2 C), 128.1, 128.5 (2 C), 130.0 (2 C), 132.4, 136.2, 150.6, 153.0, 155.6, 171.9; v/cm⁻¹ 3331, 2954, 1719, 1698; 1511, 1402, 1345, 1216, 1061, 1020, 866, 754, 699; HRMS (ESI+) m/z 449.1686 (C₂₃H₂₆N₂NaO₆ [M+Na]⁺ requires 449.1689).

### Step 3: (S)-4-(2-Amino-3-methoxy-3-oxopropyl)phenyl pyrrolidine-1-carboxylate (7)

A mixture of protected tyrosine 6 (950 mg, 1.13 mmol) and Pd/C (10%, 50 mg) in MeOH (40 mL) was purged three times with H₂. The mixture was stirred under a H₂ atmosphere for 2 h at which point TLC indicated complete consumption of starting material. The mixture was filtered through a layer of Celite and the filtrate concentrated under reduced pressure to give the crude amine 7 (637 mg, 98%) as a colourless oil, which set solid upon standing. A small portion was further purified by flash chromatography (5:95 to 10:90 MeOH/CH₂Cl₂) for characterisation, [α]_{D} −11.6 (c 1.09 in MeOH); δ_{H} (400 MHz, CDCl₃) 1.97 (4H, m), 2.91 (1H, dd, J=7.1, 13.6 Hz), 3.02 (1H, dd, J=6.1, 13.6 Hz), 3.42 (2H, t, J=6.5 Hz), 3.43 (2H, t, J=6.5 Hz), 3.68 (3H, s), 4.84 (2H, br s), 3.70 (1H, dd, J=6.2, 7.1 Hz), 7.05 (2H, m), 7.21 (2H, m); δ_{C} (100 MHz, CDCl₃) 25.9, 26.7, 41.1, 47.5, 47.5, 52.4, 56.7, 123.0 (2 C), 131.2 (2 C), 135.6, 151.6, 155.1, 176.1; v/cm⁻¹ 3311, 2954, 2878, 1714, 1510, 1399, 1344, 1214, 1168, 1086, 1062, 1020, 864, 755; HRMS (ESI⁺) m/z 293.1497 (C₁₅H₂₀N₂O₄SH [M+H]⁺ requires 293.1496).

### Step 4: 4-((S)-2-((2S,4S)-4-Hydroxy-1-(phenylsulfonyl)pyrrolidine-2-carboxamido)-3-methoxy-3-oxopropyl)phenyl pyrrolidine-1-carboxylate (8)

The lactone 4 (466 mg, 1.84 mmol) and the amine 7 (510 mg, 1.76 mmol) in toluene/H₂O (5:1, 6 mL) was stirred at 80° C.for 2 d and then diluted with EtOAc and washed with 1 M HCl, sat. aq. NaHCO₃, brine, dried (MgSO₄) and concentrated under reduced pressure. The residue was purified by flash chromatography (100% EtOAc to 5% MeOH/EtOAc) to give the alcohol 8 (851 mg, 89%) as a colourless foam, [α]_{D} −31.4 (c 1.66 in MeOH); δ_{H} (400 MHz, CDCl₃) 1.57 (1H, m), 1.89-2.00 (5H, m), 2.94 (1H, dd, J=9.5, 14.0 Hz), 3.13 (1H, dd, J=4.0, 10.5 Hz), 3.23 (1H, d, J=10.5 Hz), 3.35 (1H, dd, J=5.0, 14.0 Hz), 3.40 (2H, t, J=6.5 Hz), 3.51-3.56 (3H, m), 3.78 (3H, s), 4.03 (1H, m), 4.12 (1H, d, J=9.0 Hz), 4.75 (1H, m), 6.99 (2H, d, J=8.3 Hz), 7.07 (1H, d, J=7.5 Hz), 7.19 (2H, 8.3 Hz), 7.51-7.63 (3H, m), 7.83 (2H, d, J=7.5 Hz); δ_{C} (100 MHz, CDCl₃) 25.1, 25.9, 37.0, 37.6, 46.5, 46.6, 52.6, 53.3, 57.8, 61.7, 69.7, 122.5 (2 C), 127.9 (2 C), 129.4 (2 C), 130.5 (2 C), 133.4, 133.8, 136.3, 150.2, 154.1, 171.6, 171.7; v/cm⁻¹ 3408, 1743, 1718, 1701, 1663; HRMS (ESI⁺) m/z 568.1723 (C₂₆H₃₁NaN₃O₈S [M+Na]⁺ requires 568.1730).

### Step 5: 4-((R)-3-Methoxy-3-oxo-2-((2S,4R)-4-((3-oxo-1-phenyl-2,8,11,14-tetraoxa-4-azaheptadecan-17-yl)amino)-1-(phenylsulfonyl)pyrrolidine-2-carboxamido)propyl) phenyl pyrrolidine-1-carboxylate (11)

Alcohol 8 (105 mg, 0.19 mmol) was dissolved in dry CH₂Cl₂ (2 mL) under N₂ at −20° C. DIPEA (99 μL, 0.57 mmol) was added followed by Tf₂O (50 μL, 0.57 mmol) dropwise over 30 min. The reaction was stirred for 2 h at −20° C.and then quenched with sat. aq. NaHCO₃, diluted with EtOAc and the organic phase separated. The organic phase was washed with H₂O, 2% citric acid, sat. aq. NaHCO₃ and brine. The aqueous phase was extracted with EtOAc (2 times) and the combined organic phases dried (MgSO₄) and the residue concentrated under reduced pressure to give the crude triflate 9. To this residue was added the PEG amine 10 (141 mg, 0.39 mmol) in dry THF (200 μL) and the reaction stirred overnight at rt. The mixture was diluted with 10% butan-2-ol/EtOAc (20 mL) and the organic phase washed with sat. aq. NaHCO₃, brine, dried (MgSO₄) and concentrated under reduced pressure. The crude residue was purified by flash chromatography (2% to 3% MeOH/CH₂Cl₂) to give 11 (46 mg, 27% over 2 steps) as a colourless oil. A small portion was further purified by C18-silica gel chromatography (40% H₂O/MeCN) for characterisation, δ_{H} (400 MHz, CDCl₃) 1.40 (1H, m), 1.55 (2H, m), 1.77 (2H, m), 1.93 (4H, m), 2.12 (1H, m), 2.43 (2H, m), 2.82 (1H, dd, J=7.8, 9.2 Hz), 2.94 (1H, m), 3.02 (1H, dd, J=7.8, 14.0 Hz), 3.23-3.32 (4H, m), 3.38 (2H, t, J=6.1 Hz), 3.44 (2H, t, J=6.6 Hz), 3.46-3.60 (14H, m), 3.76 (3H, s), 4.09 (1H, dd, J=3.0, 8.9 Hz), 4.85 (1H, td, J=5.7, 7.8 Hz), 5.07 (2H, s), 5.42 (1H, brs), 7.05 (2H, d, J=8.6 Hz), 7.14 (2H, d, J=8.6 Hz), 7.21 (1H, d, J=7.8 Hz), 7.28-7.35 (5H, br m), 7.53 (2H, t, J=7.5 Hz), 7.60 (1H, br t, J=7.04), 7.81 (1H, br d, J=7.05 Hz); δ_{C} (100 MHz, CDCl₃) 25.1, 25.9, 29.6, 30.1, 36.5, 37.5, 39.4, 45.9, 46.5, 46.6, 52.6, 53.3, 54.7, 56.5, 61.6, 66.6, 69.8, 69.7, 70.3, 70.3, 70.67, 70.72, 122.0 (2 C), 128.1 (2 C), 128.2 (2 C), 128.6 (3 C), 129.4 (2 C), 130.2 (2 C), 133.0, 133.5, 136.0, 137.0, 150.7, 153.2, 156.6, 170.9, 171.6; v/cm⁻¹ 3334, 2877, 2341, 1706, 1521. HRMS (ESI⁺) m/z 904.3770 (C₄₄H₅₉NaN₅O₁₂S [M+Na]⁺ requires 904.3779).

### Step 6: Methyl (2S,4S)-4-hydroxy-1-(phenylsulfonyl)pyrrolidine-2-carboxylate (12)

A mixture of lactone 4 (1.74 g, 6.88 mmol) and Nα₂CO₃ (3.65 g, 34.4 mmol) was stirred in MeOH (50 mL) at rt overnight. The residue was concentrated, taken up in EtOAc (100 mL), and H₂O and the organic phase separated. The aqueous phase was extracted with EtOAc (2×30 mL) and the combined organic phases washed with brine, dried (MgSO₄) and concentrated under reduced pressure to give the methyl ester 12 (1.57 g, 80%) as a colourless solid. Spectroscopic data is consistent with reported values and the material was used without further purification.

### Step 7: Methyl (2S,4R)-4-azido-1-(phenylsulfonyl)pyrrolidine-2-carboxylate (16)

MsCl (304 μL, 3.93 mmol) was added to a mixture of the alcohol 12 (934 mg, 3.27 mmol) and triethylamine (620 μL, 4.48 mmol) in dry CH₂Cl₂ (20 mL) at 0° C. under N₂. The mixture was stirred for 2 h, diluted with CH₂Cl₂ and washed sequentially with 5% HCl, sat. aq. NaHCO₃, brine, dried (MgSO₄) and concentrated under reduced pressure to give the crude mesylate as a pale yellow oil. This residue was taken up in DMSO (13 ml) and treated with NaN₃ (638 mg, 9.81 mmol) and the mixture stirred overnight at 80° C. The reaction was diluted with EtOAc and washed with H₂O, brine, dried (MgSO₄) and concentrated. The residue was purified by recrystallisation (MeOH) to give 16 (874 mg, 86% over 2 steps) as colourless needles, mp 98-100° C., [α]_{D} −33.4 (c 1.02 in CHCl₃); δ_{H} (400 MHz, CDCl₃) 2.17-2.21 (2H, m), 3.43 (1H, ddd, J=0.7, 3.0, 11.0 Hz), 3.71 (1H, dd, J=5.0, 11.0 Hz), 3.76 (3H, s), 4.18-4.22 (1H, m), 4.30 (1H, t, J=7.5 Hz), 7.53-7.58 (2H, m), 7.61-7.65 (1H, m), 7.87-7.89 (2H, m); δ_{C} (100 MHz, CDCl₃) 36.5, 52.9, 53.2, 59.45, 59.51, 127.6 (2 C), 129.3 (2 C), 133.3, 137.6, 171.9; v/cm⁻¹ 2101, 1747, 1445, 1345, 1207, 1158, 1095, 1017, 758, 722, 696; HRMS (ESI⁺) m/z 311.0808 (C₁₂H₁₄N₄O₄SH [M+H]⁺ requires 311.0809); m/z 328.1073 (C₁₂H₁₄N₄O₄SNH₄ [M+NH₄]⁺ requires 328.1074.

### Step 8: (2S,4R)-4-azido-1-(phenylsulfonyl)pyrrolidine-2-carboxylic acid (17)

The methyl ester 16 (586 mg, 1.89 mmol) in 3:1 EtOH/THF (40 mL) was treated with 0.2 M NaOH (12.3 mL, 2.45 mmol). The mixture was stirred for 3 h at rt and then concentrated under reduced pressure. The crude material was diluted with diethyl ether and the aqueous phase separated. The organic layer was extracted with 0.2 M NaOH (2×10 mL) and the combined aqueous extract was acidified with 10% HCl. The aqueous layer was extracted with CHCl₃ (4×30 mL) and the combined organic phases washed with brine, dried (MgSO₄) and concentrated under reduced pressure. The crude material was purified by flash chromatography (5% MeOH/CH₂Cl₂ with 0.5% AcOH) to give acid 17 (492 mg, 88%) as a colourless oil, [α]_{D} −34.4 (c 0.82 in MeOH); δ_{H} (400 MHz, CDCl₃) 2.18-2.32 (2H, m), 3.40 (1H, m), 3.73 (1H, dd, J=4.8, 11.5 Hz, H5′), 4.22 (1H, m, H4), 4.29 (1H, t, J=7.7 Hz), 7.56 (2H, m), 7.64 (1H, m), 7.88 (2H, m), 10.72 (1H, br s); δ_{C} (100 MHz, CDCl₃) 36.1, 53.4, 59.5, 127.6 (2 C), 129.4 (2 C), 133.6, 136.9, 176.6; v/cm⁻¹ 3500-2500, 2107, 1731; HRMS (ESI⁺) m/z 319.0472 (C₁₁H₁₂N₄NaO₄S [M+Na]⁺ requires 319.0472).

### Step 9: 4-((S)-2-((2S,4R)-4-azido-1-(phenylsulfonyl)pyrrolidine-2-carboxamido)-3-methoxy-3-oxopropyl)phenyl pyrrolidine-1-carboxylate (18)

O-(Benzotriazol-1-yl)-N,N,N′,N′-tetramethyl uronium hexafluorophosphate (HBTU) (693 mg, 1.83 mmol) was added to a stirred mixture of acid 17 (491 mg, 1.66 mmol) and N,N-diisopropylethylamine (DIPEA) (578 μL, 3.32 mmol) in DMF (6 mL) at 0° C. and stirred for 10 min under N₂. Amine 7 (484 mg, 1.66 mmol) in DMF (6 mL) was then added dropwise and the combined mixture warmed to rt and stirred overnight. The mixture was diluted with EtOAc, washed sequentially with 5% HCl, sat. aq. NaHCO₃, brine, dried (MgSO₄) and concentrated under reduced pressure. The residue was purified by flash chromatography (3% MeOH/CH₂Cl₂) to give the dipeptide 7 (928 mg, 98%) as a pale yellow foam, [α]_{D} −4.6 (c 0.85 in CHCl₃); δ_{H} (400 MHz, CDCl₃) 1.67 (1H, m), 1.86-1.98 (4H, m), 2.04 (1H, dt, J=5.5, 13.2 Hz), 2.99 (1H, dd, J=8.5, 14.0 Hz), 3.19 (1H, dd, J=4.5, 10.9 Hz), 3.30 (1H, dd, J=5.5 14.0 Hz), 3.44 (2H, t, J=6.5 Hz), 3.48 (1H, dd, J=5.5, 11.5 Hz), 3.53 (2H, t, J=6.5 Hz), 3.78 (3H, s), 3.82 (1H, m), 4.09 (1H, dd, J=5.5, 8.4 Hz), 4.87 (1H, dt, J=8.3, 8.3, 5.5 Hz), 7.05, 7.15 (4H, 2×d, J=8.5 Hz), 7.19 (1H, d, J=8.2 Hz), 7.53-7.57 (2H, m), 7.62-7.66 (1H, m), 7.83-7.86 (2H, m); δ_{C} (100 MHz, CDCl₃) 25.1, 25.9, 35.6, 37.5, 46.4, 46.6, 52.7, 53.1, 53.8, 58.9, 61.1, 122.1 (2 C), 128.0 (2 C), 129.5 (2 C), 130.2 (2 C), 132.9, 133.7, 136.0, 150.7, 153.1, 169.9, 171.5; v/cm⁻¹ 3316, 2975, 2880, 2105, 1716, 1682; HRMS (ESI⁺) m/z 593.1789 (C₂₆H₃₀N₆NaO₇S [M+Na]⁺ requires 593.1789).

### Synthesis of 18 Via Alcohol 8

Methanesulfonyl chloride (92 μL, 1.18 mmol) was added to a stirred mixture of the alcohol 8 (251 mg, 0.394 mmol) and triethylamine (170 μL, 1.22 mmol) in dry CH₂Cl₂ at 0° C. under N₂. The reaction was stirred for 1 h at 0° C. and then warmed to rt and stirred for a further 1 h. The reaction was diluted with CH₂Cl₂ and washed sequentially with 5% HCl, sat. aq. NaHCO₃ and brine. The organic phase was dried (MgSO₄) and concentrated under reduced pressure to give the intermediate mesylate (4-((S)-3-methoxy-2-((2S,4S)-4-((methylsulfonyl)oxy)-1-(phenylsulfonyl) pyrrolidine-2-carboxamido)-3-oxopropyl)phenyl pyrrolidine-1-carboxylate) (270 mg, quant) as a colourless foam, which was used without further purification, [α]_{D} −13.5 (c 1.0 in CHCl₃); δ_{H} (400 MHz, CDCl₃) 1.81 (1H, m), 1.89-1.96 (4H, m), 2.63 (1H, br d), 2.82 (3H, s), 3.06-3.18 (2H, m), 3.44 (2H, t, J=6.4 Hz), 3.50-3.55 (3H, m), 3.68 (1H, dt, J=1.3, 12.5 Hz), 3.71 (3H, s), 4.63 (1H, dd, J=2.0, 10.1 Hz), 4.73 (1H, dd, J=6.7, 13.4 Hz), 5.02 (1H, tt, J=1.4, 4.7 Hz), 7.07 (2H, d, J=8.6), 7.16 (2H, d, J=8.6 Hz), 7.39 (1H, d, J=7.5 Hz), 7.56 (2H, t, J=7.6 Hz), 7.64-7.68 (1H, m), 7.81-7.84 (2H, m); δ_{C} (100 MHz, CDCl₃) 25.1, 25.9, 35.5, 37.4, 38.9, 46.5, 46.5, 52.5, 54.0, 55.4, 61.0, 78.0, 122.1 (2 C), 128.0 (2 C), 129.8 (2 C), 130.1 (2 C), 132.8, 134.1, 135.4, 150.7, 153.1, 169.8, 171.3; v/cm⁻¹ 3409, 2954, 2880, 1715, 1678, 1511; HRMS (ESI⁺) m/z 646.1497 (C₂₇H₃₃NaN₃O₁₀S₂[M+Na]⁺ requires 646.1505).

The above mesylate (97 mg, 0.16 mmol) was taken up in DMSO (1.5 mL) and treated with NaN₃ (30.4 mg, 0.47 mmol) and the mixture stirred overnight at 80° C. The reaction was diluted with EtOAc and washed with H₂O, brine, dried (MgSO₄) and concentrated to give azide 18 (82 mg, 92%). Spectroscopic data were consistent with those reported above for compound 18.

### Step 10: 4-((R)-3-methoxy-3-oxo-2-((2S,4R)-4-(4-((3-oxo-1-phenyl-2,8,11,14-tetraoxa-4-azaheptadecan-17-yl)carbamoyl)-1H-1,2,3-triazol-1-yl)-1-(phenylsulfonyl) pyrrolidine-2-carboxamido)propyl)phenyl pyrrolidine-1-carboxylate (19)

Sodium ascorbate (4.4 mg, 22.2 μmol), CuSO₄ (224 μL, 2.24 μmol, 0.01 M in H₂O) and TBTA (281 μL, 2.81 μmol, 0.01 M in THF) were added sequentially to a mixture of the azide 18 (32 mg, 56.1 μmol) and the alkyne 15 (25 mg, 61.8 μmol) in DMF (1 mL). The reaction was stirred at 60° C. for 2 h and then diluted with EtOAc (20 mL). The organic phase was washed with sat. aq. NaHCO₃, brine, dried (MgSO₄) and concentrated under reduced pressure. The residue was purified by flash chromatography (21:2:1:1 EtOAc/acetone/MeOH/H₂O) to give the triazole product 19 (54 mg, 99%) as a colourless oil, [α]_{D} +10.7 (c 1.0 in CHCl₃); δ_{H} (400 MHz, CDCl₃) 1.72-1.94 (8H, m), 2.14 (1H, dt, J=12.8, 13.9 Hz), 2.54 (1H, ddd, J=2.3, 6.5, 12.9 Hz), 2.92 (1H, dd, J=10.0, 13.9 Hz), 3.29 (2H, dd, J=6.0, 12.3 Hz), 3.38 (1H, dd, J=4.8 Hz, 13.9 Hz), 3.41-3.63 (19H, m), 3.80 (3H, s), 3.83 (1H, m), 4.29 (1H, dd, J=2.0, 8.7 Hz), 4.65 (1H, m), 4.94 (1H, dt, J=4.9, 9.8 Hz), 5.06 (2H, br s), 5.44 (1H, br t, J=5.7 Hz), 7.04 (2H, d, J=8.5 Hz), 7.23 (2H, d, J=8.5 Hz), 7.28-7.33 (5H, m), 7.38-7.43 (2H, m), 7.52 (2H, t, J=7.7 Hz), 7.62 (1H, t, J=7.5 Hz), 7.79 (2H, d, J=7.3 Hz), 8.18 (1H, s); δ_{C} (100 MHz, CDCl₃) 25.1, 25.9, 29.4, 29.5, 33.7, 37.2, 37.9, 39.4, 46.4, 46.6, 52.7, 53.2, 53.6, 57.9, 60.7, 66.5, 69.7, 69.7, 70.3, 70.5, 70.6, 70.7, 122.2 (2 C), 126.1, 127.7 (2 C), 128.1, 128.2, 128.5 (3 C), 129.7 (2 C), 130.3 (2 C), 133.1, 133.9, 135.5, 136.9, 143.2, 150.6, 153.3, 156.6, 159.8, 169.0, 171.5; v/cm⁻¹ 3331, 2951, 2875, 1714, 1667, 1575, 1512; HRMS (ESI⁺) m/z 999.3891 (C₄₇H₆₀NaN₈O₁₃S [M+Na]⁺ requires 999.3893).

### Step 11: (R)-2-((2S,4R)-4-(4-((3-(2-(2-(3-aminopropoxy)ethoxy)ethoxy)propyl) carbamoyl)-1H-1,2,3-triazol-1-yl)-1-(phenylsulfonyl)pyrrolidine-2-carboxamido)-3-(4-((pyrrolidine-1-carbonyl)oxy)phenyl)propanoic acid (21)

Aqueous NaOH (1.13 mL, 0.225 mmol, 0.2 M) was added to a stirred mixture of methyl ester 19 (110 mg, 0.113 mmol) in EtOH/THF (2:1, 3 mL) and stirred overnight at rt. The reaction was quenched with 1 M HCl, diluted with EtOAc and the organic phase separated. The aqueous phase was extracted twice with CHCl₃ and the combined organic phases dried (MgSO₄) and concentrated under reduced pressure to give the crude acid 20 (100 mg). A mixture of the crude acid and 10% Pd/C (50 mg, 50% H₂O) in MeOH/H₂O (5:1, 12 mL) was stirred under a H₂ atmosphere for 2 h at rt. The mixture was filtered through a layer of Celite, concentrated under reduced pressure and the residue purified using a C18 reversed phase cartridge (100% H₂O to 50% MeOH/H₂O). The purified material was lyophilised to give the amine 21 (81.3 mg, 87% over 2 steps) as a colourless fluffy powder, [α]_{D} −16.0 (c 0.49 in MeOH); δ_{H}(400 MHz, D₂O) 1.81 (4H, m), 1.91 (4H, m), 2.38 (1H, m), 2.98-3.09 (4H, m), 3.21-3.30 (3H, m), 3.30 (2H, m), 3.45 (2H, t, J=6.8 Hz), 3.59-3.66 (12H, m), 3.89 (1H, d, J=12.9 Hz), 4.04 (1H, dd, J=4.8, 12.9 Hz), 4.41 (1H, t, J=8.2 Hz), 4.53 (1H, dd, J=5.3, 7.4 Hz), 5.00 (1H, br s), 6.99 (2H, d, J=8.5 Hz), 7.306-7.356 (4H, m), 7.43-7.50 (3H, m), 7.99 (1H, s); δ_{C} (100 MHz, D₂O with acetone) 25.2, 25.8, 27.2, 29.1, 35.5, 36.9, 37.2, 38.3, 47.1, 47.1, 49.5, 55.1, 56.7, 60.4, 61.7, 68.9, 69.3, 70.1, 70.2, 70.3, 122.6 (2 C), 125.8, 127.5 (2 C), 130.2 (2 C), 131.2 (2 C), 134.5, 135.1, 135.6, 142.9, 150.4, 155.6, 161.9, 173.2, 175.8; v/cm⁻¹ 3382, 3064, 2950, 2878, 1706, 1658, 1511; HRMS (ESI⁺) m/z 829.3550 (C₃₈H₅₂N₈O₁₁S [M+H]⁺ requires 829.3549).

### Step 12: 5(6)-Carboxytetramethyl rhodamine labelled compound (22)

A mixture of the amine 21 (9.5 mg, 11.3 μmol) in 0.2 M NaHCO₃ (1 mL) was treated with 5(6)-carboxytetramethyl rhodamine N-succinimidyl ester (NHS-rhodamine, Thermo Scientific) (8.9 mg, 16.9 μmol) and the mixture was allowed to stir overnight at rt. The reaction was quenched with acetic acid, concentrated and the residue purified by reversed phase chromatography (50% MeOH/H₂O to 100% MeOH) to give the rhodamine labelled compound 22 (5.3 mg, 38%) as a purple powder after lyophilisation. Compound 22 was isolated as a 5:1 mixture of regioisomers; δ_{H} (400 MHz, d₄-methanol) major isomer: 1.80-1.99 (9H, m), 2.37 (1H, dt, J=6.6, 13.5 Hz), 2.70 (1H, m), 3.08 (1H, dd, J=7.5, 13.9 Hz), 3.22-3.26 (1H, m), 3.26 (6H, s), 3.27 (6H, s), 3.35 (2H, t, J=6.3 Hz), 3.43 (2H, t, J=6.3 Hz), 3.48-3.66 (17H, m), 3.74 (1H, dd, J=3.7, 12.0 Hz), 3.91 (1H, dd, J=6.0, 12.0 Hz), 4.45 (1H, t, J=7.1 Hz), 4.61 (1H, t, J=5.6 Hz), 4.94 (1H, m), 6.86 (2H, br s), 6.95-6.99 (4H, m), 7.16 (2H, d, J 30=9.5 Hz), 7.28 (2H, d, J=8.1 Hz), 7.35-7.42 (3H, m), 7.51 (1H, t, J=7.3 Hz), 7.60-7.63 (2H, m), 8.06-8.08 (2H, m), 8.56 (1H, s); δ_{C} (100 MHz, d₄-methanol) major isomer: 25.9, 26.7, 30.4, 36.6, 38.0, 38.1, 38.9, 40.9 (4 C), 47.47, 47.53, 55.9, 60.3, 62.3, 70.3, 70.5, 71.35, 71.4, 71.6 (2 C), 97.3 (2 C), 114.8 (2 C), 115.2 (2 C), 122.7 (4 C), 126.3, 128.6 (2 C), 130.0, 130.1, 130.5 (2 C), 131.1, 131.7 (4 C), 132.5 (2 C), 134.4 (2 C), 136.0, 137.2, 137.3, 138.1, 144.0, 151.6, 155.1, 158.7 (2 C), 159.0 (2 C), 161.6, 162.0, 168.7, 172.6; HRMS (ESI⁺) m/z 1241.4970 (C₆₃H₇₂N₁₀O₁₅SH [M+H]⁺ requires 1241.4972).

### Example 1C—Preparation of R-BC154 (Compound 25)

Compound 25 (R-BC154), which lacks the PEG-spacer was also synthesised, as shown in the following Scheme 5:

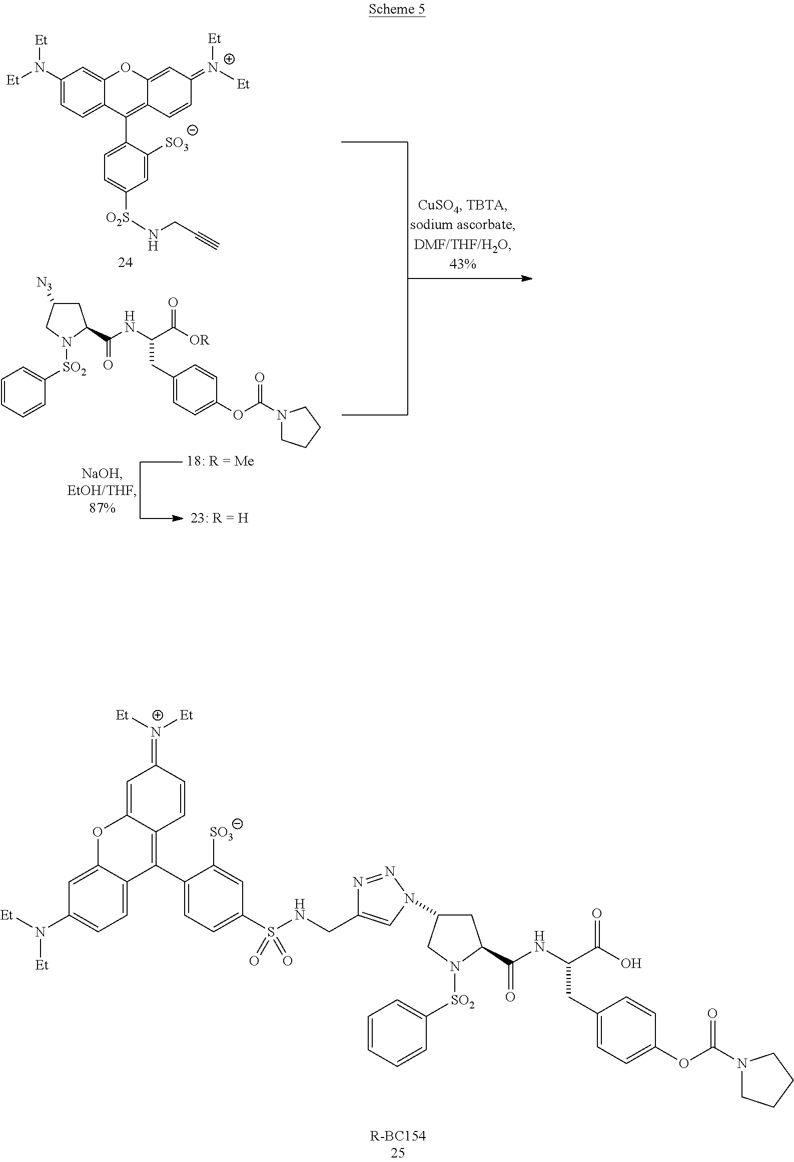

Thus, hydrolysis of the methyl ester 18 with NaOH gave the deprotected azide inhibitor 23, which was subsequently reacted with N-propynyl sulforhodamine B 24 in the presence of CuSO₄, sodium ascorbate and TBTA to give the fluorescent labelled (R-BC154) in 43% yield after purification by HPLC (Scheme 4).

By way of exemplification we provide actual reaction conditions for the formation of fluorescently labelled BOP derivative 25, starting from methyl ester 18.

### Step 1: (S)-2-((2S,4R)-4-Azido-1-(phenylsulfonyl)pyrrolidine-2-carboxamido)-3-(4-((pyrrolidine-1-carbonyl)oxy)phenyl)propanoic acid (23)

The methyl ester 18 (420 mg, 0.737 mmol) in EtOH (10 mL) was treated with 0.2 M NaOH (4.05 mL, 0.811 mmol) and stirred at rt for 1 h. The mixture was concentrated under reduced pressure to remove EtOH and the aqueous phase acidified with 10% HCl. The aqueous phase was extracted with CHCl₃ (4×10 mL) and the combined organic phases were washed with brine, dried (MgSO₄) and concentrated under reduced pressure. The crude material was purified by flash chromatography (10% MeOH/CH₂Cl₂ with 0.5% AcOH) to give acid 23 (384 mg, 94%) as a pale yellow foam, [α]_{D} −0.7 (c 1.00 in CHCl₃); δ_{H} (400 MHz, CDCl₃) 1.67-1.73 (1H, m), 1.89-1.96 (5H, m), 3.10 (1H, dd, J=8.0, 13.8 Hz), 3.21 (1H, dd, J=4.0, 11.5 Hz), 3.38 (1H, dd, J=5.3, 14.0 Hz), 3.44-3.55 (5H, m), 3.81 (1H, m), 4.11 (1H, t, J=6.5 Hz), 4.89 (1H, m), 7.05, 7.22 (4H, 2×d, J=8.0 Hz), 7.41 (1H, d, J=6.8 Hz), 7.53-7.64 (3H, m), 7.85 (2H, d, J=7.5 Hz); δ_{C} (100 MHz, CDCl₃) 25.0, 25.8, 36.1, 36.8, 46.5, 46.6, 53.2, 53.9, 58.9, 61.2, 122.0 (2 C), 128.0 (2 C), 129.4 (2 C), 130.5 (2 C), 133.6, 133.7, 136.0, 150.5, 153.6, 170.9, 173.7; v/cm⁻¹ 3329, 2977, 2881, 2105, 1706, 1672; HRMS (ESI⁺) m/z 557.1817 (C₂₅H₂₉N₇O₆S [M+H]⁺ requires 557.1813).

### Step 2: R-BC154 (25)

The azide 23 (12 mg, 22 μmol) and N-propynyl sulforhodamine B 24 (14 mg, 24 μmol) in DMF (2 mL) were treated with CuSO₄ (86 μL, 0.86 μmol, 0.01 M in H₂O), sodium ascorbate (430 μL, 4.3 μmol, 0.01 M in H₂O) and tris[(1-benzyl-1H-1,2,3-triazol-4-yl)methyl]amine (TBTA) (108 μL, 1.08 μmol, 0.01 M in DMF). The mixture was stirred at 60° C. for 2 h at which point TLC indicated formation of a new fluorescent product. The mixture was concentrated under reduced pressure and the residue partly purified by flash chromatography (40:10:1 CHCl₃/MeOH/H₂O with 0.5% AcOH). This material was further purified by HPLC (50%-98% MeCN/H₂O (0.1% TFA) gradient over 15 minutes; Rₜ=14.9 min) to give pure 25 (10.6 mg, 43%) as a purple glass, 5H (400 MHz, d₄-methanol) 1.27-1.31 (12H, dt, J=7.0, 3.5 Hz), 1.91-1.98 (4H, m), 2.29-2.35 (1H, m), 2.71-2.78 (1H, m), 3.08 (1H, dd, J=7.5, 13.8 Hz), 3.22 (1H, dd, J=5.3, 13.8 Hz), 3.41 (2H, t, J=6.5 Hz), 3.54 (2H, t, J=6.5 Hz), 3.63-3.70 (8H, m), 3.85 (1H, dd, J=3.5, 12.0 Hz), 3.97 (1H, dd, J=5.6, 11.6 Hz), 4.21 (2H, d, J=1.4 Hz), 4.41 (1H, t, J=7.3 Hz), 4.72 (1H, dd, J=5.4, 7.5 Hz), 5.08 (1H, m), 6.91 (2H, t, J=2.2 Hz), 6.98-7.04 (4H, m), 7.11 (2H, t, J=9.0 Hz), 7.30 (2H, d, J=8.6 Hz), 7.40 (1H, d, J=8.0 Hz), 7.44 (2H, t, J=7.5 Hz), 7.58-7.68 (4H, m), 8.00 (1H, dd, J=1.9, 8.0 Hz), 8.37 (1H, d, J=1.8 Hz); δC (100 MHz, d₄-methanol) 12.9 (4 C), 25.9, 26.7, 37.1, 37.6, 39.0, 46.8 (4 C), 47.5, 47.6, 54.8, 55.7, 60.3, 62.1, 97.0 (2 C), 115.0 (2 C), 115.26, 115.29, 122.9 (2 C), 123.9, 127.5, 128.6 (2 C), 129.3, 130.5 (2 C), 131.6 (2 C), 132.3, 133.8, 133.9, 134.4, 135.26, 135.34, 138.3, 144.1, 144.8, 146.9, 151.7, 155.2, 157.16, 157.17, 157.2, 157.8, 159.4, 173.1, 173.8; v/cm⁻¹ 3088-3418, 2977, 2876, 1711, 1649, 1588; HRMS (ESI⁺) m/z 1174.3447 (C₅₅H₆₁N₉NaO₁₃S₃[M+Na]⁺ requires 1174.3443). For in vitro and in vivo testing, the free acid of 25 (11.7 mg, 9.97 pmol) was dissolved in 0.01 M NaOH (997 μL, 9.97 μmol) and the dark purple solution filtered through a 0.45 μm syringe filter unit. The product was lyophilised to give the sodium salt of 25 (11.6 mg, 99%) as a fluffy purple powder.

### Example 2: In Vitro Binding Properties of Compounds 22 and 25 (R-BC154) to α₄β₁ and α₉β₁

For assessing R-BC154 binding on sorted populations of progenitor cells (LSK cells), whole bone marrow was harvested from untreated and treated (R-BC154; 10 mg kg-1) mice (3 mice per group). Lineage positive cells were immunolabelled using a lineage cocktail (B220, Gr-1, Mac-1 and Ter-119) and then removed by immunomagnetic selection with sheep anti-rat conjugated Dynabeads (Invitrogen) according to the manufacturer's instructions. The resultant lineage depleted cells were stained with anti-Sca-1-PB and anti-c-kit-FITC. Immunolabelled cells were sorted on Sca-1+c-kit+ using a Cytopeia Influx (BD Biosciences) cell sorter and imaged using an Olympus BX51 microscope.

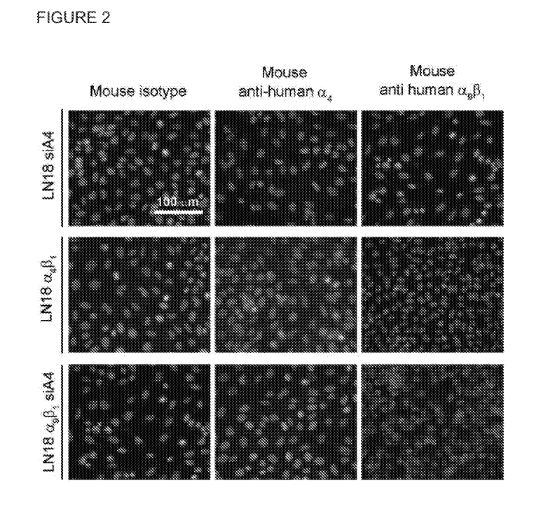

With fluorescent probes compounds 22 and 25 (R-BC154) in hand, the integrin dependent cell binding properties were assessed using α₄β₁ and α₉β₁ over-expressing human glioblastoma LN18 cell lines that were generated. In short, stable LN18 cells over-expressing integrin α₄β₁ and α₉β₁ were generated via retroviral transduction of human glioblastoma LN18 cell lines. Silencing of background α₄ expression in parental and α₉β₁ transduced LN18 cells was achieved by retroviral vector delivery of α₄ shRNA (J Grassinger, et al Blood, 2009, 114, 49-59). (See FIGS. 1 and 2).

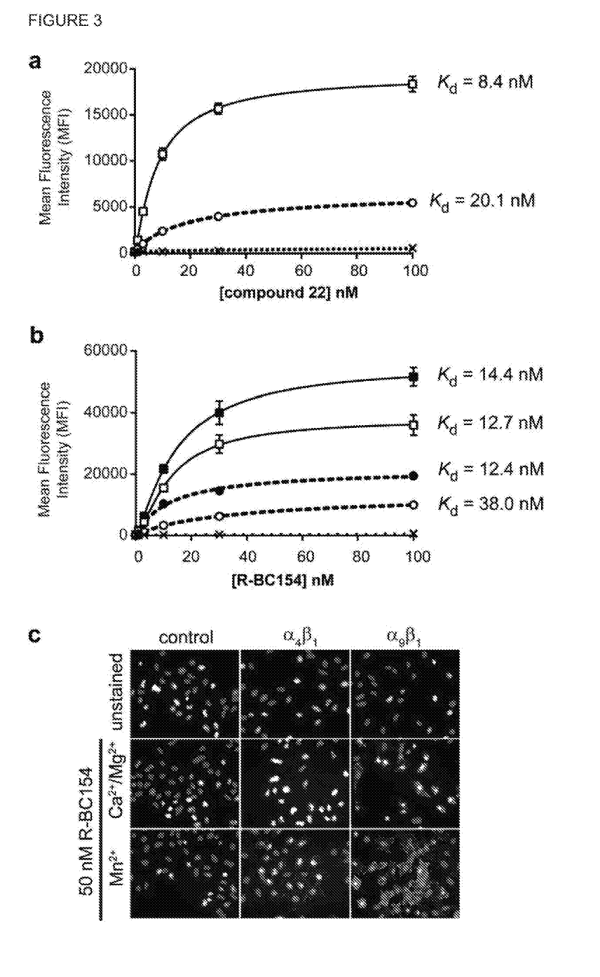

When each LN18 cell line was treated with compounds 22 and R-BC154 (25) under physiological mimicking conditions (1 mM Ca²⁺/Mg²⁺), both compounds were found to bind α₄β₁ and α₉β₁ LN18 cells in a dose-dependent manner (FIGS. 3a and b). Virtually no binding was observed in the control cell line, which lacks integrin expression indicating that binding is integrin specific (FIGS. 3a and b). Both the PEG-linked compound 22 and R-BC154 (25) bound α₉β₁ integrin with greater selectivity than α₄β₁ integrin as determined by their calculated dissociation constants (K_{d}). Specifically, compound 22 binds α₉β₁ (K_{d}=8.4 nM) with 2.4-fold greater affinity than α₄β₁ (K_{d}=20.1 nM) and R-BC154 has 3 times greater affinities for α₉β₁ (K_{d}=12.7 nM) relative to α₄β₁ (K_{d}=38.0 nM) under Ca²⁺/Mg²⁺conditions (FIGS. 3a and b).

Interestingly, when compared to compound 22, R-BC154 was associated with only a 1.9-fold and 1.5-fold reduction in binding affinity to α₄β₁ and α₉β₁ integrins, respectively.

These results suggest that both α₄β₁ and α₉β₁ integrins can indeed tolerate significant steric encumbrances at the 4-position of the proline residue for this class of N-phenylsulfonyl proline-based integrin antagonists. This observation indicates that there may be minimal benefits for the incorporation of a PEG linker.

The surprisingly high affinities of R-BC154 to α₉β₁/α₄β₁ integrins prompted further exploration of its binding properties. Like many integrin ligands, the affinity and binding kinetics of R-BC154 is also dependent on the activation state of integrins, which can be regulated by divalent metal cations. As expected, no integrin binding was observed in the absence of cations (FIG. 4). However, in the presence of 1 mM Mn²⁺, conditions known to induce integrins to adopt a higher affinity binding confirmation, greater overall binding was observed to both α₄β₁ and α₉β₁ over-expressing LN18 cell lines (FIGS. 3b and c). Additionally, under Mn²⁺ activation a 3.1-fold increase in the binding affinity of R-BC154 towards α₄β₁ (K_{d}=12.4 nM) was observed when compared to Ca²⁺/Mg²⁺ conditions (K_{d}=38.0 nM) (FIG. 3b). Despite the greater overall level of α₉β₁ integrin binding that was induced by the addition of Mn²⁺, a minimal change in the binding affinity was evident when compared to Ca²⁺/Mg²⁺ conditions (Kd=14.4 nM vs. 12.7 nM, respectively) (FIG. 3b).

The differences in the biochemical properties of α₄β₁ and α₉β₁ integrins were further investigated by measuring the kinetics of R-BC154 binding. Association rate measurements showed R-BC154 binding under Ca²⁺/Mg²⁺ conditions was faster relative to Mn²⁺conditions for both α₄β₁ and α₉β₁ integrins (FIGS. 5a and b, respectively). Calculation of the on-rate constants (kₒₙ) showed R-BC154 binding to α₄β₁ integrin (kₒₙ=0.094 nM-1 min⁻¹) was faster than α₉β₁ binding (kₒₙ=0.061 nM-1 min-1) under physiological conditions (Table 3). Nevertheless, similar kₒₙ values were observed under Mn²⁺ activation for both α₄β₁ (kₒₙ=0.038 nM-1 min-1) and α₉β₁ integrins (kₒₙ˜0.04 nM-1 min-1) (Table 3).

The off-rate kinetics of R-BC154 binding was determined by dissociation experiments. Under both Ca²⁺/Mg²⁺ and Mn²⁺ states, dissociation rates were faster for the R-BC154-α₄β₁ complex (k_{off}=0.717 and 0.014 min-1) compared to its α₉β₁ (k_{off}=0.054 and <0.01 min-1) counterpart (FIG. 5c and Table 3). In addition, the k_{off} values for R-BC154 binding to α₄β₁ and α₉β₁ integrins in the presence of Mn²⁺ was significantly slower compared to Ca²⁺/Mg²⁺ conditions, with greater than 60% of R-BC154 still bound after 60 min (FIG. 5c). The slower off-rates observed under these conditions suggests Mn²⁺ acts to stabilise the ligand bound conformation and is consistent with previous reports using radiolabelled substrates. Thus, while faster on-rates and off rates are observed with Ca²⁺/Mg²⁺ conditions, Mn²⁺ activation is associated with slower on- and off-rates for α₄β₁ and α₉β₁ integrin binding. Consequently, competitive inhibition assays using R-BC154 for in vitro screening of small molecule integrin inhibitors under Ca²⁺/Mg²⁺conditions is preferred as the exceedingly slow off rates under Mn²⁺ activation would require much longer incubation time.

These results suggest that although α₄β₁ integrin binds slightly faster to this class of N-phenylsulfonyl proline-based antagonists compared to α₉β₁, more prolonged binding is observed for α₉β₁ integrin (Table 3). Thus, under physiologically relevant conditions, this class of dual α₉β₁/α₄β₁ integrin inhibitors might be expected to elicit greater affects against α₉β₁ integrin-dependent inter-actions in vivo owing to their significantly slower off-rates to α₉β₁ despite the higher association rates observed for α₄β₁ integrin.

TABLE 3
Summary of R-BC154 binding properties to α₄β₁
and α₉β₁ overexpressing LN18 cells in the presence
of Ca²⁺/Mg²⁺ or Mn²⁺Conditions

| | | ^{a}k_{obs} | ^{b}k_{off} | ^{e}kₒₙ |
|---|---|---|---|---|
| | Conditions | (min⁻¹) | (min⁻¹) | (nM⁻¹min⁻¹) |
| α₄β₁ cells | 1 mM Ca²⁺/Mg²⁺ | 5.426 | ^{c}0.717 | 0.094 |
| | 1 mM Mn²⁺ | 1.891 | 0.014 | 0.038 |
| α₉β₁ cells | 1 mM Ca²⁺/Mg²⁺ | 3.117 | 0.054 | 0.061 |
| | 1 mM Mn²⁺ | 2.035 | ^{d}<0.01 | ~0.04 |

^{a}The observed association rate (k_{obs}) represents the fast phase of binding and accounts for >60% and >80% of R-BC154 binding to α₄β₁ and α₉β₁ integrins, respectively.
^{b}Data from the dissociation experiment represented in FIG. 5c was fitted to a one-phase exponential decay function (unless otherwise stated) and dissociation rate constants (k_{off}) extrapolated from the curve.
^{c}Dissociation data for R-BC154 binding to α₄β₁ LN18 cells in the presence of Ca²⁺/Mg²⁺ was fitted to a two-phase dissociation curve and k_{off} was determined from the fast-phase of the curve, which accounted for >60% of the dissociation.
^{d}Dissociation of the R-BC154- α9β1 integrin complex under Mn²⁺ activation was too slow (>55% still bound after 120 min; data not shown) to accurately calculate off-rates; k_{off} value was estimated based on an approximate half-life of ″100 min.
^{e}The association rate constant (kₒₙ) was calculated using the formula (k_{obs} − k_{off})/[R-BC154 concentration = 50 nM].

### Example 3: In Vivo Binding of Compound 25 (R-BC154) to Bone Marrow HSC and Progenitor Cells

The in vitro binding data demonstrated that R-BC154 is a high affinity α₄β₁ and α₉β₁ integrin antagonist, whose binding activity is highly dependent on integrin activation. This example tests whether R-BC154 could be used in in vivo binding experiments to investigate α₉β₁/α₄β₁ integrin activity on defined populations of HSC. To date, assessing integrin activity on HSC has relied primarily on in vitro or ex vivo staining of bone marrow cells or purified HSC using fluorescent labelled antibodies. Whilst ex vivo staining provides confirmation of integrin expression by HSC, investigation of integrin activation in their native state within bone marrow can only be determined through in vivo binding experiments, as the complex bone marrow microenvironment cannot be adequately reconstructed in vitro.

To assess whether R-BC154 and this class of N-phenylsulfonyl proline-based peptidomimetics could bind directly to HSC, R-BC154 (10 mg kg⁻¹) was injected intravenously into mice and analysed for R-BC154 labelling of phenotypically defined bone marrow progenitor cells (LSK cell; lineage-Sca-1+c-Kit+) and HSC (LSKSLAM cell; LSKCD48-CD150+) using multi-colour flow cytometry (FIGS. 6a and b). Increased cell-associated fluorescence as a result of R-BC154 binding was observed for both progenitor cells and HSC populations that were isolated from R-BC154 injected mice when compared to bone marrow from un-injected mice. Furthermore, in vivo R-BC154 binding was also confirmed by fluorescence microscopy on purified populations of progenitor cells (Lineage-Sca-1+c-Kit+) (FIGS. 6c and d). R-BC154 labelled progenitor cells exhibited a fluorescence halo indicating R-BC154 binding was primarily cell surface, which is consistent with integrin-binding. The in vivo binding results indicate that this class of α₉β₁/α₄β₁ integrin antagonists are capable of binding to extremely rare populations of haemopoietic progenitor cells and HSC, which represent only 0.2% and 0.002% of mononucleated cells within murine bone marrow, respectively.

The α₄β₁ and α₉β₁ integrins are recognised to be important modulators of HSC lodgement within bone marrow through binding to VCAM-1 and Opn. (J. Grassinger et al, Blood, 2009, 114, 49-59). BOP has been shown to inhibit binding of α₄β₁ and α₉β₁ integrins to both VCAM-1 and Opn in vitro with nanomolar inhibitory potencies. These in vivo binding results using R-BC154 indicate that the α₄β₁ and α₉β₁ integrins expressed by HSC are in an active binding conformation in situ. This suggests that small molecule α₉β₁/α₄β₁ integrin antagonists such as compounds 22 and 25, not only bind directly to bone marrow HSC, but they are also be capable of inhibiting α₉β₁/α₄β₁ dependent adhesive interactions and potentially serve as effective agents for inducing the mobilisation of bone marrow HSC into the peripheral circulation as shown below.

### Example 4—R-BC154 Binds Preferentially to Mice and Human Haematopoietic Progenitor Cells In Vitro

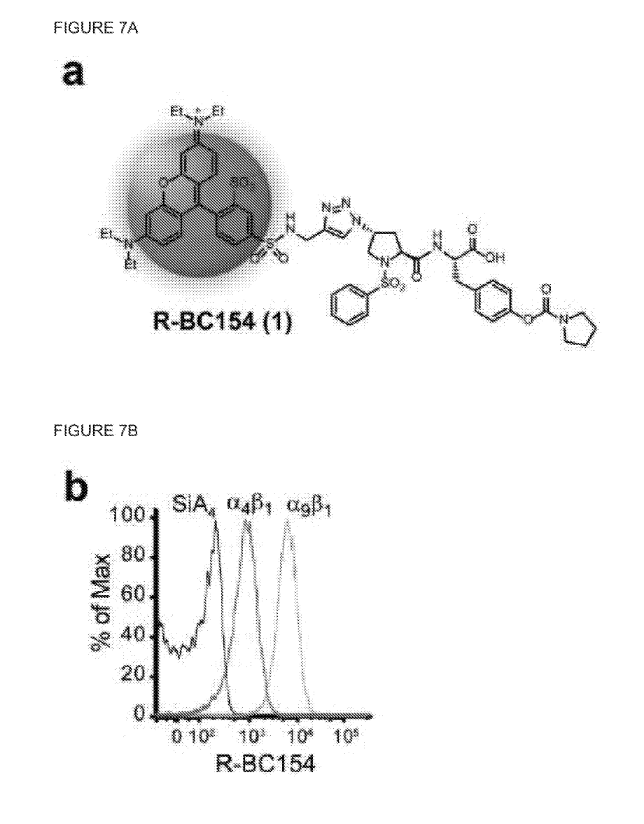

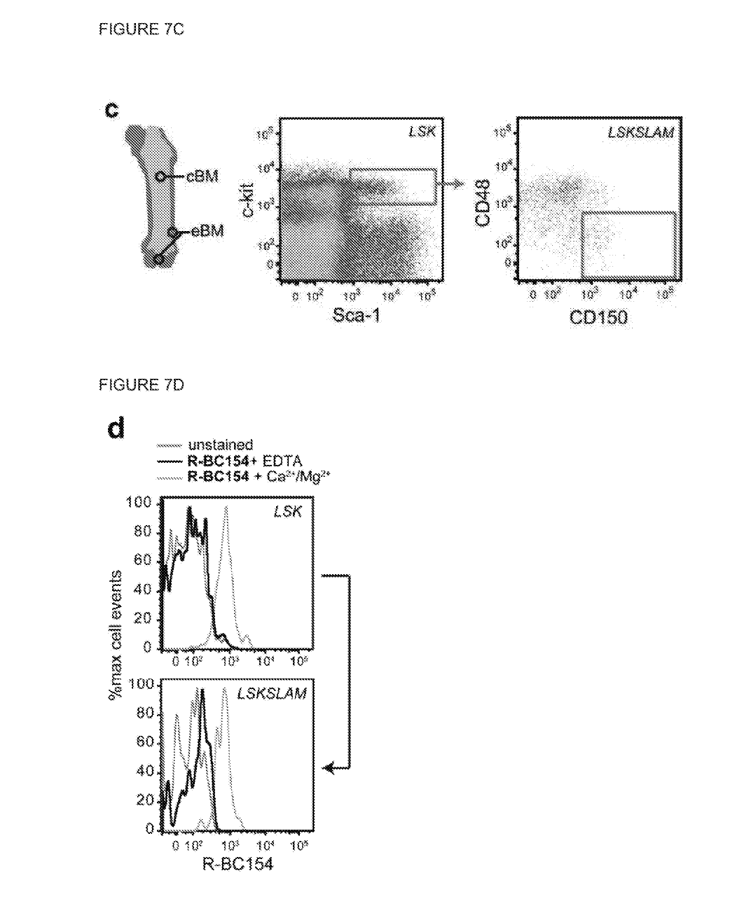

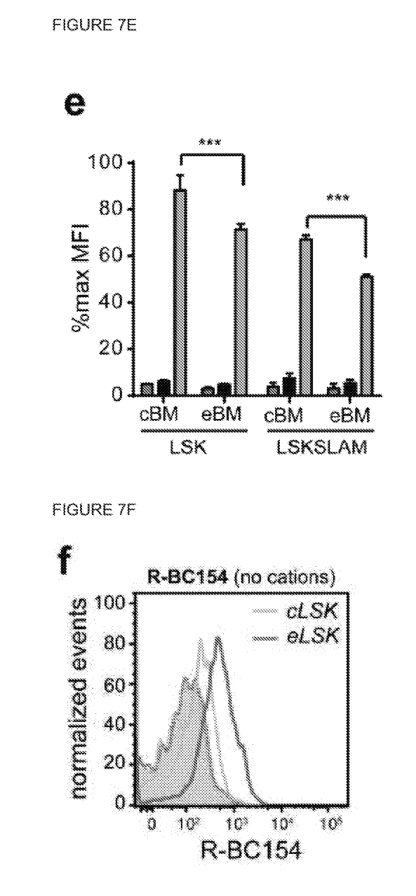

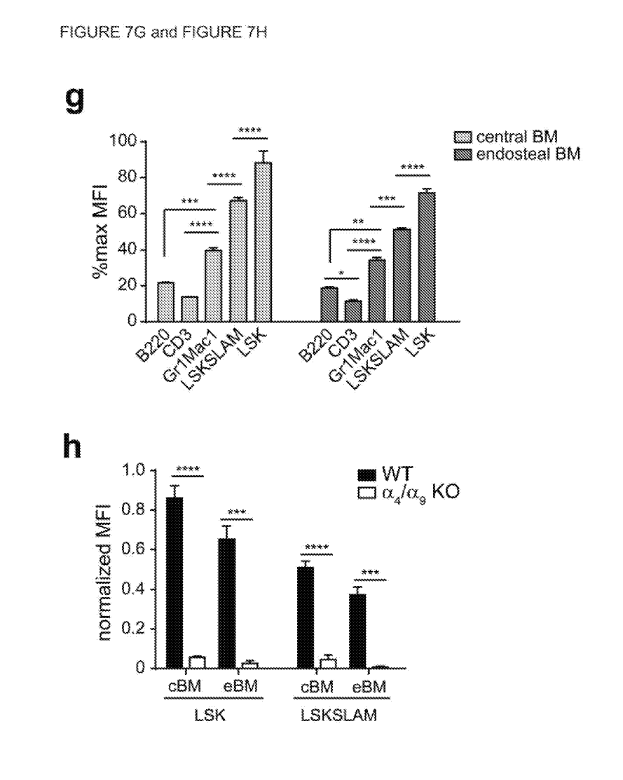

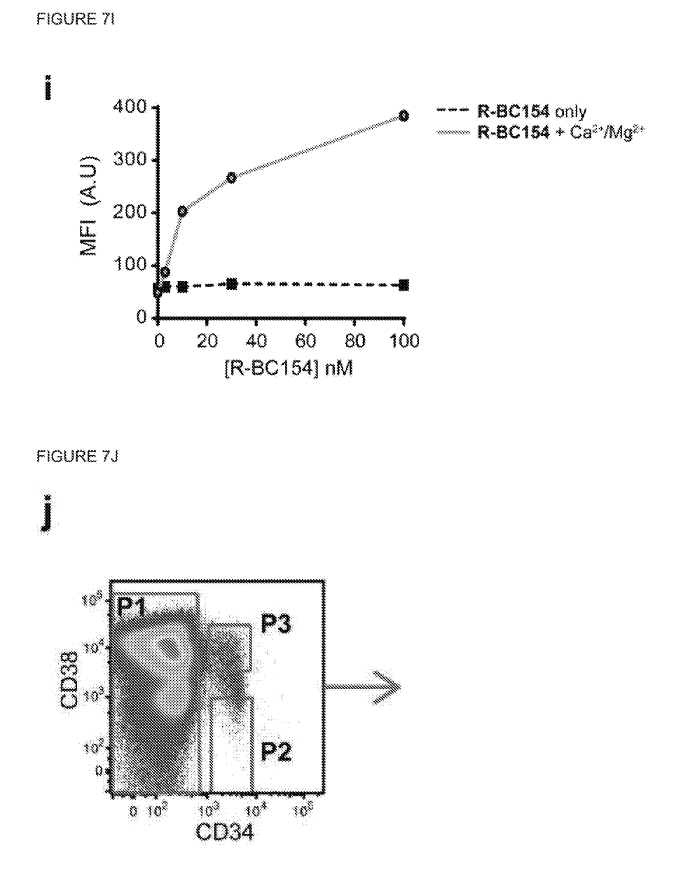

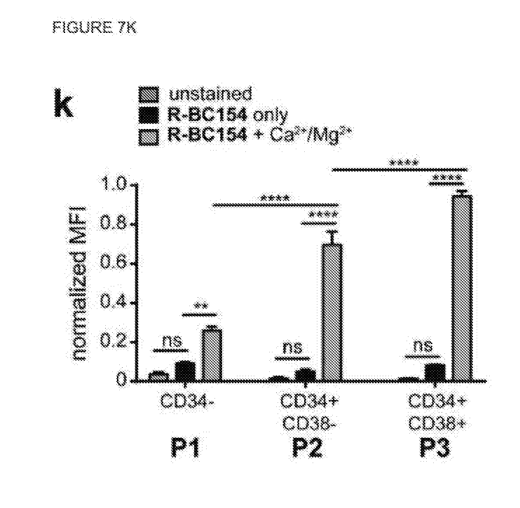

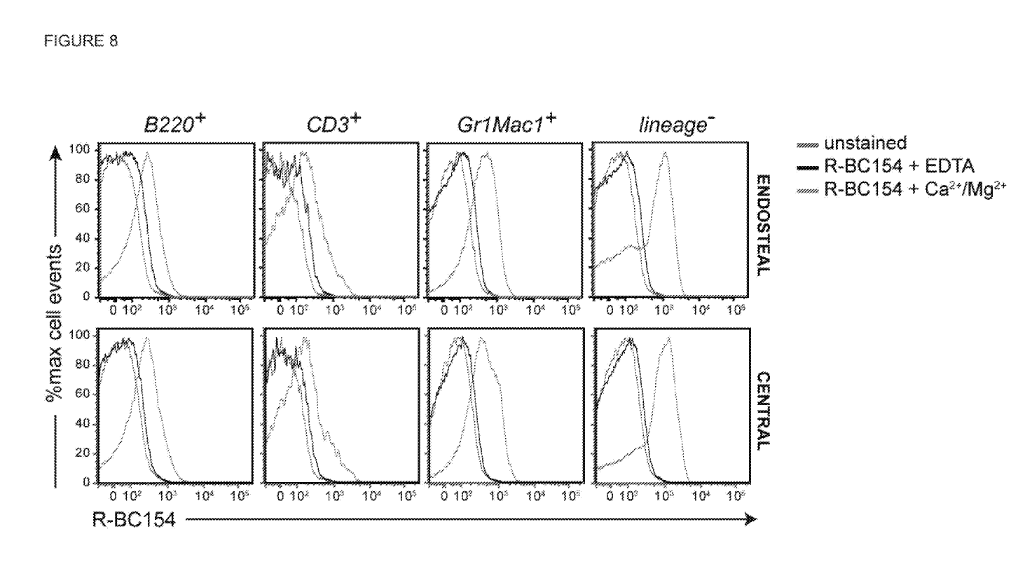

It has been shown in the examples above that R-BC154 (FIG. 7a) binds human glioblastoma LN18 cells overexpressing human α₉β₁ and α₄β₁ integrins (FIG. 7b) only in the presence of divalent metal cations such as Ca²⁺, Mg²⁺ or Mn²⁺, which act to induce conformational changes required for high affinity integrin binding in vitro. To determine whether integrin activation is required for binding to both central and endosteal BM progenitors (Lin⁻Sca-1⁺ckit⁺ cells; LSK) and HSC (LSKCD150⁺CD48⁻ cells; LSKSLAM) (FIG. 7c), R-BC154 binding was assessed in the presence of 1 mM Ca²⁺/Mg²⁺ (FIG. 7d). Under these conditions, greater binding to central LSK and LSKSLAM was observed relative to their endosteal counterparts (p<0.005) (FIG. 7e). Deactivation of surface integrins by co-treatment with EDTA completely abolished activity demonstrating the requirement of integrin activation for efficient R-BC154 binding to HSC and progenitors (FIG. 7e). In the absence of both activating cations and EDTA, R-BC154 binding to endosteal LSK cells was still evident but not to central LSK (FIG. 7f). These results suggest integrins expressed by HSC and progenitors isolated from the endosteal BM remain activated upon harvest.

Since integrin α₄β₁ is ubiquitously expressed on all leukocytes and α₉β₁ is known to be widely expressed on neutrophils, R-BC154 binding to lineage-committed haematopoietic cells was assessed. It was found that activation dependent binding was observed on all lineage committed lymphoid (B220⁺ and CD3+) and myeloid (Gr1/Mac1⁺) progeny isolated from both the central and endosteal BM regions under exogenous activation (FIG. 8). However, this binding was significantly lower relative to LSKSLAM (p<0.0001) and LSK (p<0.0001) cells (FIG. 7g). To confirm whether binding to HSC and progenitor cells is α₄β₁ and α₉β₁ integrin dependent, BM cells devoid of α₄ and α₉ integrins in haematopoietic cells (α₄^{flox/flox}α₉^{flox/flox} vav-cre mice) were treated with R-BC154. Binding was essentially absent on LSK (p<0.005) and LSKSLAM (p<0.005) α₄^{-/-}/α₉^{-/-} cells confirming the requirement of these two integrins for R-BC154 activity (FIG. 7h).

Divalent cation and dose dependent binding of R-BC154 was also confirmed on human cord blood mononuclear cells (MNC) (FIG. 7i). Under activating conditions, greater binding was observed on stem cell enriched CD34⁺CD38⁻ cells compared to lineage-committed CD34⁻ cells, albeit to a lesser extent relative to CD34⁺CD38⁺ progenitor cells (FIGS. 7j and 7k). These results show R-BC154 binding to murine and human haematopoietic cells is divalent metal cation dependent and is also biased towards haematopoietic progenitor cells relative to HSC under exogenous activation in vitro.

### Example 5—R-BC154 Targets HSC and Progenitors Via Intrinsically Activated α₄/α₉ Integrins within the Endosteal Niche In Situ

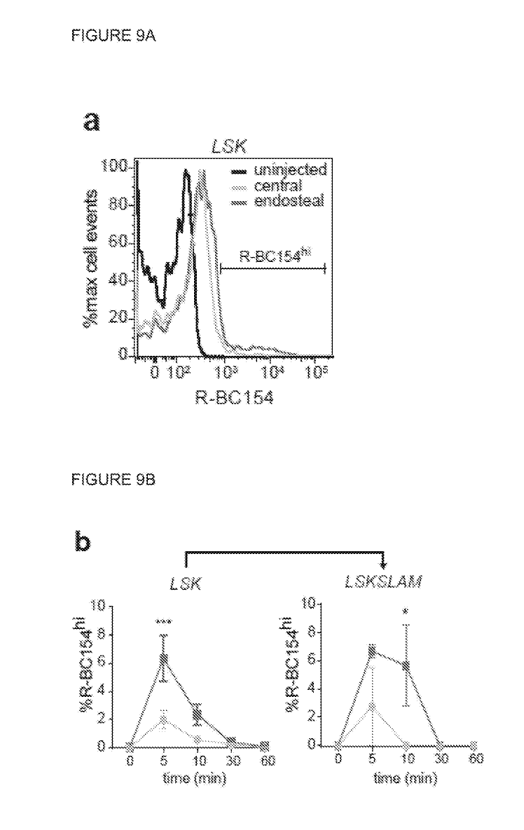

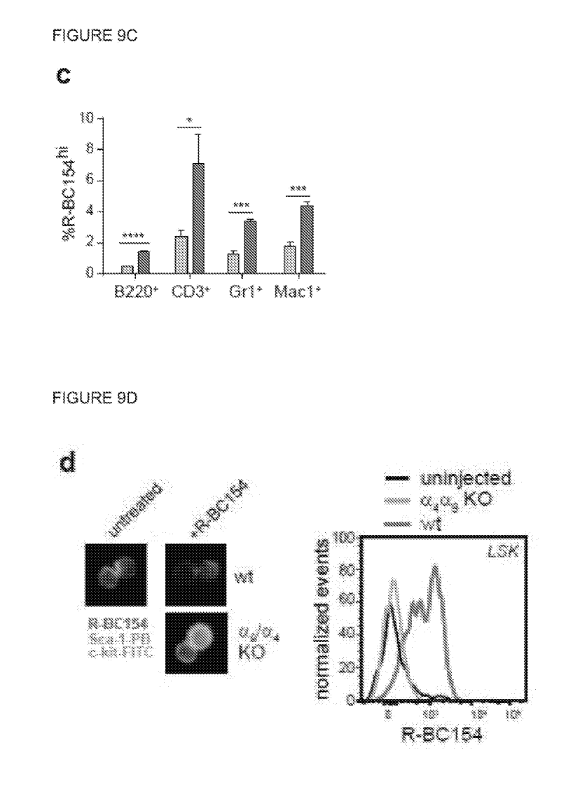

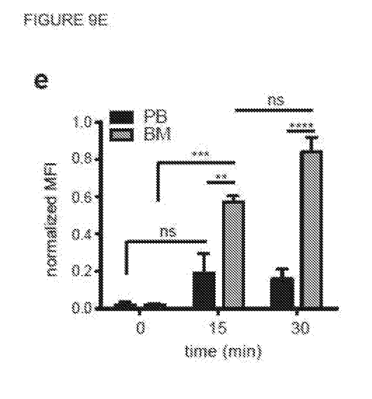

Integrins exist in multiple activation states and their regulation by the stem cell niche is complex and cannot be accurately mimicked or recapitulated in vitro. To assess whether α₉β₁/α₄β₁ integrins expressed by HSC and progenitors within BM are intrinsically and differentially activated in situ, C57Bl/6 mice were injected with R-BC154 prior to immunolabelling for LSKSLAM. LSK cells from both central and endosteal BM regions were effectively labelled with R-BC154 following i.v. administration (FIG. 9a). However, both LSK and LSKSLAM cells within the endosteal BM exhibited a greater proportion of R-BC154^{hi} cells in comparison to their central BM counterparts (FIG. 9b) and is consistent with in vitro experiments performed in the absence of activating divalent metal cations (see FIG. 7f). Lymphoid (B220⁺ and CD3⁺) and myeloid (Gr1⁺ and Mac1⁺) progenies also exhibited a greater proportion of R-BC154^{hi} cells within endosteal BM, suggesting enhanced integrin activation is not restricted to primitive haematopoietic populations (FIG. 9c). No binding of R-BC154 was evident on α₄^{-/-}/α₉^{-/-} LSK cells, confirming the requirement of α₄ and α₉ integrins for in vivo activity (FIG. 9d). These data suggests α₉β₁/α₄β₁ integrins are not only required but are also intrinsically and differentially activated on cells in the endosteal BM region in situ.

### Example 6—Small Molecule α₉β₁/α₄β₁ Integrin Antagonists Rapidly Mobilise HSC and Progenitor Cells

Several murine assays exist for assessing novel mobilization agents in their ability to induce the egress of HSC into PB such as described in Herbert, K. E., et al Biol Blood Marrow Tr 14, 603-621, (2008). Although LSK and LSKSLAM cells in normal PB only constitute −0.005% and −0.0005% of circulating WBC, respectively, sorted LSK and LSKSLAM from PB have been shown to give rise to colony forming cells (CFCs) and thus comprise cells capable of haematopoietic reconstitution capacity. Thus, the determination of LSK and LSKSLAM content in PB was initially used as a surrogate measure of stem and progenitor cell content.

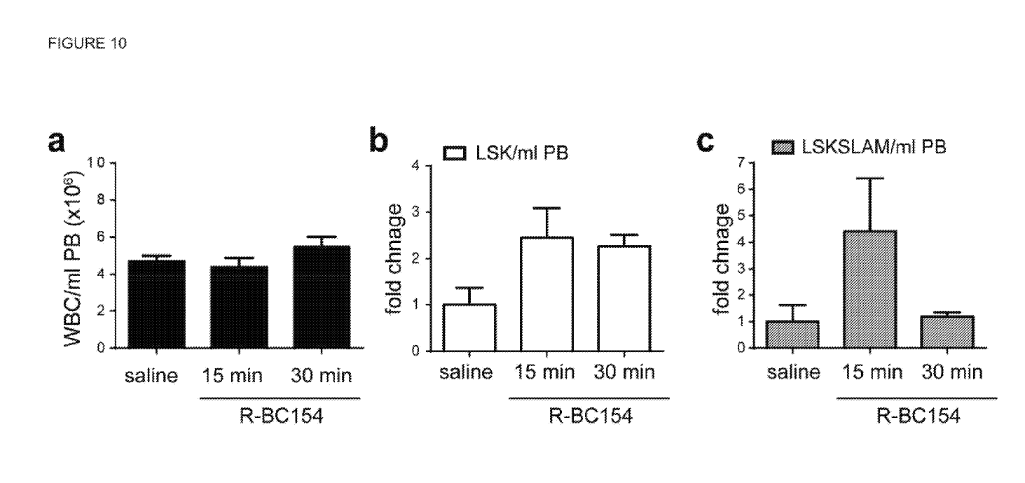

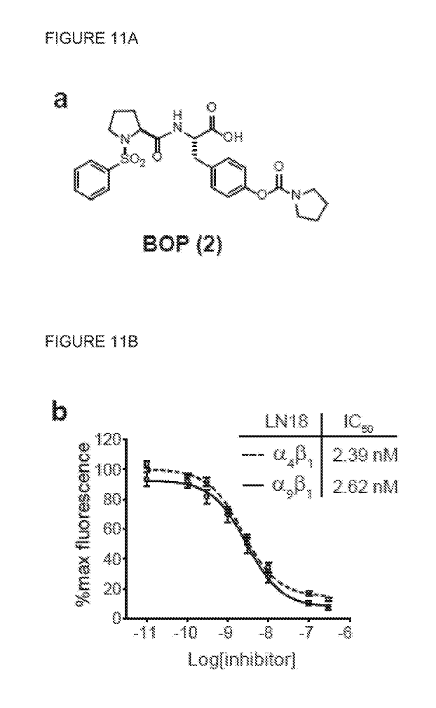

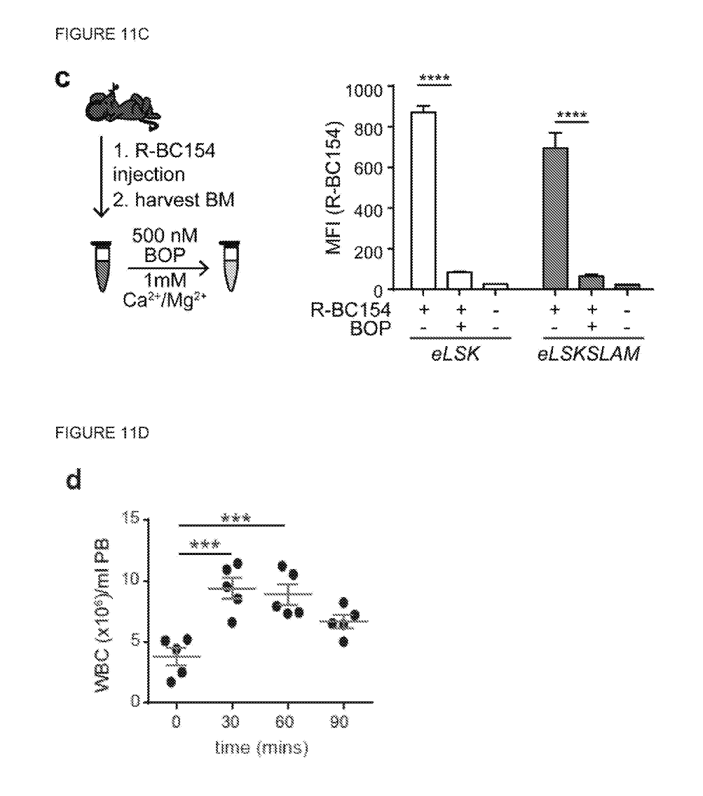

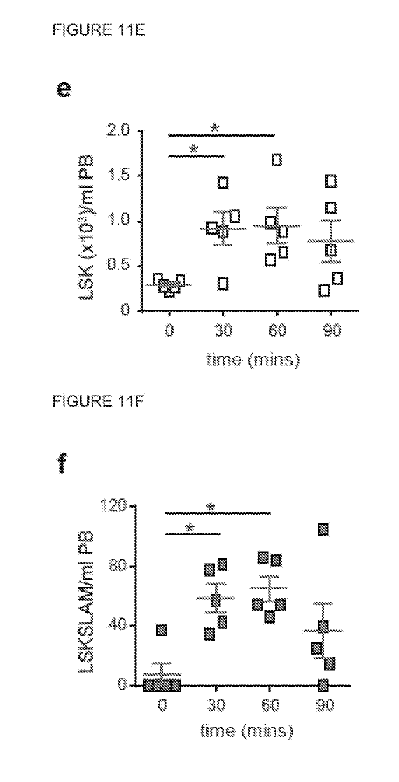

Initially, the rapid clearance of R-BC154 following i.v. administration (<5 minutes) prompted the assessment of other modes of administration and whether s.c. injections would afford sustained binding activity in vivo and thus allow greatest mobilization efficiencies. In contrast to i.v. injections, persistent binding to BM LSK was observed 30 mins post-s.c. treatment (FIG. 9e). Minimal binding was observed on LSK in the PB compared to their BM counterparts, providing further evidence of the requirement of the stem cell niche for effective activation and integrin dependent binding (FIG. 9e). Nevertheless, mice treated with R-BC154 were not found to give significant increases in the number of WBC, LSK or LSKSLAM in PB (FIG. 10). HSC mobilization with α₉β₁/α₄β₁ integrin inhibitors was further investigated using the non-fluorescently labelled BOP (2) (FIG. 11a). BOP was shown to be a potent inhibitor of α₉β₁ and α₄β₁ integrins based on competitive inhibition assays using R-BC154 and overexpressing LN18 cell lines (FIG. 11b) and can inhibit integrin dependent adhesion to VCAM-1 and thrombin-cleaved Opn. Additionally, BOP effectively inhibited α₉β₁ and α₄β₁ integrin binding on HSC and progenitors, demonstrated by competitive displacement of R-BC154 binding to LSK and LSKSLAM under activating conditions (FIG. 11c). Administration of BOP (10 mg/kg) into C57BL/6 mice for up to 90 mins gave significant increases in PB WBC (FIG. 11d), LSK (FIG. 11e) and LSKSLAM (FIG. 11f) compared to the saline control. Unlike R-BC154, greater and more sustained mobilization was observed with BOP, presumably due to its higher binding affinity and slower dissociation-rates.

HSC are known to express several integrin subtypes including αᵥβ₃, α_{L}β₂, α₂β₁, α₅β₁, α₆β₁ α₄β₁ and α₉β₁, many of which have been implicated in HSC retention within BM. In the above examples, evidence is provided to show that inhibition of α₉β₁/α₄β₁ integrins using a small molecule antagonist BOP induces the rapid mobilization of long-term repopulating HSC through inhibition of integrin-dependent binding to VCAM-1 and Opn.

Previous studies have confirmed that inhibition of integrin α₄β₁ and VCAM-1 interactions using neutralizing antibodies or small molecule inhibitors mobilize HSC in both mice and primates, yet the specific cell types that are targeted for mobilization and the location of these target cells within BM has yet to be explored. Using a fluorescent small molecule integrin antagonist (R-BC154) that binds to α₄β₁ and α₉β₁ integrins only when activated by divalent metal cations, it is shown for the first time that the activation state of these two 1 integrins on murine and human HSC are intrinsically activated and differentially specified by the endosteal niche in vivo.

The functional characteristics of the endosteal BM has been thoroughly investigated since the concept of a stem cell niche was originally postulated by Schofield in 1978. Such studies have highlighted the endosteal niche as a hypoxic environment where cell components such as osteoblasts and their associated extracellular matrix proteins are important niche specific regulators of HSC maintenance and function. These examples show that BM cells within the endosteal niche including HSC and progenitor cells express α₉β₁/α₄β₁ integrins that are in a higher affinity binding state beyond what is observed within the central medullary compartment. Although the physiological relevance of this differential integrin activity remains unknown, these observations are consistent with previous reports that HSC-dependent binding between thrombin-cleaved Opn (trOpn) and α₄β₁ and α₉β₁ integrins is restricted to the endosteal bone marrow.

The enhanced activation of integrins by the endosteum was not specific to primitive HSC and progenitors and is unlikely to be restricted to just α₉β₁ and α₄β₁ integrins. Without being limited by theory, one possible explanation for the enhanced integrin activation observed within endosteal BM is its close proximity to bone. Bone, being distinguishable from other microenvironment cells based on its high mineral content, is the primary storage site of inorganic salts of calcium and magnesium as well as trace metals such as manganese, all of which are known to induce α₉β₁ and α₄β₁ integrins to adopt higher affinity ligand-binding conformations. Thus, it remains plausible that high ionic gradients of Ca²⁺ (and perhaps Mg²⁺ and Mn²⁺) emanating from the endosteal surface is responsible for the enhanced integrin binding activity observed. Indeed, this concept has been previously invoked to rationalize the preferential localization of HSC within endosteal BM via recognition of extracellular Ca²⁺ through the G protein-coupled calcium-sensing receptor (CaR). Collectively, these observations further define the unique nature of the endosteal niche, the differential influence it confers in seemingly phenotypically identical cells and provides validation of therapeutic targeting of the stem cell niche for stem cell therapies.

In summary, it is demonstrated that BOP, a small molecule inhibitor of α₄β₁ and α₉β₁ integrins, effectively and rapidly mobilized HSC with long-term multi-lineage engraftment potential.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as broadly described herein.

### REFERENCES

1. J. Grassinger, D. N. Haylock, M. J. Storan, G. O. Haines, B. Williams, G. A. Whitty, A. R. Vinson, C. L. Be, S. H. Li, E. S. Sorensen, P. P. L. Tam, D. T. Denhardt, D. Sheppard, P. F. Choong and S. K. Nilsson, Blood, 2009, 114, 49-59.
2. D. N. Haylock, B. Williams, H. M. Johnston, M. C. P. Liu, K. E. Rutherford, G. A. Whitty, P. J. Simmons, I. Bertoncello and S. K. Nilsson, Stem Cells, 2007, 25, 1062-1069.
3. J. Grassinger, B. Williams, G. H. Olsen, D. N. Haylock and S. K. Nilsson, Cytokine, 2012, 58, 218-225.
4. Nilsson, S. K. et al. Osteopontin, a key component of the haematopoietic stem cell niche and regulator of primitive haematopoietic progenitor cells. Blood 106, 1232-1239, doi:10.1182/blood-2004-11-4422 (2005).
5. Grassinger, J. et al. Thrombin-cleaved osteopontin regulates haematopoietic stem and progenitor cell functions through interactions with alpha(9)beta(1) and alpha(4)beta(1) integrins. Blood 114, 49-59, doi:DOI 10.1182/blood-2009-01-197988 (2009).
6. Bartelmez, S. H. et al. Interleukin-1 Plus Interleukin-3 Plus Colony-Stimulating Factor-I Are Essential for Clonal Proliferation of Primitive Myeloid Bone-Marrow Cells. Experimental Hematology 17, 240-245 (1989).
7. Herbert, K. E., Levesque, J. P., Haylock, D. N. & Princes, M. The use of experimental murine models to assess novel agents of haematopoietic stem and progenitor cell mobilization. Biol Blood Marrow Tr 14, 603-621, doi:DOI 10.1016/j.bbmt.2008.02.003 (2008).
8. Cao, B. et al. Design, synthesis and binding properties of a fluorescent alpha(9)beta(1)/alpha(4)beta(1) integrin antagonist and its application as an in vivo probe for bone marrow haemopoietic stem cells. Org. Biomol. Chem. 12, 965-978, doi:Doi 10.1039/C3ob42332h (2014).
9. Pepinsky, R. B. et al. Comparative assessment of the ligand and metal ion binding properties of integrins alpha 9 beta 1 and alpha 4 beta 1. Biochemistry 41, 7125-7141, doi:Doi 10.1021/Bi020024d (2002).
10. Schofield, R. Relationship between Spleen Colony-Forming Cell and Haematopoietic Stem-Cell—Hypothesis. Blood Cells 4, 7-25 (1978)

The claims defining the invention are as follows:

1. A method for enhancing dislodgement of HSC and their precursors and progenitors thereof from a BM stem cell binding ligand in vivo or ex vivo, said method comprising administering in vivo or ex vivo an effective amount of an antagonist of an α₉ integrin or an active portion thereof to the BM stem cell niche.

2. A method according to claim 1 wherein said method further enhances release of HSC and their precursors and progenitors thereof from the BM stem cell niche.

3. A method according to claim 1 or 2 wherein the method further enhances mobilization of the HSC from the BM stem cell niche.

4. A method according to any one of claims 1 to 4 wherein the α₉ integrin is an α₉β₁ integrin or an active portion thereof.

5. A method according to any one of claims 1 to 4 further including administering an antagonist of α₄ integrin or an active portion thereof.

6. A method according to claim 5 wherein the α₄ integrin is an antagonist of α₄β₁ or an active portion thereof.

7. A method according to any one of claims 1 to 6 wherein the antagonist cross-reacts with α₉ and α₄, and optionally cross-reacts with α₉β₁ and α₄β₁.

8. A method according to any one of claims 1 to 7 wherein the antagonist is a compound of Formula (I) or a pharmaceutically acceptable salt thereof having the formula: 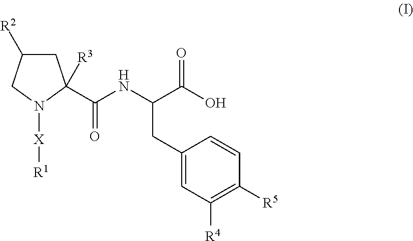
wherein
X is selected from the group consisting of a bond and —SO₂—;
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

9. A method according to claim 8 wherein:
R⁴ is H; and
R⁵ is —OR⁷.

10. A method according to claim 8 or claim 9 wherein:
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of C₁-C₄ alkyl, —CN, —O(C₁-C₄ alkyl) and optionally substituted heteroaryl;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl, optionally substituted aryl or
R¹⁴ and R¹⁵, together with the nitrogen to which they are attached, form an optionally substituted heterocycloalkyl ring; and
n is 1 or 2.

11. A method according to any one of claims 8 to 10 wherein:
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of C₁-C₄ alkyl, —CN, —O(C₁-C₄ alkyl) and 5-tetrazolyl;
R¹³ is 2-pyrrolyl;
R¹⁴ and R¹⁵ are each independently C₁-C₄ alkyl or
R¹⁴ and R¹⁵, together with the nitrogen to which they are attached, form an optionally substituted pyrrolidinyl or morpholinyl ring; and
n is 1 or 2.

12. A method according to any one of claims 8 to 11 wherein the compound of Formula (I) is: 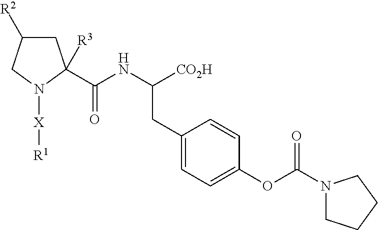
or a pharmaceutically acceptable salt thereof.

13. The method according to any one of claims 8 to 12 wherein R¹ is optionally substituted phenyl.

14. The method according to claim 13, wherein the phenyl is optionally substituted with at least one halogen group.

15. The method according to any one of claims 8 to 14 wherein the compound of Formula (I) is 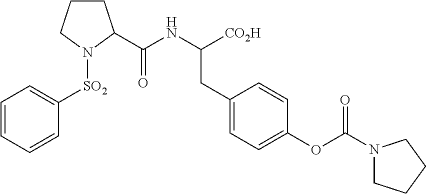 [SMILES: O=C(O)C(Cc1ccc(OC(=O)N2CCCC2)cc1)NC(=O)C1CCCN1S(=O)(=O)c1ccccc1]
or a pharmaceutically acceptable salt thereof.

16. The method according to any one of claims 8 to 15, wherein the compound of formula (I) is 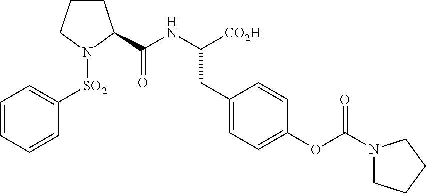 [SMILES: O=C(O)[C@H](Cc1ccc(OC(=O)N2CCCC2)cc1)NC(=O)[C@@H]1CCCN1S(=O)(=O)c1ccccc1]
or a pharmaceutically acceptable salt thereof.

17. A method according to any one of claims 1 to 16 wherein the antagonist is administered in the absence of G-CSF.

18. A method according to any one of claims 1 to 17 wherein the α₉ integrin antagonist is administered intravenously, intradermally, subcutaneously, intramuscularly, transdermally, transmucosally or intraperitoneally; optionally the antagonist is administered intravenously or subcutaneously.

19. A method according to any one of claims 5 to 18 wherein the α₉ integrin antagonist is administered simultaneously, consecutively or in combination with an α₄ integrin antagonist.

20. A method according to any one of claims 1 to 19 wherein the HSC are derived from bone marrow.

21. A method according to claim 20 wherein the HSC are derived from the stem cell niche, optionally the central or endosteal niche of the bone marrow.

22. A method according to anyone of claims 1 to 21 wherein the HSC are endosteal progenitor cells selected from the group including CD34⁺ cells, CD38⁺ cells, CD90⁺ cells, CD133⁺ cells, CD34⁺CD38⁻ cells, lineage-committed CD34⁻ cells, or CD34⁺CD38⁺ cells.

23. A composition for enhancing dislodgement of HSC from a BM stem cell binding ligand said composition comprising an antagonist of α₉ integrin or an active portion thereof.

24. A composition according to claim 23 further enhancing release of HSC from a BM stem cell binding ligand.

25. A composition according to claim 24 further enhancing mobilization of HSC from a BM stem cell niche to PB.

26. A composition according to any one of claims 23 to 25 wherein the α₉ integrin is an α₉β₁ integrin or an active portion thereof.

27. A composition according to any one of claims 23 or 26 further including an antagonist of α₄ integrin or an active portion thereof.

28. A composition according to claim 27 wherein the α₄ integrin is an antagonist of α₄β₁ or an active portion thereof.

29. A composition according to any one of claims 23 to 28 wherein the antagonist cross-reacts with α₉ and α₄, and optionally cross-reacts with α₉β₁ and α₄β₁.

30. A composition according to any one of claims 23 to 29 wherein the α₉ integrin antagonist is a compound of Formula (I) or a pharmaceutically acceptable salt thereof having the formula: 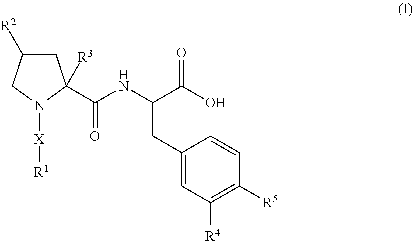
wherein
X is selected from the group consisting of a bond and —SO₂—;
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

31. A composition according to claim 30 wherein:
R⁴ is H; and
R⁵ is —OR⁷.

32. A composition according to claim 23 or 31 wherein:
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of —CN, —O(C₁-C₄ alkyl) and optionally substituted heteroaryl;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl, optionally substituted aryl or
R¹⁴ and R¹⁵, together with the nitrogen to which they are attached, form an optionally substituted heterocycloalkyl ring; and
n is 1 or 2.

33. A composition according to any one of claims 23 to 32 wherein:
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of C₁-C₄ alkyl, —CN, —O(C₁-C₄ alkyl) and 5-tetrazolyl;
R¹³ is 2-pyrrolyl;
R¹⁴ and R¹⁵ are each independently C₁-C₄ alkyl or
R¹⁴ and R¹⁵, together with the nitrogen to which they are attached, form an optionally substituted pyrrolidinyl or morpholinyl ring; and
n is 1 or 2.

34. A composition according any one of claims 23 to 33 wherein the compound of Formula (I) is: 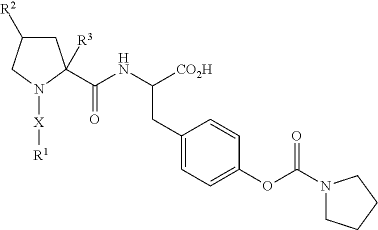
or a pharmaceutically acceptable salt thereof.

35. A composition according to any one of claims 23 to 34 wherein R¹ is optionally substituted phenyl.

36. The composition according to claim 35, wherein the phenyl is optionally substituted with at least one halogen group.

37. The composition according to any one of claims 23 to 36 wherein the compound of Formula (I) is 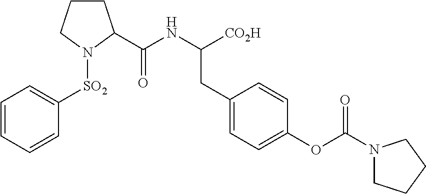 [SMILES: O=C(O)C(Cc1ccc(OC(=O)N2CCCC2)cc1)NC(=O)C1CCCN1S(=O)(=O)c1ccccc1]
or a pharmaceutically acceptable salt thereof.

38. The composition according to any one of claims 23 to 37, wherein the compound of formula (I) is 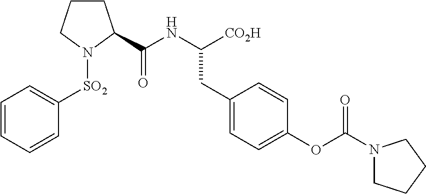 [SMILES: O=C(O)[C@H](Cc1ccc(OC(=O)N2CCCC2)cc1)NC(=O)[C@@H]1CCCN1S(=O)(=O)c1ccccc1]
or a pharmaceutically acceptable salt thereof.

39. A composition according to any one of claims 30 to 38 for administering in the absence of G-CSF.

40. A composition according to any one of claims 30 to 39 for administering intravenously, intradermally, subcutaneously, intramuscularly, transdermally, transmucosally or intraperitoneally; optionally the composition is administered intravenously or subcutaneously.

41. A composition according to any one of claims 30 to 40 wherein the α₉ integrin antagonist is administered simultaneously, consecutively or in combination with an α₄ integrin antagonist.

42. A composition according to any one of claims 30 to 41 wherein the HSC are derived from bone marrow.

43. A composition according to any one of claims 30 to 42 wherein the HSC are derived from the stem cell niche, optionally the central or endosteal niche of the bone marrow.

44. A composition according to anyone of claims 30 to 43 wherein the HSC are endosteal progenitor cells selected from the group including CD34⁺ cells, CD38⁺ cells, CD90⁺ cells, CD133⁺ cells, CD34⁺CD38⁻ cells, lineage-committed CD34⁻ cells, or CD34⁺CD38⁺ cells.

45. A method of harvesting HSC from a subject said method comprising:
administering an effective amount of an antagonist of α₉ integrin or an active portion thereof to a subject wherein said effective amount enhances dislodgement of HSC and their precursors and progenitors thereof from a BM stem cell binding ligand in a BM stem cell niche;
mobilizing the dislodged HSC to PB; and harvesting the HSC from the PB.

46. A method according to claim 45 wherein the α₉ integrin antagonist is administered in the absence of G-CSF.

47. A method according to claim 45 wherein the HSC are further mobilized by the use of other HSC mobilizing agents such as, but not limited to interleukin-17, cyclophosphamide (Cy), Docetaxel and granulocyte-colony stimulating factor (G-CSF).

48. A method according to claim 45 or 46 the effective amount of the integrin antagonist is in the range 25-1000 μg/kg body weight, more preferably 50-500 μg/kg body weight, most preferably 50-250 μg/kg body weight.

49. A cell composition comprising HSC obtained from a method according to any one of claims 45 to 48.

50. A method for the treatment of a haematological disorder said method comprising administering a cell composition according to claim 23 to 44 or a cell composition according to claim 49.

51. A method for the treatment of a haematological disorder in a subject said method comprising administering a therapeutically effective amount of an antagonist of α₉ integrin or an active portion thereof to the subject to enhance dislodgement, release or mobilization of HSC from the BM to the PB.

52. A method according to claim 51 wherein the α₉ integrin is an α₉β₁ integrin or an active portion thereof.

53. A method according to claim 51 or 52 further including administering an antagonist of α₄ integrin or an active portion thereof.

54. A method according to claim 53 wherein the α₄ integrin is an antagonist of α₄β₁ or an active portion thereof.

55. A method according to any one of claims 51 to 54 wherein the antagonist cross-reacts with α₉ and α₄, and optionally cross-reacts with α₉β₁ and α₄β₁.

56. A method according to any one of claims 50 to 55 wherein the antagonist is a compound of Formula (I) or a pharmaceutically acceptable salt thereof having the formula: 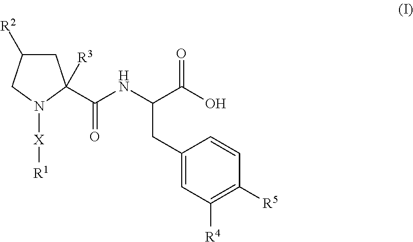
wherein
X is selected from the group consisting of a bond and —SO₂—;
R¹ is selected from the group consisting of H, alkyl, optionally substituted aryl and optionally substituted heteroaryl;
R² is selected from the group consisting of H and a substituent group;
R³ is selected from the group consisting of H and C₁-C₄ alkyl;
R⁴ is selected from the group consisting of H and —OR⁶;
R⁵ is selected from the group consisting of H and —OR⁷;
provided that when R⁴ is H then R⁵ is —OR⁷ and when R⁴ is —OR⁶ then R⁵ is H;
R⁶ is selected from the group consisting of H, C₁-C₄ alkyl, —(CH₂)ₙ—R⁸, —C(O)R⁹ and —C(O)NR¹⁰R¹¹;
R⁷ is selected from the group consisting of H, C₁-C₄ alkyl, —(CH₂)ₙ—R¹², —C(O)R¹³ and —C(O)NR¹⁴R¹⁵;
R⁸ is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R⁹ is selected from the group consisting of optionally substituted cycloalkyl, optionally substituted heterocycloalkyl, optionally substituted aryl and optionally substituted heteroaryl;
R¹⁰ and R¹¹, together with the nitrogen to which they are attached, form an optionally substituted heterocycloalkyl ring;
R¹² is selected from the group consisting of optionally substituted alkyl, optionally substituted aryl, optionally substituted heteroaryl, —O(C₁-C₄ alkyl), —C(O)—(C₁-C₄ alkyl), —C(O)O—(C₁-C₄ alkyl) and —CN;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl and optionally substituted aryl, or
R¹⁴ and R¹⁵, together with the nitrogen to which they are attached, form an optionally substituted heterocycloalkyl ring; and
n at each occurrence is an integer in the range of from 1 to 3.

57. A method according to claim 56 wherein:
R⁴ is H; and
R⁵ is —OR⁷.

58. A method according to claim 56 or claim 57 wherein:
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of —CN, —O(C₁-C₄ alkyl) and optionally substituted heteroaryl;
R¹³ is selected from the group consisting of optionally substituted cycloalkyl, optionally substituted aryl and optionally substituted heteroaryl;
R¹⁴ and R¹⁵ are each independently selected from the group consisting of C₁-C₄ alkyl, optionally substituted aryl or
R¹⁴ and R¹⁵, together with the nitrogen to which they are attached, form an optionally substituted heterocycloalkyl ring; and
n is 1 or 2.

59. A method according to any one of claims 56 to 58 wherein:
R⁷ is selected from the group consisting of C₁-C₄ alkyl, —(CH₂)ₙ—R¹², —C(O)R¹³ and —C(O)NR¹⁴R¹⁵;
R¹² is selected from the group consisting of C₁-C₄ alkyl, —CN, —O(C₁-C₄ alkyl) and 5-tetrazolyl;
R¹³ is 2-pyrrolyl;
R¹⁴ and R¹⁵ are each independently C₁-C₄ alkyl or
R¹⁴ and R¹⁵, together with the nitrogen to which they are attached, form an optionally substituted pyrrolidinyl or morpholinyl ring; and
n is 1 or 2.

60. A method according any one of claims 56 to 59 wherein the compound of Formula (I) is: 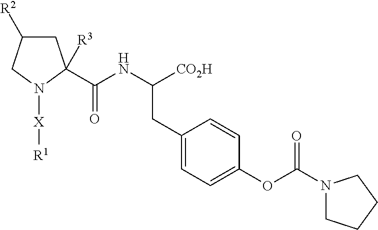
or a pharmaceutically acceptable salt thereof.

61. The method according to any one of claims 56 to 60 wherein R¹ is optionally substituted phenyl.

62. The method according to claim 61, wherein the phenyl is optionally substituted with at least one halogen group.

63. The method according to any one of claims 56 to 62 wherein the compound of Formula (I) is 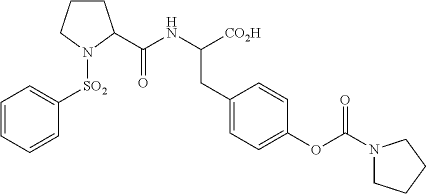 [SMILES: O=C(O)C(Cc1ccc(OC(=O)N2CCCC2)cc1)NC(=O)C1CCCN1S(=O)(=O)c1ccccc1]
or a pharmaceutically acceptable salt thereof.

64. The method according to any one of claims 56 to 63, wherein the compound of formula (I) is 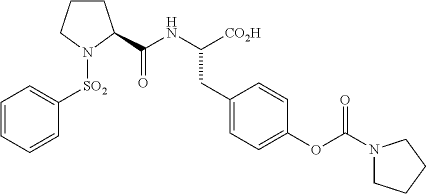 [SMILES: O=C(O)[C@H](Cc1ccc(OC(=O)N2CCCC2)cc1)NC(=O)[C@@H]1CCCN1S(=O)(=O)c1ccccc1]
or a pharmaceutically acceptable salt thereof.

65. A method according to any one of claims 51 to 64 wherein the antagonist is administered in the absence of G-CSF.

66. A method according to any one of claims 51 to 65 wherein the α₉ integrin antagonist is administered intravenously, intradermally, subcutaneously, intramuscularly, transdermally, or transmucosally; optionally the antagonist is administered intravenously or subcutaneously.

67. A method according to any one of claims 51 to 66 wherein the α₉ integrin antagonist is administered simultaneously, consecutively or in combination with an α₄ integrin antagonist.

68. A method according to any one of claims 51 to 67 wherein the haematological disorder is selected from the group including immunosuppression, chronic illness, traumatic injury, degenerative disease, infection, or combinations thereof; a disease or condition of the skin, digestive system, nervous system, lymph system, cardiovascular system, endocrine system, or combinations thereof; osteoporosis, Alzheimer's disease, cardiac infarction, Parkinson's disease, traumatic brain injury, multiple sclerosis, cirrhosis of the liver, or combinations thereof; neuroblastoma, myelodysplasia, myelofibrosis, breast cancer, renal cell carcinoma, or multiple myeloma; haematopoietic neoplastic disorder; autoimmune disease; or non-malignant disorder.

69. A method according to claim 68 wherein the haematological disorder is acute lymphoblastic leukemia (ALL) selected form the group including B-lineage ALL and T-lineage ALL, chronic lymphocytic leukemia (CLL), prolymphocytic leukemia (PLL), hairy cell leukemia (HLL) and Waldenstrom's macroglobulinemia (WM).

70. A method of transplanting HSC into a patient, said method comprising
administering an α₉ integrin antagonist to a subject to dislodge HSC from a BM stem cell binding ligand;
releasing and mobilizing the HSC from the BM to the PB;
harvesting HSC from the PB from the subject; and
transplanting the HSC to the patient.

71. A method according to claim 70 wherein the HSC are endosteal progenitor cells and are selected from the group comprising CD34⁺, CD38⁺, CD90⁺, CD133⁺, CD34⁺CD38⁻ cells, lineage-committed CD34⁻ cells, or CD34⁺CD38⁺ cells
